(19) United States
(12) Patent Application Publication
(10) Pub. No.: US 2026/0358337 A1
(43) Pub. Date: Nov. 12, 2026

(54) COMPOSITIONS AND METHODS OF ANALYZING A SAMPLE FOR ANTIGEN-SPECIFIC IMMUNE CELLS

(71) Applicant: Immunoracle Inc., Natick, MA (US)

(72) Inventors: Yujiang Geno SHI, Boston, MA (US); Gang SONG, Natick, MA (US)

(21) Appl. No.: 19/481,061

(22) PCT Filed: May 3, 2024
(86) PCT No.: PCT/CN2024/091080

§ 371 Date: Nov. 3, 2025

Related U.S. Application Data
(60) Provisional application No. 63/463,886, filed on May 3, 2023.

(51) Int. Cl. G01N 33/569; G01N 33/543

(52) U.S. Cl. CPC G01N 33/56972; G01N 33/54313; G01N 33/56977; G01N 2333/70539

(57) ABSTRACT

Methods of analyzing a sample from one or more individuals for the presence or absence of immune cells capable of binding to an antigen peptide are provided. Methods of detecting or treating a cancer or tumor, pathogen infection or autoimmune diseases are also provided. Display moieties that have a particle associated with a plurality of MHC-peptide complexes in which at least two MHC-peptide complexes are different are provided. Bait compositions that have a plurality of display moieties and methods of analyzing immune cells by using the display moieties or bait compositions as a screening tool are also provided.

### CROSS REFERENCE TO RELATED APPLICATION

This application claims the benefit of U.S. provisional application No. 63/463,886, filed May 3, 2023, the contents of which are incorporated herein by reference in their entirety for all purposes.

### REFERENCE TO AN ELECTRONIC SEQUENCE LISTING

The contents of the electronic sequence listing (230062000440SEQLIST. xml; Size: 101,021 bytes; and Date of Creation: May 1, 2024) is herein incorporated by reference in its entirety.

### FIELD

The application relates to compositions and methods of analyzing a sample for antigen-specific immune cells.

### BACKGROUND

Recognition of peptide-major histocompatibility complex (pMHC or MHC-peptide complex) class I and II molecules by immune cells (e.g., T cells) plays a very important role in the control of intracellular pathogens and cancer cells through efficient immune cell (e.g., T cell) stimulation. The overall low avidity of the TCR to the pMHC complex and the fast off-rate of this interaction make it difficult to detect antigen-specific T cells using the soluble monomeric pMHC complex. To overcome these shortcomings, MHC multimers have been prepared using antibody dimerization or tetravalent binding of biotin-streptavidin, and these MHC dimers or tetramers show increased avidity for their cognate TCR. To date, MHC multimers have been successfully used to visualize antigen-specific CD8 T cells ex vivo by flow cytometry.

Conventional MHC tetramers are constructed by tetramerization of C-terminal biotinylated pMHC monomers with streptavidin molecules. MHC tetramers have been proven to be versatile and useful for detecting αβ T cells through flow cytometry and in situ staining. The well-established characteristics of MHC tetramers and their public and commercial availability have led to their widespread usage in T-cell research. MHC tetramer-staining technology has been expanding to include new research areas such as enrichment of rare T cells, construction of large libraries and high-throughput epitope screening, cytometry by time-of-flight mass spectrometry (CyTOF), and combinatorial staining for multiple detection.

An MHC pentamer consisting of five pMHC complexes arranged in a planar configuration is commercially produced and has increased sensitivity for staining T cells with the low-affinity TCR because of its higher valency than that of dimers or tetramers. More recently, another commercially available pMHC complex linked to a dextran backbone, a pMHC dextramer, has also been found to be useful for the detection of T cells with low-affinity TCRs because of its increased valency. See e.g., Mol Cells. 2021 May 31; 44 (5): 328-334.

A wide variety of assays can be used to characterize immune cell (e.g., T cell) responses in order to determine the state and capability of the immune system. Such studies can reveal fundamental mechanisms underlying immunity aid the design of clinical diagnostics, help develop intervention therapies, and determine signatures of effective immune responses. However, because antigen-specific immune cells are often a very small percentage of the cells that can be obtained from a sample (e.g., a blood sample), effective detection and analysis of such cells could be challenging.

The disclosures of all publications, patents, patent applications and published patent applications referred to herein are hereby incorporated herein by reference in their entirety.

### BRIEF SUMMARY

The present application in one aspect provides methods of analyzing a sample from one or more individuals for the presence or absence of immune cells capable of binding to an antigenic peptide, the method comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization.

The present application in another aspect provides methods of analyzing a sample from one or more individuals for the presence or absence of immune cells capable of binding to an antigenic peptide, the method comprising: a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety.

In some embodiments according to any of the methods described above, the sample is obtained from a single individual.

In some embodiments according to any of the methods described above, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the method further comprises determining the unique barcode associated with the immune cells bound to the display moiety, thereby identifying the sample from which the immune cells are derived.

The present application in another aspect provides methods of analyzing a plurality of samples from one or more individuals for the presence or absence of immune cells capable of binding to an antigenic peptide, comprising: a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived.

In some embodiments according to any of the methods described above, the immune cells comprise T cells, optionally the immune cells comprise no more than about 10%, 5%, 4%, 3%, 2% or 1% T cells specifically binding to antigenic peptide in the bait composition. In some embodiments, the immune cells are selected from the group consisting of: a cytotoxic T cell, a memory T cell, and a tumor infiltrating T cell.

In some embodiments according to any of the methods described above, the immune cells comprise B cells.

In some embodiments according to any of the methods described above, the method further comprises removing unbound display moiety following contacting the immune cells with the agent recognizing the display moiety and prior to detecting the immune cells bound to the display moiety.

In some embodiments according to any of the methods described above, the antigenic peptide is associated with a cancer or tumor antigen, optionally wherein the antigenic peptide is a neoantigen peptide. In some embodiments, the sample is obtained from one or more individuals exhibiting no pathological symptom of a cancer.

In some embodiments according to any of the methods described above, the antigenic peptide is associated with a pathogen antigen, optionally wherein the antigenic peptide is associated with a virus, a bacteria, or a fungus antigen.

In some embodiments according to any of the methods described above, the antigenic is an autoantigenic peptide associated with an autoantigen.

In some embodiments according to any of the methods described above, the immune cells are immobilized on the solid substrate such as a bead. In some embodiments, the bead is associated with an antibody recognizing the immune cells, optionally wherein the antibody is a CD8 antibody.

In some embodiments according to any of the methods described above, the display moiety comprises two or more antigenic peptides, optionally wherein the display moiety comprises four antigenic peptides. In some embodiments, the two or more antigenic peptides in the display moiety are the same.

In some embodiments according to some of the methods described above, the display moiety comprises a library of antigenic peptides for a mutation associated with a cancer or tumor, wherein the library comprises at least about 5, 10, 12, 15, 18 or 20 distinct neoantigenic peptides.

In some embodiments according to any of the methods described above, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues.

In some embodiments according to some of the methods described above, the antigenic peptide is a neoantigenic peptide comprising a mutation relative to a wildtype peptide, optionally at the third amino acid position counting from the N-terminus.

In some embodiments according to any of the methods described above, the antigenic peptide has low immunogenicity.

In some embodiments according to any of the methods described above, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the antigenic peptide is about 8 to about 10 amino acids long. In some embodiments, the MHC comprises a MHC class II molecule. In some embodiments, the MHC class II molecule is selected from the group consisting of HLA-DQ and HLA-DR. In some embodiments, the antigenic peptide is about 10 to about 20 amino acids long. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03.

In some embodiments according to any of the methods described above, the display moiety comprises a particle. In some embodiments, the particle in the display moiety is selected from the group consisting of: a surface, a nanoparticle, a bead, and a polymer. In some embodiments, the particle in the display moiety is a dextran particle. In some embodiments, the particle in the display moiety is a magnetic nanoparticle or polystyrene nanoparticle. In some embodiments, the particle in the display moiety is an agarose bead or a sepharose bead. In some embodiments, the antigenic peptide or MHC is directly attached to the particle in the display moiety. In some embodiments, the antigenic peptide or MHC is attached to the particle in the display moiety via a binding pair comprising a first binding component attached to the antigenic peptide and a second binding component bound to the particle.

In some embodiments according to any of the methods described above, the display moiety comprises a cell. In some embodiments, the cell comprises a polynucleotide encoding the antigenic peptide. In some embodiments, the polynucleotide encodes a plurality of antigenic peptides.

In some embodiments according to any of the methods described above, the display moiety further comprises a detectable label. In some embodiments, the detectable label is a fluorophore.

In some embodiments according to any of the methods described above, the method further comprises isolating immune cells bound to the display moiety or removing the display moiety or immune cells not bound to each other.

In some embodiments according to any of the methods described above, the method further comprises phenotyping immune cells bound to the display moiety.

In some embodiments according to any of the methods described above, the method further comprises sequencing one or more nucleic acids in immune cells bound to the display moiety, optionally wherein the method further comprises analyzing the sequences of the one or more nucleic acids, optionally wherein the one or more nucleic acids is a TCR, CD8, CD45 sequences or any specific sequence that is characteristic of the captured immune cells, optionally wherein analyzing the sequences of the one or more nucleic acids comprises whole genome sequencing and/or RNAseq sequencing, alternatively, by a array of primer sequence-specific q-PCR, and/or RT-PCR.

In some embodiments according to any of the methods described above, the method further comprises subjecting the immune cell bound to the display moiety to mass spectrometry analysis.

In some embodiments according to any of the methods described above, the method further comprises identifying one or more epigenetic modifications in the immune cell, optionally wherein the more or more epigenetic modifications comprises DNA methylation and/or histone glycosylation.

In some embodiments according to any of the methods described above, the individual is a human. In some embodiments, the individual is at least about 50 years old.

In some embodiments according to some of the methods described above, the individual a) has not previously been diagnosed as having a cancer, optionally wherein the individual is at risk of having cancer, or b) the individual has been previously treated for cancer and exhibits no pathological symptom of a cancer after the treatment.

In some embodiments according to some of the methods described above, the individual a) has not been diagnosed as having an infection by a pathogen, optionally wherein the individual is at risk of having the infection, and/or b) exhibits no pathological symptom of an infection by a pathogen, optionally exhibits no pathological symptom of an infection by the pathogen.

In some embodiments according to some of the methods described above, wherein the individual a) has not been diagnosed as having an immune response against the autoantigen, optionally wherein the individual is at risk of developing the immune response against the autoantigen, and/or b) exhibits no pathological symptom of an immune response (e.g., an autoimmune disease or disorder), optionally exhibits no pathological symptom of an immune response against the autoantigen.

In some embodiments according to any of the methods described above, the sample is selected from the group consisting of: blood, plasma, and a peripheral blood mononuclear cell (PBMC) sample.

In some embodiments according to any of the methods described above, the bait composition comprises a plurality of different display moieties. In some embodiments, the each of the plurality of different display moieties in the bait composition comprises a different antigenic peptide. In some embodiments, the plurality of different display moieties in the bait composition comprises at least two different display moieties, each comprising a different MHC molecule. In some embodiments, the plurality of different display moieties in the bait composition comprises at least four different display moieties, each comprising a different MHC molecule. In some embodiments, the plurality of different display moieties in the bait composition comprises at least 100 different display moieties, each comprising a different MHC molecule. In some embodiments, each of the different display moieties comprising different MHC molecules comprises a different detectable label. In some embodiments, the different detectable labels comprise fluorophore(s).

In some embodiments according to any of the methods described above, the agent comprises an antibody that binds to the display moiety.

In another aspect, the present application provides methods of detecting antigen-specific immune cells in an individual, comprising: analyzing a sample from the individual according to any of the methods described above, wherein a predetermined characteristic of the immune cells is indicative of a disease or a disorder in the individual. In some embodiments, the predetermined characteristic of the immune cell comprises the presence of the immune cells. In some embodiments, the presence of immune cells specifically binds to a bait composition comprising one or more neoantigens is indicative of the presence of a cancer. In some embodiments, the presence of immune cells specifically binds to a bait composition comprising one or more antigens of a pathogen (e.g., virus, bacteria, fungus) is indicative of the presence of an infection of the pathogen. In some embodiments, the presence of immune cells specifically binds to a bait composition comprising one or more autoantigens associated with an autoimmune disease or disorder is indicative of the presence of the immune disease or disorder. In some embodiments, the predetermined characteristic of the immune cell comprises a quantity of the immune cell above a threshold level. In some embodiments, the predetermined characteristic of the immune cell comprises a gene expression profile signature, a gene mutation profile signature, and/or an epigenetic modification signature. In some embodiments, the signature epigenetic modification comprises a DNA or RNA methylation, hydroxylation signature and a histone acetylation, methylation and/or glycosylation signature.

The present application in another aspect provides methods of detecting residual cancer in an individual, wherein the individual has been previously treated with an anti-cancer therapy and exhibits no pathological symptom of cancer after treatment, the method comprising analyzing a post-treatment sample from the individual according to any of the methods describe above, wherein a predetermined characteristic of the immune cell from the post-treatment sample is indicative of residual cancer in the individual. In some embodiments, the method comprises: a) analyzing a pre-treatment sample from the individual prior to anti-cancer therapy and a post-treatment sample from the individual according to the method of any of the methods described above, and b) comparing the characteristics of the immune cells from the pre-treatment sample and immune cells from the post-treatment sample; wherein a predetermined difference in characteristics of the immune cell from the pre-treatment sample and the immune cell from the post-treatment sample is indicative of residual cancer in the individual.

The present application in another aspect provides methods of treating a cancer in an individual, comprising a) diagnosing the individual as having cancer according to the method of some of the methods described above; and b) subjecting the individual to an anti-cancer therapy. In some embodiments, the anti-cancer therapy is not an immunotherapy. In some embodiments, the cancer is a solid tumor.

The present application in another aspect provides methods of treating an infection in an individual, comprising a) diagnosing the individual as having an infection by a pathogen according to some of the methods described above; and b) subjecting the individual to a therapy for the pathogen.

The present application in another aspect provides methods of treating an autoimmune disease or disorder in an individual, comprising a) diagnosing the individual as having an autoimmune disease or disorder according to some of the methods described above; and b) subjecting the individual to a therapy for the disease or disorder.

The present application in another aspect provides a composition comprising one or more display moieties comprising a particle associated with a plurality of MHC-peptide complexes, wherein the plurality of MHC-peptide complexes comprises no less than or more than 2, 3, 4, 5, 6, 8, 9, 10, or 12 MHC-peptide complexes, wherein each of the MHC-peptide complex comprises at least one MHC molecule complexed with a peptide, wherein at least two or more of the MHC-peptide complexes are different.

In some embodiments according to any of the display moieties described above, the one or more of the plurality of MHC-peptide complexes is an MHC-peptide monomer, MHC-peptide dimer, an MHC-peptide tetramer, an MHC-peptide pentamer, an MHC-peptide octamer, an MHC-peptide dextramer, or an MHC-peptide dodecamer. In some embodiments, each of the plurality of MHC-peptide complex is an MHC-peptide monomer, an MHC-peptide dimer or an MHC-peptide trimer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments according to any of the display moieties described above, the plurality of MHC-peptide complexes on the particle comprise at least 20 MHC-peptide complexes, such as at least 50 or 100 MHC-peptide complexes.

In some embodiments according to any of the display moieties described above, the MHC-peptide complex is attached to the particle via a binding pair comprising a first binding component attached to MHC-peptide complex or a unit thereof (i.e., a MHC-peptide monomer) and a second binding component bound to the particle. In some embodiments, the first binding component comprises a biotin, and the second binding component comprises streptavidin. In some embodiments, the second binding component comprises a biotin, and the first binding component comprises streptavidin.

In some embodiments according to any of the display moieties described above, the particle is solid.

In some embodiments according to any of the display moieties described above, the particle has a diameter of at least 0.1, 0.2, 0.25, 0.3, 0.4, 0.5, 0.6, 0.7, 0.8, 0.9, or 1 μm. In some embodiments, the particle has a diameter of about 0.5 μm to about 20 μm, such as about 0.5 μm to about 10 μm, or about 1 μm.

In some embodiments according to any of the display moieties described above, the particle has a surface area of about 1 μm² to about 20 μm².

In some embodiments according to any of the display moieties described above, the particle has about 10 to about 10000 second binding component associated with it.

In some embodiments according to any of the display moieties described above, the particle is spherical. In some embodiments, the particle is a bead.

In some embodiments according to any of the display moieties described above, the particle is magnetic.

In some embodiments according to any of the display moieties described above, the display moiety comprises at least two different MHC molecules. In some embodiments, the at least two different MHC molecules comprise two different MHC class I molecules. In some embodiments, the at least two different MHC molecules comprise two different MHC class II molecules. In some embodiments, the at least two different MHC molecules comprise both an MHC class I molecule and an MHC class II molecule.

In some embodiments according to any of the display moieties described above, the display moiety comprises an HLA-A molecule. In some embodiments, the HLA-A molecule comprises a mutation that reduces its binding to CD8. In some embodiments, the HLA-A molecule comprises an HLA A2 heavy chain with a A245V mutation.

In some embodiments according to any of the display moieties described above, the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03.

In some embodiments according to any of the display moieties described above, the display moiety comprises an MHC class II molecule selected from the group consisting of HLA-DQ and HLA-DR.

In some embodiments according to any of the display moieties described above, the peptide is associated with a cancer or tumor antigen. In some embodiments, the peptide is associated with a neoantigen.

In some embodiments according to any of the display moieties described above, the peptide is associated with an antigen of a pathogen. In some embodiments, the pathogen is a virus, bacteria of fungus.

In some embodiments according to any of the display moieties described above, the peptide is associated with an autoantigen.

In some embodiments according to any of the display moieties described above, the display moiety comprises at least two distinct peptides. In some embodiments, the display moiety comprises at least five distinct peptides. In some embodiments, the display moiety comprises at least 10, 20, or 50 distinct peptides. In some embodiments, the at least two distinct peptides in the display moiety do not overlap. In some embodiments, the at least two distinct peptides in the display moiety overlaps. In some embodiments, the at least two distinct peptides are associated with (e.g., derived from) two different antigens. In some embodiments, the two distinct antigens comprise two distinct mutations of one or more genes associated with a disease. In some embodiments, the disease is a cancer, the gene is a oncogene. In some embodiments, the disease is not a cancer or tumor (e.g., an infection, e.g., an autoimmune disease).

In some embodiments according to any of the display moieties described above, the display moiety does not comprise a fluorochrome or fluorophore.

The present application in another aspect provides bait compositions comprising one or more display moiety described above. In some embodiments, the bait composition comprises a plurality of the display moieties described above. In some embodiments, the bait composition comprises at least two different display moieties. In some embodiments, the bait composition comprises an HLA-A molecule, an HLA-B molecule, and an HLA-C molecule. In some embodiments, the bait composition comprises HLA-A*24: 02, HLA-A*11: 01, HLA-A*02: 01, and HLA-A*03: 01. In some embodiments, the bait composition comprises an HLA-DP molecule, an HLA-DQ molecule, and an HLA-DR molecule. In some embodiments, the bait composition comprises at least 25, 50, 100, 150, 200, or 250 different peptides. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the peptides in the bait composition are associated with mutations in at least two, three, four, five or six genes. In some embodiments, the genes are oncogenes. In some embodiments, the bait composition does not comprise a fluorochrome or fluorophore.

The present application in another aspect provides methods of analyzing immune cells in a sample from an individual, comprising a) contacting a plurality of immune cells with one or more of the display moieties or any of the bait compositions described herein, and b) detecting or analyzing the one or more immune cells that bind to one or more of the display moieties. In some embodiments, the immune cells comprise T cells. In some embodiments, the immune cells are selected from the group consisting of: cytotoxic T cells, memory T cells, and tumor infiltrating T cells. In some embodiments, the T cells are CD4+ T cells and/or CD8+ T cells. In some embodiments, the immune cells comprise B cells. In some embodiments, the immune cells comprise a mixture of immune cells comprising T cells, B cells, macrophage cells, or dendritic cells, or combinations thereof. In some embodiments, the sample is selected from the group consisting of: blood, plasma, and a peripheral blood mononuclear cell (PMBC) sample. In some embodiments, the sample is obtained from a diseased tissue. In some embodiments, the diseased tissue is a cancer tissue or tumor tissue. In some embodiments, the sample is obtained from a single individual. In some embodiments, the sample is a mixture a plurality of individual samples, each obtained from a different individual. In some embodiments, the individual is a human. In some embodiments, the individual has no indication of a disease. In some embodiments, detecting or analyzing the immune cells that bind to one or more display moieties comprises observing the agglutination of immune cells and MHC-multimer units. In some embodiments, detecting or analyzing the immune cells that bind to one or more display moieties comprises 1) immobilizing immune cells on a solid substrate prior to or after step a), 2) contacting the immobilized immune cells with a detection agent recognizing the display moieties under a condition sufficient for the agent to bind to the display moieties; and 3) detecting the detection agent, thereby detecting the immune cells bound to the display moieties.

### BRIEF DESCRIPTION OF DRAWINGS

FIG. 1 exemplifies newly predicated Kras neoantigens (SEQ ID NOs: 57-76) originated from the six most common Kras mutation (Kras G12V, Kras G12D, Kras G12R, Kras G12C, Kras G12I, Kras G12A).

FIG. 2 shows that four most common HLA-A, including HLA-A: 0201, HLA-A: 2402, HLA-A: 0301, HLA-A: 1101, and β2m were expressed and purified from E coli. The target proteins were expressed in insoluble inclusion body (DPE).

FIG. 3 shows that the assembly of five common mutant Kras neoantigen library. Four types HLA (HLA-A: 0201, HLA-A: 2402, HLA-A: 0301, HLA-A: 1101) and four most common Kras mutation (Kras G12V, Kras G12D, Kras G12R, Kras G12C). The figure only presented part of the results of Kras G12V mutation.

FIG. 4 demonstrates the confirmation of luciferase intensity in constructed pancreatic tumor cell lines. D-luciferin was used as substrate. The signal was captured 15 min later after D-luciferin intraperitoneal injection.

FIG. 5 depicts tumor sizes 12 days post inoculation of 1×10⁵ or 1×10⁶ Pano2-Luc-GFP tumor cells (panel A) and tumor growth curve of Pano2-Luc-GFP tumor in C57BL/6J mice after 1×10⁶ tumor cells were subcutaneously challenged as measured by tumor volume (panel B).

FIG. 6 depicts construction of plasmid overexpression five common Kras mutation. The band size was about 750 bp. Two replicates were loaded, and the bands less than 50 bp were non-specific bands.

FIGS. 7A and 7B depicts the detection of Kras mutation neoantigen specific CD8⁺ T cells in peripheral blood of mice as early as Day 4 after intravenous tumor cell challenge of 4×10⁵ or 1×10⁶ cells.

FIG. 8 depicts the detection of Kras mutation neoantigen specific CD8⁺ T cells in peripheral blood of mice as early as Day 4 after intravenous tumor cell challenge of 1×10⁴ or 1×10⁵ cells.

FIGS. 9A and 9B depicts the detection of Kras mutation neoantigen specific CD8⁺ T cells in peripheral blood of mice as early as Day 4 after subcutaneous tumor challenge of 4×10⁵ cells. As shown, the Tetramer⁺CD8⁺ T cells population (ranged from 0.042% to 0.22, median: 0.0866%) was detectable in mice challenged with Pan02-n peptide, but no Tetramer⁺CD8⁺ T cells were detectable in mice challenge with Pan02-EV cells.

FIGS. 10A and 10B depicts the tumor growth curve of mice inoculated with 4×10⁵ cells Pan02-EV (without neoantigen expressing Kras mutation) and Pan02-n (with neoantigen expressing Kras mutation) tumor cells as assessed via bioluminescence (FIG. 9A) and the picture of mice at day 4 post tumor challenge evidencing no observation of tumor at the site of inoculation (red circle) (FIG. 9B).

FIGS. 11A-11C depict the presence or absence of Kras mutation neoantigen specific CD8⁺ T cells with Kras mutation specific Tetramer library in peripheral blood of pancreatic cancer patients (Patient #1-13).

FIG. 12 depicts of a summary of the presence or absence of CD8⁺ T cells in thirteen confirmed pancreatic patients with a presence or absence of a Kras mutation and a specific HLA-A phenotype. In this experiment, twenty G12D neoantigen peptides associated MHC tetramers were included in the bait composition but only four G12R neoantigen peptides associated tetramers were included.

FIG. 13 depicts schematic and process of MHC Class I Tetramer (MHC-Tetramer) synthesis. (A) The components and assembly of MHC peptide monomer. (B) Schematic of MHC-Tetramer and targeting strategies for the MHC-Tetramer which binds with the specific CD8+ T cells in cancer patients blood. The exemplary targets are B2M, Streptavidin, Biotin and any labelled tags such as PE, APC or His.

FIG. 14 depicts the main process for a sandwich ELISA. (A) The method is stepwise in the order shown. The 1st step is to coat the ELISA plate with capture antibody. Then any antigen in the sample (e.g. blood, or cell supernatant) will bind to the capture antibody. Any excess sample is washed from the plate. In step 3, the detection antibody which is labelled with horse radish peroxidase or alkaline phosphatase will be added and binds to any target antigen already bound to the capture antibody. Finally, a substrate is added to the plate such as the chromogenic to convert the substrate (e.g. TMB or ABTS) into a colored product which can be measured using a plate reader. (B) The detailed protocol for a ELISA is outlined in the figure. (C) Exemplary methods. The MHC-tetramer bind to the specific TCR in the CD8+ T cells in patient blood or cells and then the CD8+ cells are captured by the CD8 antibody coated to plate or conjugated to magnetic beads. The HRP-detection antibody can identify the captured CD8 antibody using a plate reader. Alternatively, the cells are incubated with CD8 antibody coated to plate or conjugated to magnetic beads and then the MHC-tetramer are added in the resulted cells. Then the HRP-detection antibody (mainly targeting the components of MHC-Tetramer) are added and the MHC-Tetramer binding CD8+ T cells can be detected.

FIG. 15 depicts schematic and process of exemplary method to capture the specific MHG-Tetramer binding CD8+ T cells. (A-B) For example, the MHC-Tetramers with different tags are added to the immune cells first. Then the magnetic beads are added as indicated in the step 2. The presence of MHC-Tetramers binding CD8+T cells will be identified by the HRP antibodies in step 3. (C) The CD8 antibody conjugated magnetic beads are added to the immune cells first. The MHC-Tetramers with different tags are then added as indicated in the step 2. The presence of MHC-Tetramers binding CD8+T cells will be identified by the HRP antibodies in step 3.

FIG. 16 depicts strategy of clinical sample collection and tumor neoantigen specific CD8+ T cells identification by ELISA.

FIG. 17 depicts the main process and result for the detection of MHC-Tetramers binding CD8+ T cells by HRP-β2-microglobulin antibody. (A) The main method is shown stepwise. The cells were mixed with MHC-Tetramer and then incubated with CD8 antibody conjugated magnetic beads. HRP-2-microglobulin antibody was the detection antibody used to identify MHC-Tetramers binding CD8+T cells. (B) The result using the method described in (A) to detect the MHC-Tetramers binding CD8+T cells.

FIG. 18 depicts the main process and result for the detection of MHC-Tetramers binding CD8+ T cells by HRP-PE antibody. (A) The main method is shown stepwise. The cells were mixed with CD8 antibody conjugated magnetic beads firstly then incubated with MHC-Tetramers. HRP-PE antibody was the detection antibody used to identify MHC-Tetramers binding CD8+T cells. (B) The result according to method shown in (A).

FIG. 19 depicts the main process and result for the detection of MHC-Tetramers binding CD8+ T cells by HRP-PE antibody. (A) The modified method to capture the MHC-Tetramers binding CD8+ T cells by HRP-PE antibody. In this protocol, the tubes were changed to new tubes after MHC-Tetramer washing step. The tube change can also be done when CD8 conjugated bead washing step. (B) The result from this modified method shown in (A). There's a 3-fold difference between Group 1 and 4. The presentative photo for result (B). in duplicates.

FIG. 20 depicts a scheme of the exemplary method.

FIG. 21 depicts a scheme of high throughput identification of pancreatic neoantigen specific immune cells (e.g., CD 8+ T Cells) in population.

FIG. 22 depicts exemplary list of HLA subtype and high frequency tumor mutations employed in Tetramer library construction for the analysis of early pancreatic cancer

FIG. 23 depicts a scheme of DNA barcoded antibody for CD45 or CD8 specific T cells.

FIG. 24 depicts a scheme of exemplary methods for DNA barcoded CD8+ T cells identification from multiple pancreatic cancer patients by ELISA.

FIG. 25 depicts a scheme of another exemplary methods for antigen specific immune cells (e.g., neoantigen specific CD8+ T cells) capture by magnetic beads and DNA barcode based ELISA assay.

FIG. 26 depicts a scheme of another exemplary methods for antigen specific immune cells (e.g., neoantigen specific CD8+ T cells) capture by tetramer-beads and DNA barcode based ELISA assay.

FIG. 27A depicts a scheme of another exemplary methods for antigen specific immune cells (e.g., neoantigen specific CD8+ T cells) capture by MHC-tetramer-beads bound with multiple antigenic peptides and DNA barcode based ELISA assay.

FIG. 27B depicts a scheme of another exemplary methods for antigen specific immune cells (e.g., neoantigen specific CD8+ T cells) capture by MHC-tetramers bound with multiple antigenic peptides and DNA barcode based ELISA assay.

FIG. 27C exemplifies Kras neoantigens (SEQ ID NOs: xx-xx) originated from Kras mutations (Kras G12V, Kras G12D, Kras G12R).

FIG. 28A depicts the characterization of MHC-tetramer and MHC-tetramer-bead on PAGE.

FIG. 28B depicts the detection of Kras mutation neoantigen specific CD8⁺ T cells in peripheral blood of mice by MHC-tetramer-beads in an agglutination assay.

FIG. 28C depicts the results of Kras mutation neoantigen specific CD8⁺ T cells in peripheral blood of mice by MHC-tetramer-beads in an agglutination assay.

FIG. 28D depicts the detection of Kras mutation neoantigen specific CD8⁺ T cells in mice spleen by MHC-tetramer-beads in an agglutination assay.

FIG. 28E depicts the results of Kras mutation neoantigen specific CD8⁺ T cells in mice spleen by MHC-tetramer-beads in an agglutination assay.

FIG. 29A depicts the characterization of MHC-β-galactosidase-beads and Kras G12D MHC-monomer on PAGE.

FIG. 29B depicts the characterization of MHC-β-galactosidase-beads and Kras G12D MHC-monomer and MHC-tetramer on PAGE.

FIG. 29C depicts the detection of Kras G12D neoantigen specific CD8⁺ T cells in peripheral blood of mice by MHC-tetramer-beads in an agglutination assay.

FIG. 29D-29E depict the detection of Kras G12D neoantigen specific CD8⁺ T cells in mice spleen by MHC-tetramer-beads in an agglutination assay.

FIG. 30A depicts the characterization of MHC-monomer bound with multiple antigenic peptides on PAGE.

FIG. 30B depicts the characterization of MHC-tetramer bound with multiple antigenic peptides on PAGE.

FIG. 30C depicts the peptide standards in LC-MS/MS detection.

FIG. 30D depicts the detection of antigenic peptides from PAGE.

FIG. 30E depicts the characterization of MHC-tetramer bound with multiple antigenic peptides in LC-MS/MS detection.

FIG. 30F depicts the characterization of MHC-monomer and MHC-tetramer on PAGE.

FIG. 31A depicts the detection of Kras G12V neoantigen specific CD8⁺ T cells in mice spleen by MHC-monomer-beads.

FIG. 31B depicts the detection of Kras G12D neoantigen specific CD8⁺ T cells in human peripheral blood by MHC-monomer-beads.

FIG. 31C depict the detection of Kras multiple specific CD8⁺ T cells in human peripheral blood by MHC-monomer-beads with multiple antigenic peptides.

FIG. 31D-FIG. 31E depict the detection of stimulated Kras multiple specific CD8⁺ T cells in human peripheral blood by MHC-monomer-beads with multiple antigenic peptides.

### DETAILED DESCRIPTION

The present application provides methods of analyzing a sample from one or more individuals for the presence or absence of immune cells capable of binding to an antigenic peptide. In some embodiments, the presence such immune cells are indicative of the presence of a disease or condition in the individual. The application is at least partly based on inventors' unique insight that immune cells activated by specific antigens (such as tumor neoantigens) can be detectable in an individual even before any pathological symptoms of disease or condition (e.g., cancer) are exhibited, thus serving as useful markers for detection of such disease or condition at early stage.

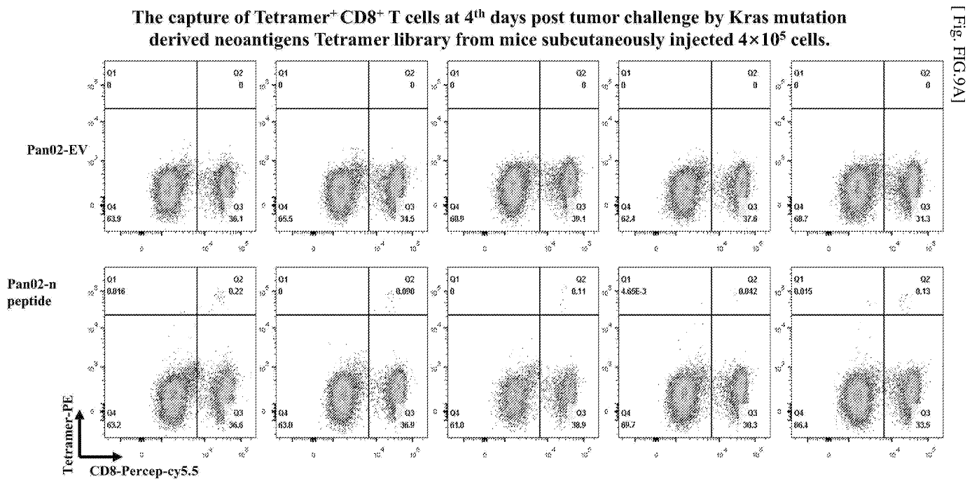

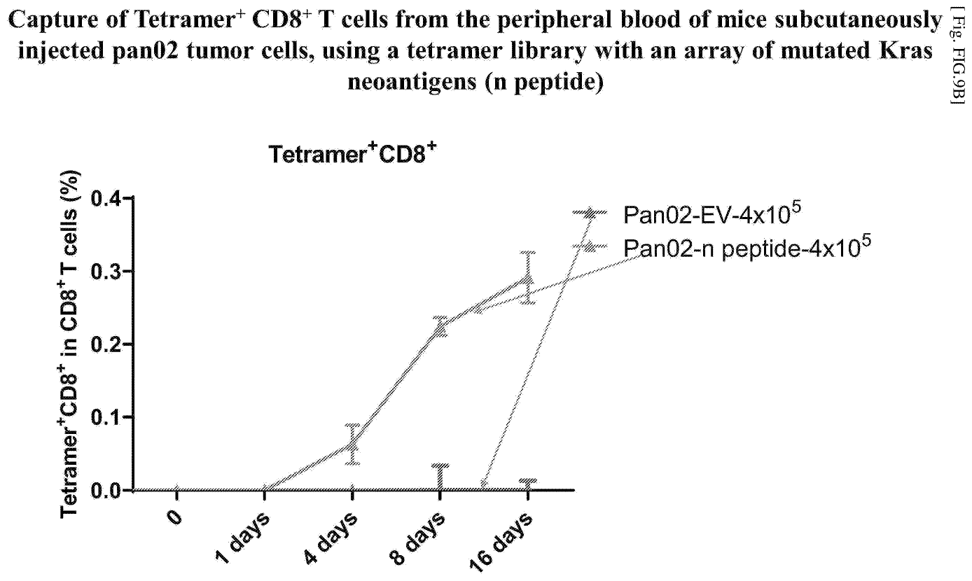

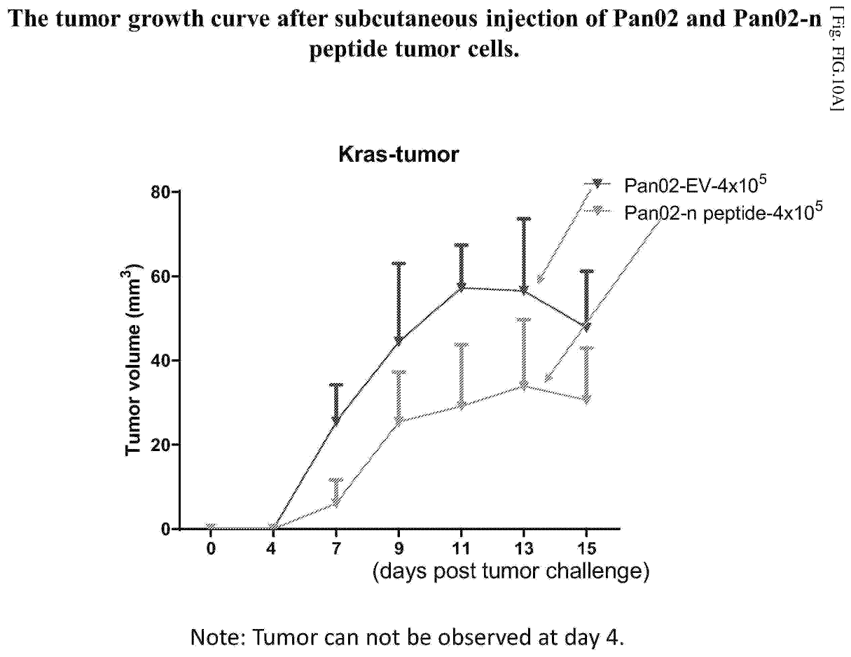

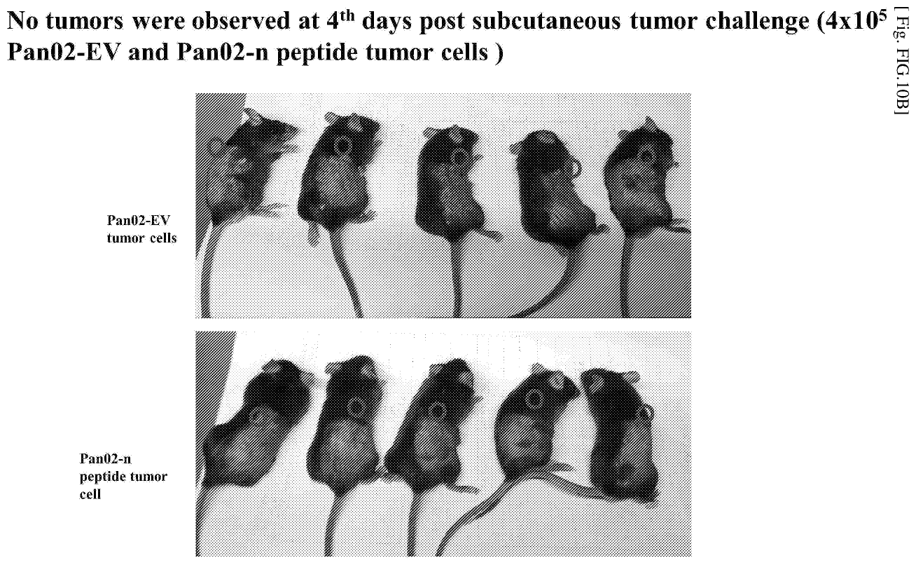

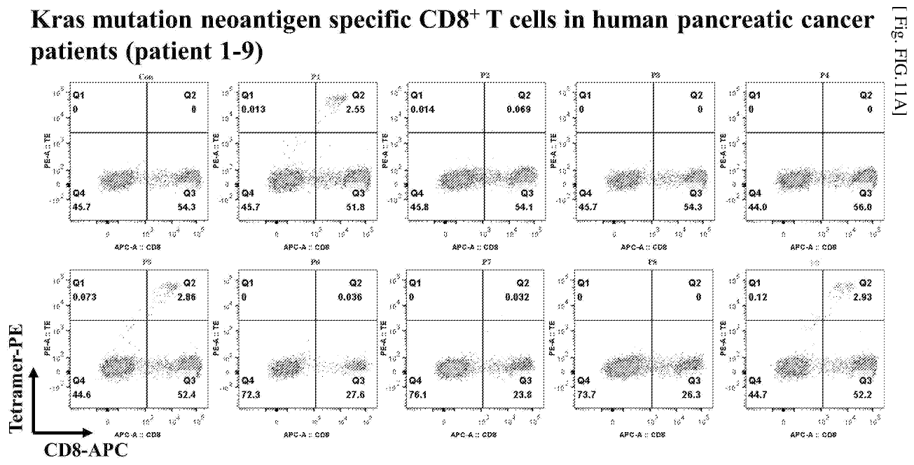

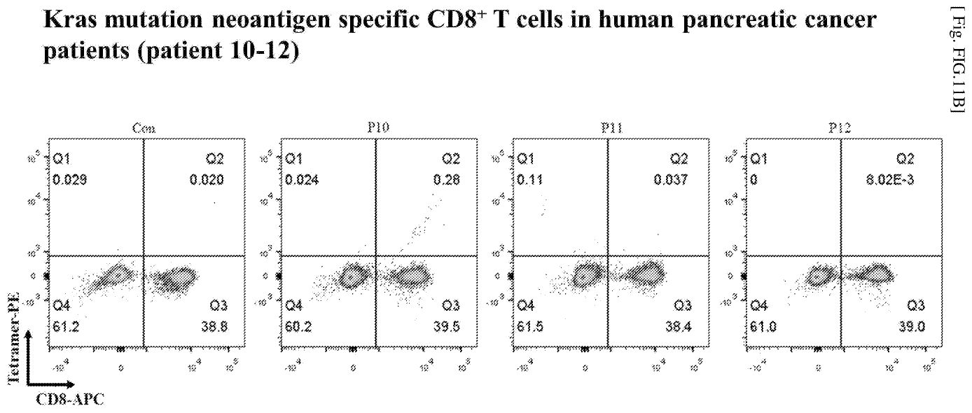

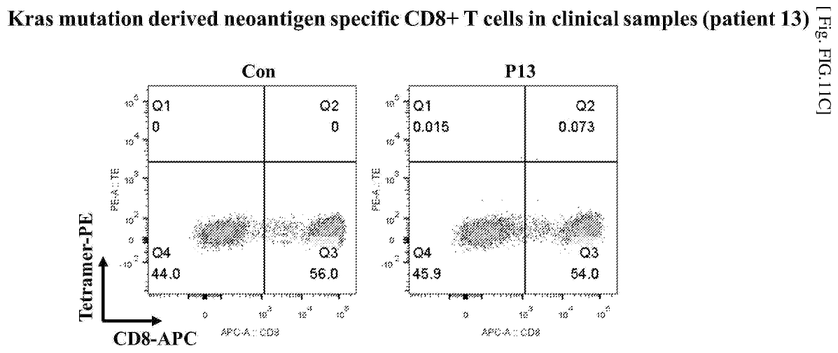

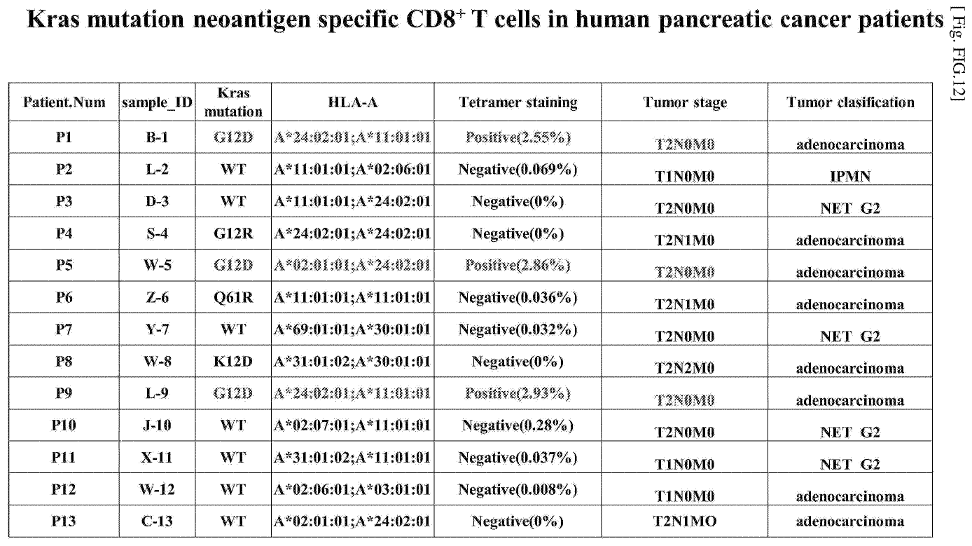

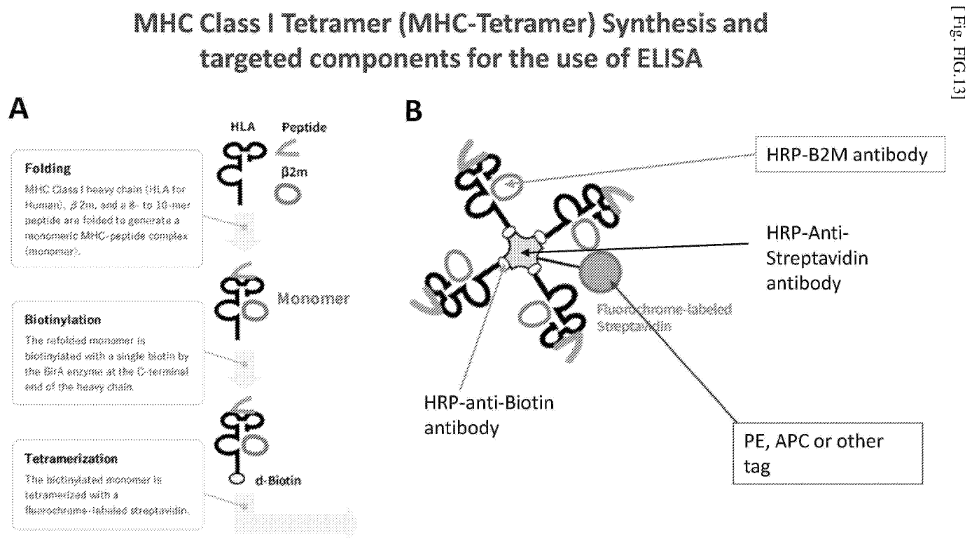

As shown in Example section, exemplary Kras mutation associated neoantigen-specific T cells were successfully detected in mice intravenously or subcutaneously inoculated with tumor cells (of as low as 10⁴ cells, see, e.g., FIG. 8) expressing Kras mutation associated neoantigens and as early as day 4 after inoculation (see e.g., FIG. 9A-9B), which is prior to the any detection via bioluminescence (see e.g., FIG. 10A-10B), or in pancreatic cancer patients harboring Kras mutations (e.g., see FIGS. 11A-11C and 12). These data demonstrated that the provided methods can be successfully used for cancer detection including both early cancer detection as well as for monitoring cancer cells in individuals who may have residual minimal cancer cells.

The present application also provides innovative methods that exploit immunoassays such as ELISA for effectively, economically, and efficiently detect the presence of antigen-specific immune cells even when they are in a very low abundance in a sample (e.g., less than 1%). The methods of the present application have been demonstrated to successfully detect neoantigen-specific immune cells in cancer patients and detecting pathogen-specific immune cells in patients infected with the pathogen. Some embodiments of the methods demonstrate surprisingly high sensitivity. Moreover, the methods enable a high throughput analysis of a mixed sample that has immune cells derived from a plurality of individuals (e.g., 10-30 individuals) by associating a unique barcode with the immune cells from each individuals. See e.g., Examples 3 and 4.

The present application in one aspect provides methods of analyzing a sample from one or more individuals for the presence or absence of immune cells capable of binding to an antigenic peptide, the method comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety.

The present application in another aspect provides methods of analyzing a sample from one or more individuals for the presence or absence of immune cells capable of binding to an antigenic peptide, the method comprising: a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety.

The present application in another aspect provides methods of analyzing a plurality of samples from one or more individuals for the presence or absence of immune cells capable of binding to an antigenic peptide, comprising: a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived.

Methods of detecting antigen-specific immune cells in an individual, methods of detecting residual cancer, methods of treating a cancer, infection or an autoimmune disease are also provided.

The present application in another aspect provides compositions comprising display moieties comprising a particle associated with a plurality of MHC-peptide complexes. In some embodiments, the plurality of MHC-peptide complexes comprises more than 2, 3, 4, 5, 6, 8, 9, 10 or 12 MHC-peptide complexes, wherein each of the MHC-peptide complex comprises at least one MHC molecule complexed with a peptide, wherein at least two or more of the MHC-peptide complexes are different. In some embodiments, the MHC-peptide complex is an MHC-peptide monomer. In some embodiments, the particle is solid. In some embodiments, the plurality of MHC-peptide complexes comprises at least 20, 50, or 100 MHC-peptide complexes.

As demonstrated in e.g., the examples, the display moieties described herein are a powerful tool that can be used to simultaneously screen antigen-specific immune cells from an individual that specifically recognize a plurality of peptides (e.g., neoantigen peptides) with high sensitivity. These display moieties, by having a plurality of heterogenous MHC-peptide complexes associated with a particle, enable aggregation of antigen-specific immune cells that can be conveniently observed under microscope or bare eyes. Accordingly, it provides powerful, sensitive, cost-effective and convenient platform technology for screening, detecting and analyzing antigen-specific immune cells in different contexts (e.g., cancers, autoimmune diseases, infections).

Bait compositions comprising the display moieties, methods of analyzing or detecting antigen-specific immune cells by using the display moieties or bait compositions (such as those described above), and methods of treating patients that involve using methods of detecting antigen-specific immune cells as described herein are also provided in the application.

### DEFINITIONS

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response.

As used herein the term “neoantigen” is an antigen that has at least one alteration that makes it distinct from the corresponding wild-type, parental antigen, e.g., via mutation in a tumor cell or post-translational modification specific to a tumor cell. A neoantigen can include a polypeptide sequence. A mutation that results in a neoantigen can include a frameshift or non-frameshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354 (6310): 354-358.

As used herein the term “tumor neoantigen” or “cancer neoantigen” is a neoantigen present in a subject's tumor cell or tissue but not in the subject's corresponding normal cell or tissue.

As used herein the term “missense mutation” is a mutation causing a substitution from one amino acid to another.

As used herein the term “nonsense mutation” is a mutation causing a substitution from an amino acid to a stop codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

As used herein the term “epitope” is the specific portion of an antigen typically bound by an antibody or T-cell receptor.

As used herein the term “immunogenic” is the ability to elicit an immune response, e.g., via T-cells, B cells, or both.

As used herein the term “HLA binding affinity” “MHC binding affinity” means affinity of binding between a specific antigen and a specific MHC allele.

As used herein the term “bait composition” is a composition comprising a molecule (e.g., an antigenic peptide) used to enrich a cell that specifically binds to the bait from a sample. To clarify, “antigenic peptide” described herein is not limited to peptides that are capable of inducing an immune response. It is exchangeable to “peptide” and includes any peptide that can be presented by an MHC molecule.

As used herein the term “variant” is a difference between a subject's nucleic acids and the reference human genome used as a control.

As used herein the term “allele” is a version of a gene or a version of a genetic sequence or a version of a protein.

As used herein the term “HLA type” is the complement of HLA gene alleles.

As used herein the term “exome” is a subset of the genome that codes for proteins. An exome can be the collective exons of a genome.

As used herein the term “proteome” is the set of all proteins expressed and/or translated by a cell, group of cells, or individual.

As used herein the term “dextramers” is a dextran-based peptide-MHC multimers used for antigen-specific immune-cell staining in flow cytometry.

As used herein the term “MHC multimers” is a peptide-MHC complex comprising multiple peptide-MHC monomer units.

As used herein the term “MHC tetramers” is a peptide-MHC complex comprising four peptide-MHC monomer units. As used herein the term “MHC monomers” is a peptide-MHC complex comprising one peptide-MHC monomer units. As used herein the term “MHC dimers” is a peptide-MHC complex comprising two peptide-MHC monomer units. As used herein the term “MHC trimers” is a peptide-MHC complex comprising three peptide-MHC monomer units.

As used herein the term “Latex Agglutination Test (LAT)” refers to a class of bioanalytical methods used to detect the presence (qualitive) and sometimes the amount of a substance (quantitative) in a sample. In the presence of a specific substance (e.g. antigen, antibody, or cell), the latex particles agglomerate, forming clumps or aggregations of particles.

As used herein the term “Latex Beads” refers to spherical particles, e.g. polystyrene, poly (methyl methacrylate), polyacrylates, or other similar types of polymers. These Latex beads can vary in size, typically ranging from nanometers to micrometers in diameter. Their surface may be functionalized or modified to allow for the attachment of biological molecules, chemical compounds, or other substances. Latex beads can be used in a range of applications including, but not limited to, diagnostic assays, bioseparation, drug delivery, and as standards or controls in various analytical techniques.

As used herein, “sample” refers to an aliquot of body fluid or a tissue obtained from a subject which contains an immune cell.

The term “mammal” encompasses both humans and non-humans and includes but is not limited to humans, non-human primates, canines, felines, murines, bovines, equines, and porcines.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

The terms “subject,” “individual,” and “patient” are used interchangeably herein to refer to a mammal, including, but not limited to, human, bovine, horse, feline, canine, rodent, or primate. In some embodiments, the individual is a human.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

It should be noted that, as used in the specification and t e appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

Methods of Analyzing a Sample from One or More Individuals

The present application provides methods of analyzing a sample from one or more individuals, e.g., for the presence or absence of immune cells capable of binding to an antigenic peptide. Such methods can be further used for e.g., diagnosis of a disease or condition (e.g., a cancer, e.g., an infection, e.g., an autoimmune cells).

In some embodiments, there is provided a method of analyzing a plurality of samples from one or more individuals for the presence or absence of immune cells capable of binding to an antigenic peptide comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety, wherein the antigenic peptide is associated with a cancer or tumor antigen, optionally wherein the antigenic peptide is a neoantigen peptide. In some embodiments, the sample is obtained from one or more individuals exhibiting no pathological symptom of a cancer. In some embodiments, the individual a) has not previously been diagnosed as having a cancer, optionally wherein the individual is at risk of having cancer, or b) the individual has been previously treated for cancer and exhibits no pathological symptom of a cancer after the treatment. In some embodiments, the neoantigenic peptide comprising a mutation relative to a wildtype peptide, optionally at the third amino acid position counting from the N-terminus. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other, e.g., by washing the immune cells for at least 1, 2, or 3 times.

In some embodiments, there is provided a method of analyzing a plurality of samples from one or more individuals for the presence or absence of immune cells capable of binding to an antigenic peptide comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety, wherein the antigenic peptide is associated with a pathogen antigen, optionally wherein the antigenic peptide is associated with a virus, a bacteria, or a fungus antigen. In some embodiments, the individual a) has not been diagnosed as having an infection by a pathogen, optionally wherein the individual is at risk of having the infection, and/or b) exhibits no pathological symptom of an infection by a pathogen, optionally exhibits no pathological symptom of an infection by the pathogen. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety, wherein the antigenic is an autoantigenic peptide associated with an autoantigen. In some embodiments, the individual a) has not been diagnosed as having an immune response against the autoantigen, optionally wherein the individual is at risk of developing the immune response against the autoantigen, and/or b) exhibits no pathological symptom of an immune response (e.g., an autoimmune disease or disorder), optionally exhibits no pathological symptom of an immune response against the autoantigen. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety, wherein the immune cells are immobilized on the solid substrate such as a bead, and optionally wherein the bead is associated with an antibody recognizing the immune cells, optionally wherein the antibody is a CD8 antibody. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety, wherein the display moiety comprises a library of antigenic peptides for a mutation associated with a cancer or tumor, a pathogen, or an autoantigen, wherein the library comprises at least about 5, 10, 12, 15, 18 or 20 distinct antigenic peptides (e.g., neoantigenic). In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety, wherein the antigenic peptide 1) has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues, and/or 2) has low immunogenicity. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety, wherein the display moiety further comprises a detectable label (e.g., a fluorophore, e.g., a PE or APC), wherein the detection agent binds to the detectable label. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety, wherein the antigenic peptide is associated with a cancer or tumor antigen, optionally wherein the antigenic peptide is a neoantigen peptide. In some embodiments, the sample is obtained from one or more individuals exhibiting no pathological symptom of a cancer. In some embodiments, the individual a) has not previously been diagnosed as having a cancer, optionally wherein the individual is at risk of having cancer, or b) the individual has been previously treated for cancer and exhibits no pathological symptom of a cancer after the treatment. In some embodiments, the neoantigenic peptide comprising a mutation relative to a wildtype peptide, optionally at the third amino acid position counting from the N-terminus. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety, wherein the antigenic peptide is associated with a pathogen antigen, optionally wherein the antigenic peptide is associated with a virus, a bacteria, or a fungus antigen. In some embodiments, the individual a) has not been diagnosed as having an infection by a pathogen, optionally wherein the individual is at risk of having the infection, and/or b) exhibits no pathological symptom of an infection by a pathogen, optionally exhibits no pathological symptom of an infection by the pathogen. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety, wherein the antigenic is an autoantigenic peptide associated with an autoantigen. In some embodiments, the individual a) has not been diagnosed as having an immune response against the autoantigen, optionally wherein the individual is at risk of developing the immune response against the autoantigen, and/or b) exhibits no pathological symptom of an immune response (e.g., an autoimmune disease or disorder), optionally exhibits no pathological symptom of an immune response against the autoantigen. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03.

In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety, wherein the immune cells are immobilized on the solid substrate such as a bead, and optionally wherein the bead is associated with an antibody recognizing the immune cells, optionally wherein the antibody is a CD8 antibody. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety, wherein the display moiety comprises a library of antigenic peptides for a mutation associated with a cancer or tumor, a pathogen, or an autoantigen, wherein the library comprises at least about 5, 10, 12, 15, 18 or 20 distinct antigenic peptides (e.g., neoantigenic). In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety, wherein the antigenic peptide 1) has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues, and/or 2) has low immunogenicity. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety, wherein the display moiety further comprises a detectable label (e.g., a fluorophore, e.g., a PE or APC), wherein the detection agent binds to the detectable label. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived, wherein the antigenic peptide is associated with a cancer or tumor antigen, optionally wherein the antigenic peptide is a neoantigen peptide. In some embodiments, the sample is obtained from one or more individuals exhibiting no pathological symptom of a cancer. In some embodiments, the individual a) has not previously been diagnosed as having a cancer, optionally wherein the individual is at risk of having cancer, or b) the individual has been previously treated for cancer and exhibits no pathological symptom of a cancer after the treatment. In some embodiments, the neoantigenic peptide comprising a mutation relative to a wildtype peptide, optionally at the third amino acid position counting from the N-terminus. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived, wherein the antigenic peptide is associated with a pathogen antigen, optionally wherein the antigenic peptide is associated with a virus, a bacteria, or a fungus antigen. In some embodiments, the individual a) has not been diagnosed as having an infection by a pathogen, optionally wherein the individual is at risk of having the infection, and/or b) exhibits no pathological symptom of an infection by a pathogen, optionally exhibits no pathological symptom of an infection by the pathogen. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived, wherein the antigenic is an autoantigenic peptide associated with an autoantigen. In some embodiments, the individual a) has not been diagnosed as having an immune response against the autoantigen, optionally wherein the individual is at risk of developing the immune response against the autoantigen, and/or b) exhibits no pathological symptom of an immune response (e.g., an autoimmune disease or disorder), optionally exhibits no pathological symptom of an immune response against the autoantigen. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the immune cells are immobilized on the solid substrate such as a bead, optionally wherein the bead is associated with an antibody recognizing the immune cells, optionally wherein the antibody is a CD8 antibody. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived, wherein the immune cells are immobilized on a solid substrate (e.g., via a bead), and optionally wherein the bead is associated with an antibody recognizing the immune cells, optionally wherein the antibody is a CD8 antibody. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises two or more antigenic peptides, optionally wherein the display moiety comprises four antigenic peptides. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived, wherein the display moiety comprises a library of antigenic peptides for a mutation associated with a cancer or tumor, a pathogen, or an autoantigen, wherein the library comprises at least about 5, 10, 12, 15, 18 or 20 distinct antigenic peptides (e.g., neoantigenic). In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the antigenic peptide has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues. In some embodiments, the antigenic peptide has low immunogenicity. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived, wherein the antigenic peptide 1) has one or more of the following characteristics: a) having a binding affinity of about 1 nM to about 5000 nM to an MHC molecule; b) having a binding affinity of about 1 nM to about 5000 nM to a cognate TCR molecule; c) is hydrophobic; and d) has high content of aromatic residues, and/or 2) has low immunogenicity. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety further comprises a detectable label (e.g., a fluorophore). In some embodiments, the method further comprises isolating immune cells bound to the display moiety or removing the display moiety or immune cells not bound to each other.

In some embodiments, there is provided a method of analyzing a plurality of samples from one or more individuals for the presence or absence of immune cells capable of binding to an antigenic peptide comprising a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived, wherein the display moiety further comprises a detectable label (e.g., a fluorophore, e.g., a PE or APC), wherein the detection agent binds to the detectable label. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization. In some embodiments, the sample is a blood sample (e.g., PBMC sample). In some embodiments, the immune cells comprise T cells (e.g., CD8 T cells), optionally the immune cells comprise no more than about 10%, 5%, 4%, 3%, 2% or 1% T cells specifically binding to antigenic peptide in the bait composition. In some embodiments, the display moiety comprises an MHC molecule complexed with the antigenic peptide, optionally wherein the MHC molecule comprises a MHC multimer, further optionally the MHC multimer is a MHC tetramer or a MHC dextramer. In some embodiments, the MHC molecule comprises a MHC class I molecule. In some embodiments, the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation. In some embodiments, the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the method further comprises isolating immune cells bound to the display moiety or removing the display moiety or immune cells not bound to each other.

In some embodiments, the “condition” in the term “under a condition sufficient for an immune cell to bind to the display moiety” refers to conditions in which specific binding between the immune cells and the display moiety happen. In some embodiments, it refers to conditions that involve about or at least about 5, 10, 15, 20, 25, or 30 minutes of incubation of the immune cells and display moiety (e.g., at room temperature in a medium). In some embodiments, it refers to conditions under at least 10%, 20%, 30%, 40%, 50%, 60%, 70%, 80%, 90% of the maximum binding between the immune cells and the display moiety happen. Maximum binding can be assessed by, e.g., contacting immune cells with display moiety for adequate time (e.g., 30 minutes) under a proper condition (e.g., room temperature).

In some embodiments, the immune cells are immobilized to a solid substrate, e.g., via an agent binding to the immune cells that is coated to or conjugated with the solid substrate. In some embodiments, the agent is an antibody that specifically recognizes a surface marker of the immune cells. In some embodiments, the agent is an anti-CD8 antibody and the immune cells are CD8 T cells. In some embodiments, the agent is an anti-CD4 antibody and the immune cells are CD4 T cells. In some embodiments, the agent is an anti-CD3 antibody and the immune cells are T cells. The immobilization can be carried out by mixing immune cells with the agent (or incubating the immune cells with the agent) that specifically binds to the immune cells. In some embodiments, the agent is already coated or conjugated on a solid substrate, such as a plate or magnetic bead. The immune cells are allowed to be contacting with the agent for a period time and then a washing step can be applied to remove the cells not bound to the agent (e.g., using a magnet). See e.g., Example 2-4.

In some embodiments, the solid support (or “solid substrate” which can be used interchangeably) comprises a plate or a surface of a particle (such as a surface of a particle, such as an inner surface of a porous particle). In some embodiments, the solid support comprises any of beads (e.g., magnetic beads), porous beads, porous matrices, arrays, glass surfaces, silicon surfaces, plastic surfaces, filters, membranes, nylons, silicon wafer chips, flow-through chips. In some embodiments, the solid support comprises any of a biochip containing electronics, a microtiter well, an ELISA plate, a spin interference disc, a nitrocellulose membrane, a nitrocellulose-based polymer surface, a nanoparticle, or a microsphere.

In some embodiments, the sample is a mixture a plurality of individual samples. In some embodiments, the plurality of individual samples are from a single individual. In some embodiments, the plurality of individual samples are from a plurality of individuals.

In some embodiments, the sample is a mixture a plurality of individual samples and each of the plurality of individual samples is obtained from a different individual. In some embodiments, the method comprises prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. The association can be achieved by e.g., mixing immune cells with a molecule (e.g., an antibody that binds to the immune cells, e.g., a CD45 antibody) that comprises the unique barcode and binds to the immune cells for them to bind to each other. For example, unique barcode can be associated to cells via e.g., an antibody conjugated or fused with the barcode that specifically binds to the cells. In some embodiments, the barcode is a nucleic acid barcode (e.g., DNA barcode). In some embodiments, the unique DNA barcode is randomized barcode with a length of about 10-20 nucleic acids (e.g., 12-18 nucleic acids). In some embodiments, the barcode is a peptide barcode. In some embodiments, the method further comprises removing unbound molecule comprising the barcode (molecules not bound by the immune cells) e.g., via washing the immune cells for at least 1, 2, or 3 times.

In some embodiments, the method further comprises determining the unique barcode associated with the immune cells bound to the display moiety, thereby identifying the sample from which the immune cells are derived. For example, nucleic acids can be purified, and sequencing can used to identify the immune cells with which a unique DNA barcode is associated with. See e.g., Example 4.

In some embodiments, the particle described herein is a dextran particle. In some embodiments, the particle is a magnetic nanoparticle or polystyrene nanoparticle. In some embodiments, the particle is an agarose bead or a sepharose bead. In some embodiments, the antigenic peptide or MHC is directly attached to the particle. In some embodiments, the antigenic peptide or MHC is attached to the particle via a binding pair comprising a first binding component attached to the antigenic peptide and a second binding component bound to the particle. In some embodiments, the display moiety comprises a cell. In some embodiments, the cell comprises a polynucleotide encoding the antigenic peptide. In some embodiments, the polynucleotide encodes a plurality of antigenic peptides.

In some embodiments, the individual is a human. In some embodiments, the individual is at least about 50 years old (e.g., at least 50, 60, 70, or 80 years old).

In some embodiments, the sample described herein (including this section and any other sections of the present application) can be any sample from the individual. In some embodiments, the sample described herein is a blood sample. In some embodiments, the sample described herein is a plasma sample. In some embodiments, the sample described herein comprises peripheral blood mononuclear cell (PMBC). In some embodiments, the sample described herein is a sample (e.g., a biopsy sample) obtained from a tissue or organ of the individual. In some embodiments, the sample is obtained from a lymph node of the individual.

The methods described herein (including methods of analyzing immune cells and methods of detecting antigen-specific immune cells described in following sections) have high specificity and sensitivity and enable it to detect antigen-specific immune cells even when they are rare in the sample (e.g., less than 1%). Sensitivity is calculated as the % of the true positive/(true positive+false negative). Specificity is calculated as the % of the true negative/(true negative+false positive). In some embodiments, the method described herein demonstrated a specificity of more than about 50%, 60%, 70%, 80%, 90%, 95%, or 100%. In some embodiments, the method described herein demonstrated a sensitivity of more than about 50%, 55%, 60%, 65%, 70%, 75% or 80%. See e.g., FIG. 12. In some embodiments, the sample comprises PBMC and the immune cells comprise or are T cells or B cells contained in the PBMC. In some embodiments, the immune cells are not subject to an enrichment step for the immune cells in general or immune cells that specifically binds to the antigenic peptide prior to contacting with the bait composition. In some embodiments, the immune cells are not subject to an enrichment step for the immune cells in general or immune cells that specifically binds to the antigenic peptide throughout this methods. In some embodiments, the sample comprise no more than 1×10⁸, 5×10⁷, 2×10⁷, 1×10⁷, 5×10⁶, 2×10⁶, or 1×10⁶ PBMCs. In some embodiments, the sample comprise no more than 1×10⁶, 5×10⁵, 2×10⁵, 1×10⁵, 5×10⁴, 2×10⁴, 1×10⁴, 7×10³, 5×10³, 2×10³, 1×10³, 5×10², 2×10², or 1×10² immune cells (e.g., CD8 T cells, e.g., immune cells that bind to antigenic peptide(s) in the bait composition, e.g., CD4 or CD8 T cells that bind to antigenic peptide(s) in the bait composition).

### Methods of Analyzing Immune Cells

The present application further comprises methods of analyzing immune cells in a sample from an individual, comprising a) contacting a plurality of immune cells with the one or more display moieties or a bait composition described herein, and b) detecting or analyzing the one or more immune cells that bind to one or more of the display moieties.

In some embodiments, there is provided a methods of analyzing immune cells in a sample from an individual, comprising a) contacting a plurality of immune cells with one or more display moieties or a bait composition described herein, and b) detecting or analyzing the one or more immune cells that bind to one or more of the display moieties, wherein the immune cells are obtained from a sample (e.g., PBMCs) of an individual, wherein the individual does not exhibit a pathological symptom of a cancer, infection or autoimmune disease. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least more than 12 MHC-peptide complexes, wherein each of the MHC-peptide complex is an MHC-peptide monomer comprising an MHC molecule complexed with a peptide, wherein at least two MHC-peptide complexes are different. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells).

In some embodiments, there is provided a methods of analyzing immune cells in a sample from an individual, comprising a) contacting a plurality of immune cells with one or more display moieties or a bait composition described herein, and b) detecting or analyzing the one or more immune cells that bind to one or more of the display moieties, wherein the immune cells are obtained from a sample (e.g., PBMCs) of an individual, wherein the individual does not exhibit a pathological symptom of a cancer, infection or autoimmune disease. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the plurality of MHC-peptide complexes comprises at least 20, 50, 100, 500, 1,000, 5,000, 10,000, 20,000, 50,000, 10,0000, 15,000, 20,0000 MHC-peptide complexes. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties or a bait composition described herein, and b) detecting or analyzing the one or more immune cells that bind to one or more of the display moieties, wherein the immune cells are obtained from a sample (e.g., PBMCs) of an individual, wherein the individual does not exhibit a pathological symptom of a cancer, infection or autoimmune disease. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the particle has a surface area of at least about 1 μm², or has a diameter of at least 0.5 μm. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties or a bait composition described herein, and b) detecting or analyzing the one or more immune cells that bind to one or more of the display moieties, wherein the immune cells are obtained from a sample (e.g., PBMCs) of an individual, wherein the individual does not exhibit a pathological symptom of a cancer, infection or autoimmune disease. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the plurality of MHC-peptide complexes comprise a) at least two different MHC molecules, and b) at least two different peptides. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties or a bait composition described herein, and b) detecting or analyzing the one or more immune cells that bind to one or more of the display moieties, wherein the immune cells are obtained from a sample (e.g., PBMCs) of an individual, wherein the individual does not exhibit a pathological symptom of a cancer, infection or autoimmune disease. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the display moiety comprises at least two different MHC class I molecules. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties or a bait composition described herein, and b) detecting or analyzing the one or more immune cells that bind to one or more of the display moieties, wherein the immune cells are obtained from a sample (e.g., PBMCs) of an individual, wherein the individual does not exhibit a pathological symptom of a cancer, infection or autoimmune disease. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the display moiety comprises at least two different MHC class II molecules. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties or a bait composition described herein, and b) detecting or analyzing the one or more immune cells that bind to one or more of the display moieties, wherein the immune cells are obtained from a sample (e.g., PBMCs) of an individual, wherein the individual is at risk of developing a disease or condition, such as cancer, infection or autoimmune disease or disorder, e.g., an individual who has a family history of such disease or has been exposed to a pathogen, but has not been treated for the disease or condition. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells).

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual is at risk of developing a disease or condition, such as cancer, infection or autoimmune disease or disorder, e.g., an individual who has a family history of such disease or has been exposed to a pathogen, but has not been treated for the disease or condition. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex is an MHC-peptide monomer comprising an MHC molecule complexed with a peptide, wherein at least two MHC-peptide complexes are different. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells).

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual is at risk of developing a disease or condition, such as cancer, infection or autoimmune disease or disorder, e.g., an individual who has a family history of such disease or has been exposed to a pathogen, but has not been treated for the disease or condition. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the plurality of MHC-peptide complexes comprises at least 20, 50, 100, 500, 1,000, 5,000, 10,000, 20,000, 50,000, 10,0000, 15,000, 20,0000 MHC-peptide complexes. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual is at risk of developing a disease or condition, such as cancer, infection or autoimmune disease or disorder, e.g., an individual who has a family history of such disease or has been exposed to a pathogen, but has not been treated for the disease or condition. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the particle has a surface area of at least about 1 μm², or has a diameter of at least 0.5 μm. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual is at risk of developing a disease or condition, such as cancer, infection or autoimmune disease or disorder, e.g., an individual who has a family history of such disease or has been exposed to a pathogen, but has not been treated for the disease or condition. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the plurality of MHC-peptide complexes comprise a) at least two different MHC molecules, and b) at least two different peptides. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual is at risk of developing a disease or condition, such as cancer, infection or autoimmune disease or disorder, e.g., an individual who has a family history of such disease or has been exposed to a pathogen, but has not been treated for the disease or condition. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the display moiety comprises at least two different MHC class I molecules. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual is at risk of developing a disease or condition, such as cancer, infection or autoimmune disease or disorder, e.g., an individual who has a family history of such disease or has been exposed to a pathogen, but has not been treated for the disease or condition. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the display moiety comprises at least two different MHC class II molecules. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties or a bait composition described herein, and b) detecting or analyzing the one or more immune cells that bind to one or more of the display moieties, wherein the immune cells are obtained from a sample (e.g., PBMCs) of an individual, wherein the individual has minimal residual disease (MRD). In some embodiments, the individual has minimal residual cancer. In some embodiments, the minimal residual cancer is seen after the cancer was surgical resected or cured. In some embodiments, the minimal residual disease is too minimal to be detected by imaging instruments (e.g., a routinely used or standard imaging instrument used for detection of the cancer). In some embodiments, the location of the minimal residual disease is diverse. In some embodiments, the minimal residual cancer is a result of immune escape or resistance to treatment. In some embodiments, the individual has been previously treated for cancer and exhibits no pathological symptom of a cancer after the treatment. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual has minimal residual disease (MRD). In some embodiments, the individual has minimal residual cancer. In some embodiments, the minimal residual cancer is seen after the cancer was surgical resected or cured. In some embodiments, the individual has been previously treated for cancer and exhibits no pathological symptom of a cancer after the treatment. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex is an MHC-peptide monomer comprising an MHC molecule complexed with a peptide, wherein at least two MHC-peptide complexes are different. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells).

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual has minimal residual disease (MRD). In some embodiments, the individual has minimal residual cancer. In some embodiments, the minimal residual cancer is seen after the cancer was surgical resected or cured. In some embodiments, the individual has been previously treated for cancer and exhibits no pathological symptom of a cancer after the treatment. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the plurality of MHC-peptide complexes comprises at least 20, 50, 100, 500, 1,000, 5,000, 10,000, 20,000, 50,000, 10,0000, 15,000, 20,0000 MHC-peptide complexes. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual has minimal residual disease (MRD). In some embodiments, the individual has minimal residual cancer. In some embodiments, the minimal residual cancer is seen after the cancer was surgical resected or cured. In some embodiments, the individual has been previously treated for cancer and exhibits no pathological symptom of a cancer after the treatment. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the particle has a surface area of at least about 1 μm², or has a diameter of at least 0.5 μm. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual has minimal residual disease (MRD). In some embodiments, the individual has minimal residual cancer. In some embodiments, the minimal residual cancer is seen after the cancer was surgical resected or cured. In some embodiments, the individual has been previously treated for cancer and exhibits no pathological symptom of a cancer after the treatment. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the plurality of MHC-peptide complexes comprise a) at least two different MHC molecules, and b) at least two different peptides. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual has minimal residual disease (MRD). In some embodiments, the individual has minimal residual cancer. In some embodiments, the minimal residual cancer is seen after the cancer was surgical resected or cured. In some embodiments, the individual has been previously treated for cancer and exhibits no pathological symptom of a cancer after the treatment. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the display moiety comprises at least two different MHC class I molecules. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, there is provided a methods of analyzing immune cells in a sample from an individual, comprising a) contacting a plurality of immune cells with one or more display moieties, and b) detecting or analyzing the one or more immune cells that bind to one or more of the display moieties, wherein the immune cells are obtained from a sample (e.g., PBMCs) of an individual, wherein the individual has minimal residual disease (MRD). In some embodiments, the individual has minimal residual cancer. In some embodiments, the minimal residual cancer is seen after the cancer was surgical resected or cured. In some embodiments, the individual has been previously treated for cancer and exhibits no pathological symptom of a cancer after the treatment. In some embodiments, the number of cells in the sample (e.g., PBMCs) is no more than about 5×10⁶, 2×10⁶, 10⁶, 5×10⁵, 2×10⁵, 10⁵, 5×10⁴, 4×10⁴, 2×10⁴, or 10⁴ cells. In some embodiments, each of the one or more display moieties comprises a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the display moiety comprises at least two different MHC class II molecules. In some embodiments, the one or more display moieties are comprised in a bait composition. In some embodiments, the bait composition comprises a) at least two different (or at least three different) different MHC I molecules, and b) at least about 20, 50, 100, 150, 200, or 250 different peptides. In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01. In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule. In some embodiments, the peptides in the bait composition are associated with at least 25, 50, 75, 100, 125, 150, 175, 200, 225, or 250 different antigens. In some embodiments, the bait composition comprises at least 2, 3, 4, 5, 6, or 7 different overlapping peptides associated a mutation in a gene. In some embodiments, the gene is an oncogene. In some embodiments, the display moieties or the bait composition does not comprise a fluorochrome or fluorophore. In some embodiments, In some embodiments, the MHC molecules match with at least one, two, three, four, five or six HLA type of the individual from where the sample is obtained. In some embodiments, the immune cells are T cells (e.g., CD8+ T cells or CD4+ T cells). In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, the immune cells are immobilized to a solid substrate, e.g., via an agent binding to the immune cells that is coated to or conjugated with the solid substrate. In some embodiments, the agent is an antibody that specifically recognizes a surface marker of the immune cells. In some embodiments, the agent is an anti-CD8 antibody and the immune cells are CD8 T cells. In some embodiments, the agent is an anti-CD4 antibody and the immune cells are CD4 T cells. In some embodiments, the agent is an anti-CD3 antibody and the immune cells are T cells. The immobilization can be carried out by mixing immune cells with the agent (or incubating the immune cells with the agent) that specifically binds to the immune cells. In some embodiments, the agent is already coated or conjugated on a solid substrate, such as a plate or magnetic bead. The immune cells are allowed to be contacting with the agent for a period time and then a washing step can be applied to remove the cells not bound to the agent (e.g., using a magnet). See e.g., Examples.

In some embodiments, the number of immune cells is no more than about 10⁸, 5×10⁷, 10⁷, 5×10⁶, 2×10⁶, 10⁶, 5×10⁵, 2×10⁵, 10⁵, 5×10⁴, 4×10⁴, 2×10⁴, or 10⁴ cells.

In some embodiments, the solid substrate comprises a plate or a surface of a particle (such as a surface of a particle which is different from the particle used as support in display moieties, such as an inner surface of a porous particle). In some embodiments, the solid substrate comprises any of beads (e.g., magnetic beads), porous beads, porous matrices, arrays, glass surfaces, silicon surfaces, plastic surfaces, filters, membranes, nylons, silicon wafer chips, flow-through chips. In some embodiments, the solid substrate comprises any of a biochip containing electronics, a microtiter well, an ELISA plate, a spin interference disc, a nitrocellulose membrane, a nitrocellulose-based polymer surface, a nanoparticle, or a microsphere.

In some embodiments, contacting a plurality of immune cells with the display moiety or a bait composition is conducted under a condition sufficient for an immune cell to bind to the display moieties. In some embodiments, the “condition” in the term “under a condition sufficient for an immune cell to bind to the display moieties” refers to conditions in which specific binding between the immune cells and the display moieties happen. In some embodiments, it refers to conditions that involve about or at least about 5, 10, 15, 20, 25, or 30 minutes of incubation of the immune cells and display moieties (e.g., at room temperature in a medium). In some embodiments, it refers to conditions under at least 10%, 20%, 30%, 40%, 50%, 60%, 70%, 80%, 90% of the maximum binding between the immune cells and the display moieties happen. Maximum binding can be assessed by, e.g., contacting immune cells with display moieties for adequate time (e.g., 30 minutes) under a proper condition (e.g., room temperature).

In some embodiments, the sample is a mixture a plurality of individual samples. In some embodiments, the plurality of individual samples are from a single individual. In some embodiments, the plurality of individual samples are from a plurality of individuals.

In some embodiments, the sample is a mixture a plurality of individual samples and each of the plurality of individual samples is obtained from a different individual. In some embodiments, the method comprises prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. The association can be achieved by e.g., mixing immune cells with a molecule (e.g., an antibody that binds to the immune cells, e.g., a CD45 antibody) that comprises the unique barcode and binds to the immune cells for them to bind to each other. For example, unique barcode can be associated to cells via e.g., an antibody conjugated or fused with the barcode that specifically binds to the cells. In some embodiments, the barcode is a nucleic acid barcode (e.g., DNA barcode). In some embodiments, the unique DNA barcode is randomized barcode with a length of about 10-20 nucleic acids (e.g., 12-18 nucleic acids). In some embodiments, the barcode is a peptide barcode. In some embodiments, the method further comprises removing unbound molecule comprising the barcode (molecules not bound by the immune cells) e.g., via washing the immune cells for at least 1, 2, or 3 times.

In some embodiments, the method further comprises determining the unique barcode associated with the immune cells bound to the display moieties, thereby identifying the sample from which the immune cells are derived. For example, nucleic acids can be purified, and sequencing can used to identify the immune cells with which a unique DNA barcode is associated with.

In some embodiments, the individual is a human. In some embodiments, the individual is at least about 50 years old (e.g., at least 50, 60, 70, or 80 years old).

In some embodiments, the sample described herein (including this section and any other sections of the present application) can be any sample from the individual. In some embodiments, the sample described herein is a blood sample. In some embodiments, the sample described herein is a plasma sample. In some embodiments, the sample described herein comprises peripheral blood mononuclear cell (PMBC). In some embodiments, the number of PBMCs is no more than about 10⁸, 5×10⁷, 10⁷, 5×10⁶, 2×10⁶, 10⁶, 5×10⁵, 2×10⁵, 10⁵, 5×10⁴, 4×10⁴, 2×10⁴, or 10⁴ cells. In some embodiments, the sample described herein is a sample (e.g., a biopsy sample) obtained from a tissue or organ of the individual. In some embodiments, the sample is obtained from a lymph node of the individual.

The methods described herein have high specificity and sensitivity that enable it to detect antigen-specific immune cells even when they are rare in the sample (e.g., less than 1%). In some embodiments, the sample comprises PBMC and the immune cells comprise or are T cells or B cells contained in the PBMC. In some embodiments, the immune cells are not subject to an enrichment step for the immune cells in general or immune cells that specifically binds to the peptide prior to contacting with the bait composition. In some embodiments, the immune cells are not subject to an enrichment step for the immune cells in general or immune cells that specifically binds to the peptide throughout this methods. In some embodiments, the sample comprise no more than 1×10⁸, 5×10⁷, 2×10⁷, 1×10⁷, 5×10⁶, 2×10⁶, or 1×10⁶ PBMCs. In some embodiments, the sample comprise no more than 1×10⁶, 5×10⁵, 2×10⁵, 1×10⁵, 5×10⁴, 2×10⁴, 1×10⁴, 7×10³, 5×10³, 2×10³, 1×10³, 5×10², 2×10², or 1×10² immune cells (e.g., CD8 T cells, e.g., immune cells that bind to peptide(s) in the bait composition, e.g., CD4 or CD8 T cells that bind to peptide(s) in the bait composition).

### Methods of Detecting Antigen-Specific Immune Cells in an Individual

In some embodiments, there is provided a method of detecting antigen-specific immune cells in an individual (e.g., an individual not exhibiting a pathological symptom of a cancer or tumor, an infection by a particular pathogen, or an autoimmune disease or condition associated with an autoantigen, e.g., an individual being at risk of developing the disease or condition, such as cancer, infection or autoimmune disease or disorder, e.g., an individual who has not been treated for the disease or condition, e.g., an individual who has been treated with disease or condition such as cancer and is suspected to have minimal residual cancer cells), comprising: analyzing a sample from the individual according to any of the methods described herein, wherein a predetermined characteristic of the immune cells is indicative of a disease or a disorder in the individual. In some embodiments, the predetermined characteristic of the immune cell comprises the presence of the immune cells. In some embodiments, the presence of immune cells specifically binds to a bait composition comprising one or more neoantigens is indicative of the presence of a cancer. In some embodiments, the presence of immune cells specifically binds to a bait composition comprising one or more antigens of a pathogen (e.g., virus, bacteria, fungus) is indicative of the presence of an infection of the pathogen. In some embodiments, the presence of immune cells specifically binds to a bait composition comprising one or more autoantigens associated with an autoimmune disease or disorder is indicative of the presence of the immune disease or disorder. In some embodiments, the predetermined characteristic of the immune cell comprises a quantity of the immune cell above a threshold level. In some embodiments, the predetermined characteristic of the immune cell comprises a gene expression profile signature, a gene mutation profile signature, and/or an epigenetic modification signature. In some embodiments, the signature epigenetic modification comprises a DNA methylation signature and a histone glycosylation signature.

In some embodiments, there is provided a method of detecting antigen-specific immune cells in an individual (e.g., an individual not exhibiting a pathological symptom of a cancer or tumor, an infection by a particular pathogen, or an autoimmune disease or condition associated with an autoantigen, e.g., an individual being at risk of developing the disease or condition, such as cancer, infection or autoimmune disease or disorder, e.g., an individual who has not been treated for the disease or condition, e.g., an individual who has been treated with disease or condition such as cancer and is suspected to have minimal residual cancer cells), comprising: a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety; b) contacting the immobilized immune cells with a detection agent recognizing the display moiety under a condition sufficient for the agent to bind to the display moiety; and c) detecting the detection agent, thereby detecting the immune cells bound to the display moiety. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time. In some embodiments, the immune cells have been contacted with a bait composition comprising a display moiety comprising an antigenic peptide under a condition sufficient for an immune cell to bind to the display moiety prior to the immobilization. In some embodiments, the immune cells are contacted with a bait composition comprising a display moiety comprising an antigenic peptide under a condition sufficient for an immune cell to bind to the display moiety following the immobilization.

In some embodiments, there is provided a method of detecting antigen-specific immune cells in an individual (e.g., an individual not exhibiting a pathological symptom of a cancer or tumor, an infection by a particular pathogen, or an autoimmune disease or condition associated with an autoantigen, e.g., an individual being at risk of developing the disease or condition, such as cancer, infection or autoimmune disease or disorder, e.g., an individual who has not been treated for the disease or condition, e.g., an individual who has been treated with disease or condition such as cancer and is suspected to have minimal residual cancer cells), comprising: a) contacting the immune cells with a bait composition comprising a display moiety comprising the antigenic peptide under a condition sufficient for an immune cell to bind to the display moiety; b) contacting the immune cells with a detection agent recognizing the display moiety under a condition sufficient for the agent to bind to the display moiety; c) immobilizing the immune cells from the sample on a solid support, and d) detecting the detection agent, thereby detecting the immune cells bound to the display moiety. In some embodiments, the sample is obtained from a single individual. In some embodiments, the sample is a mixture a plurality of individual samples, each obtained from a different individual. In some embodiments, prior to being combined to form the mixture each of the plurality of individual samples is separately processed such that the immune cells contained therein are associated with a unique sample barcode.

In some embodiments, there is provided a method of detecting antigen-specific immune cells in a plurality of individuals (e.g., a plurality of individuals a) not exhibiting a pathological symptom of a cancer or tumor, an infection by a particular pathogen, or an autoimmune disease or condition associated with an autoantigen, b) being at risk of developing the disease or condition, such as cancer, infection or autoimmune disease or disorder, c) who has not been treated for the disease or condition, or d) who has been treated with disease or condition such as cancer and is suspected to have minimal residual cancer cells), comprising: a) separately processing each of the plurality of individual samples such that immune cells contained therein are associated with a unique sample barcode, b) combining the plurality of processed samples to form a sample mixture, c) contacting the sample mixture with a bait composition comprising a display moiety comprising an antigenic peptide under a condition sufficient for an immune cell to bind to the display moiety, thereby forming a bait-sample mixture, d) detecting immune cells bound to the display moiety in the bait-sample mixture; and e) determining the unique barcode associated with the immune cells bound to the display moiety, thereby identifying the sample from which the immune cells are derived.

In some embodiments, there is provided a method of detecting antigen-specific immune cells in an individual (e.g., an individual not exhibiting a pathological symptom of a cancer or tumor, an infection by a particular pathogen, or an autoimmune disease or condition associated with an autoantigen, e.g., an individual being at risk of developing the disease or condition, such as cancer, infection or autoimmune disease or disorder, e.g., an individual who has not been treated for the disease or condition, e.g., an individual who has been treated with disease or condition such as cancer and is suspected to have minimal residual cancer cells), comprising: a) contacting the sample with a bait composition comprising an display moiety comprising an antigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the immune cell, wherein a predetermined characteristic of the immune cell is indicative of disease or condition in the individual, and wherein the predetermined characteristic of the immune cell comprises the presence of the immune cell, optionally wherein the predetermined characteristic of the immune cell comprises a quantity of the immune cell above a threshold level. In some embodiments, the predetermined characteristic of the immune cell further comprises a gene expression profile signature, a gene mutation profile signature, and/or an epigenetic modification signature (e.g., a DNA methylation signature and a histone glycosylation signature). In some embodiments, the display moiety comprises two or more (e.g., four) antigenic peptides. In some embodiments, the two or more antigenic peptides in the display moiety are the same. In some embodiments, the antigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the antigenic peptide has low immunogenicity. In some embodiments, the display moiety comprises an MHC molecule complexed with the antigenic peptide. In some embodiments, the MHC molecule is a MHC class I molecule and/or a MHC class II molecule. In some embodiments, the immune cell is an isolated single immune cell. In some embodiments, the immune cell is in a mixture of immune cells. In some embodiments, the mixture of immune cells is a mixture comprising T cells, memory T cells, macrophage cells, or dendritic cells, or combinations thereof. In some embodiments, further analyzing the immune cell comprises quantifying the immune cell. In some embodiments, further analyzing the immune cell comprises sequencing one or more nucleic acids in the immune cell, optionally further comprising analyzing the sequences of the one or more nucleic acids (e.g., a TCR related sequence). In some embodiments, further analyzing the immune cell further comprises identifying one or more epigenetic modifications (e.g., DNA or RNA methylation, hydroxymethylation, and/or histone modifications such as acetylation, methylation, glycosylation) in the immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different antigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the immune cell to mass spectrometry analysis.

In some embodiments, there is provided a method of detecting antigen-specific immune cells in an individual (e.g., an individual not exhibiting a pathological symptom of a cancer or tumor, an infection by a particular pathogen, or an autoimmune disease or condition associated with an autoantigen, e.g., an individual being at risk of developing the disease or condition, such as cancer, infection or autoimmune disease or disorder, e.g., an individual who has not been treated for the disease or condition, e.g., an individual who has been treated with disease or condition such as cancer and is suspected to have minimal residual cancer cells), comprising: a) contacting the sample with a bait composition comprising an display moiety comprising an antigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the immune cell, wherein a predetermined characteristic of the immune cell is indicative of cancer in the individual, and wherein the predetermined characteristic of the immune cell comprise a gene expression profile signature. In some embodiments, the predetermined characteristic of the immune cell further comprises a gene mutation profile signature, and/or an epigenetic modification signature (e.g., a DNA methylation signature and a histone glycosylation signature). In some embodiments, the display moiety comprises two or more (e.g., four) antigenic peptides. In some embodiments, the two or more antigenic peptides in the display moiety are the same. In some embodiments, the antigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the antigenic peptide has low immunogenicity. In some embodiments, the display moiety comprises an MHC molecule complexed with the antigenic peptide. In some embodiments, the MHC molecule is a MHC class I molecule and/or a MHC class II molecule. In some embodiments, the immune cell is an isolated single immune cell. In some embodiments, the immune cell is in a mixture of immune cells. In some embodiments, the mixture of immune cells is a mixture comprising T cells, memory T cells, macrophage cells, or dendritic cells, or combinations thereof. In some embodiments, further analyzing the immune cell comprises detecting and/or quantifying the immune cell. In some embodiments, further analyzing the immune cell comprises sequencing one or more nucleic acids in the immune cell, optionally further comprising analyzing the sequences of the one or more nucleic acids (e.g., a TCR related sequence). In some embodiments, further analyzing the immune cell further comprises identifying one or more epigenetic modifications (e.g., DNA or RNA methylation, hydroxymethylation, and/or histone modifications such as acetylation, methylation, glycosylation) in the immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different antigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the immune cell to mass spectrometry analysis.

In some embodiments, there is provided a method of detecting cancer in an individual (e.g., an individual who has not been diagnosed as having a cancer, e.g., an individual being at risk of developing a cancer, e.g., an individual who have been treated for cancer), comprising: a) contacting the sample with a bait composition comprising an display moiety comprising a cancer antigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the immune cell, wherein a predetermined characteristic of the immune cell is indicative of cancer in the individual, and wherein the predetermined characteristic of the immune cell comprises a gene mutation profile signature, and/or an epigenetic modification signature (e.g., a DNA methylation signature and a histone glycosylation signature). In some embodiments, the display moiety comprises two or more (e.g., four) antigenic peptides. In some embodiments, the two or more antigenic peptides in the display moiety are the same. In some embodiments, the antigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the antigenic peptide has low immunogenicity. In some embodiments, the display moiety comprises an MHC molecule complexed with the antigenic peptide. In some embodiments, the MHC molecule is a MHC class I molecule and/or a MHC class II molecule. In some embodiments, the immune cell is an isolated single immune cell. In some embodiments, the immune cell is in a mixture of immune cells. In some embodiments, the mixture of immune cells is a mixture comprising T cells, memory T cells, macrophage cells, or dendritic cells, or combinations thereof. In some embodiments, further analyzing the immune cell comprises detecting and/or quantifying the immune cell. In some embodiments, further analyzing the immune cell comprises sequencing one or more nucleic acids in the immune cell, optionally further comprising analyzing the sequences of the one or more nucleic acids (e.g., a TCR related sequence). In some embodiments, further analyzing the immune cell further comprises identifying one or more epigenetic modifications (e.g., DNA or RNA methylation, hydroxymethylation, and/or histone modifications such as acetylation, methylation, glycosylation) in the immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different antigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the immune cell to mass spectrometry analysis.

In some embodiments, there is provided a method of detecting residual cancer in an individual, wherein the individual has been previously treated with an anti-cancer therapy and exhibits no pathological symptom of cancer after treatment, the method comprising analyzing a post-treatment sample from the individual. In some embodiments, the method further comprises a) analyzing a pre-treatment sample from the individual prior to anti-cancer therapy and a post-treatment sample from the individual according to any of the methods described herein, and b) comparing the characteristics of the immune cells from the pre-treatment sample and isolated immune cells from the post-treatment sample. In some embodiments, a predetermined difference in characteristics of the immune cell from the pre-treatment sample and the immune cell from the post-treatment sample is indicative of residual cancer in the individual. In some embodiments, the predetermined characteristic of the immune cell comprises the presence of the immune cell, optionally further comprising a quantity of the immune cell above a threshold level. In some embodiments, the predetermined characteristic of the immune cell comprises a gene expression profile signature, a gene mutation profile signature, and/or an epigenetic modification signature (e.g., a DNA methylation signature and/or a histone glycosylation signature). In some embodiments, the method comprises: a) contacting the pre-treatment and/or post-treatment sample with a bait composition comprising an display moiety comprising a cancer antigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the immune cell. In some embodiments, the display moiety comprises two or more (e.g., four) antigenic peptides. In some embodiments, the two or more antigenic peptides in the display moiety are the same. In some embodiments, the antigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the antigenic peptide has low immunogenicity. In some embodiments, the display moiety comprises an MHC molecule complexed with the antigenic peptide. In some embodiments, the MHC molecule is a MHC class I molecule and/or a MHC class II molecule. In some embodiments, the immune cell is an isolated single immune cell. In some embodiments, the immune cell is in a mixture of immune cells. In some embodiments, the mixture of immune cells is a mixture comprising T cells, memory T cells, macrophage cells, or dendritic cells, or combinations thereof. In some embodiments, further analyzing the immune cell comprises detecting and/or quantifying the immune cell. In some embodiments, further analyzing the immune cell comprises sequencing one or more nucleic acids in the immune cell, optionally further comprising analyzing the sequences of the one or more nucleic acids (e.g., a TCR related sequence). In some embodiments, further analyzing the immune cell further comprises identifying one or more epigenetic modifications (e.g., DNA or RNA methylation, hydroxymethylation, and/or histone modifications such as acetylation, methylation, glycosylation) in the immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different antigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the immune cell to mass spectrometry analysis.

### Agglutination Test

In some embodiments, the methods described herein (such as methods of analyzing immune cells or detecting antigen-specific immune cells) involve (e.g., comprises) conducting an agglutination test. In some embodiments, the agglutination test comprises observing the agglutination of cells under a microscope or bare eyes. In some embodiments, the number of immune cells is no more than about 10⁸, 5×10⁷, 10⁷, 5×10⁶, 2×10⁶, 10⁶, 5×10⁵, 2×10⁵, 10⁵, 5×10⁴, 4×10⁴, 2×10⁴, or 10⁴ cells.

Agglutination tests are a class of bioanalytical methods used to detect the presence (qualitive) and sometimes the amount of a substance (quantitative) in a sample. The process generally involves the clumping or aggregation of particles in response to a specific substance, such as an antigen, antibody, or cell. In some embodiments, the agglutination reaction occurs with specially designed particles and is often utilized in various diagnostic applications, including blood typing, pathogen detection, and measuring antibody levels.

In some embodiments, in an agglutination test, antibodies or other binding agents are mixed with a sample containing potential target antigens. If the antigens are present, they bind to the antibodies, causing the particles to clump together, or agglutinate. This clumping can be observed directly or quantified using various detection methods. In some embodiments, the reaction is controlled to avoid non-specific agglutination, which can lead to false-positive results. In some embodiments, various buffers and additives may be used to achieve the desired reaction conditions, including pH, ionic strength, and temperature. In some embodiments, the test may be performed in various formats, such as slide agglutination, tube agglutination, or microplate agglutination, depending on the sample type, target concentration, and required throughput.

In some embodiments, a latex beads-based agglutination test (LBAT) is utilized to employ latex beads as the carrier particles for the binding agents. In some embodiments, these latex beads offer several advantages, including uniform size, shape, and surface properties, which can be precisely controlled to optimize the test's performance. In some embodiments, the latex beads can be functionalized with various chemical groups and conjugated to appropriate reagents such as antibodies or antigens, or they can passively bind to these reagents.

In some embodiments, the LBAT comprises bead functionalization. The latex beads are functionalized with the required binding agents. This may involve chemical activation, coupling chemistry, and purification steps. In some embodiments, the LBAT comprises sample preparation. The sample is prepared according to the specific requirements of the test, which may include dilution, filtration, or other preprocessing steps. In some embodiments, the LBAT comprises reaction mixing: The functionalized beads are mixed with the sample in a controlled environment. Buffer solutions, stabilizers, or other additives may be used to ensure optimal reaction conditions. In some embodiments, the LBAT comprises incubation. The reaction mixture is incubated to allow time for the agglutination to occur. The temperature, time, and agitation may be adjusted to achieve the desired reaction kinetics. In some embodiments, the LBAT comprises detection. The agglutination is observed or quantified using appropriate detection methods. This may include visual inspection, microscopy, or instrumental analysis, such as spectrophotometry.

In some embodiments, the use of latex beads in agglutination tests allows for the customization of the beads to meet the unique requirements of different applications, including infectious disease diagnosis, immunological research, food testing, etc.

Latex beads, colloquial particles developed in the mid-20th century, play a significant role in advancing diagnostics and molecular biology. In some embodiments, these beads are synthesized through emulsion polymerization, where monomers are polymerized in an aqueous phase to form polymer beads. In some embodiments, this process often involves the use of stabilizers and surfactants to control the size and shape of the particles. In some embodiments, subsequent refinements in synthesis techniques have allowed for greater uniformity and control over the physical properties of the particles, such as size, shape, and surface charge.

In some embodiments, Latex beads have a core made of synthetic polymers, with polystyrene being the most common core material. In some embodiments, it is best known for its structural strength and for being chemically and physically inert when interacting with biological systems. In some embodiments, other variants of latex beads have been synthesized using polymers such as poly(methyl methacrylate) (PMMA) and poly(lactic-co-glycolic acid) (PLGA), a biodegradable polymer known for its use in drug delivery applications. In some embodiments, the size of latex beads can be finely controlled and typically ranges from about 20 nanometers to about 100 micrometers in diameter. In some embodiments, the core of the beads often incorporates a crosslinking agent, such as divinylbenzene (DVB). In some embodiments, crosslinking imparts a three-dimensional network within the polymer, greatly enhancing the structural integrity and solvent resistance of the beads.

In some embodiments, the latex beads have a diameter of about at least 0.01, 0.05, 0.1, 0.2, 0.3, 0.4, 0.5, 0.6, 0.7, 0.8, 0.9, or 1 μm. In some embodiments, the latex beads have a diameter of about 0.01 to about 150 μm, or 0.1 to about 100 μm (such as about 0.1 to about 50 μm, such as about 0.5 μm to about 20 μm, such as about 0.5 μm to about 10 μm, such as about 0.5 μm to about 5 μm, such as about 0.5 μm to about 2.5 μm). In some embodiments, the latex beads have a diameter of about 0.1 to about 10 μm. In some embodiments, the latex beads have a diameter of about 0.5 to about 2 μm. In some embodiments, the latex beads have a diameter of about 1 μm.

In some embodiments, the surface modification of latex beads is central to their utility in various applications, as it allows them to be tailored to specific test requirements. In some embodiments, the beads can be designed to conjugate various biological targets such as antibodies, proteins, carbohydrates, DNA, or RNA by introducing different surface functional groups. In some embodiments, carboxylated beads, for instance, provide a reactive surface for covalent conjugation of biomolecules, making them ideal for immobilizing proteins or antibodies through carbodiimide chemistry. In some embodiments, amine-functionalized beads contain primary amine groups that can react with various target molecules, and are commonly utilized for oligonucleotide and peptide conjugation. In some embodiments, aldehyde-functionalized beads have the capacity to react with amine groups to form Schiff bases, making them suitable for protein coupling, while hydroxyl beads can be used for additional modifications or as blank controls. In some embodiments, specialized functionalization options include magnetic beads, fluorescently-labeled beads, and biotinylated beads that can bind to streptavidin or avidin, enhancing the flexibility and adaptability of latex beads across a broad range of applications.

### Methods of Treatment

In some embodiments, there is provided a method of treating a cancer in an individual (e.g., an individual who has not been diagnosed as having a cancer, e.g., an individual being at risk of developing a cancer, e.g., an individual who have been treated for cancer), comprising a) diagnosing the individual as having cancer according to the methods described herein; and b) subjecting the individual to an anti-cancer therapy. In some embodiments, diagnosing the individual comprises a) obtaining a sample from the individual, b) contacting the sample with a bait composition comprising an display moiety comprising a cancer antigenic peptide under a condition sufficient for an immune cell (e.g., a T cell, a cytotoxic T cell, a helper T cell, a memory T cell, and/or a tumor infiltrating T cell) to bind to the display moiety; c) isolating an immune cell associated with the display moiety; and d) analyzing the immune cell, wherein a predetermined characteristic of the immune cell is indicative of cancer in the individual. In some embodiments, the predetermined characteristic of the immune cell comprises the presence of the immune cell. In some embodiments, the predetermined characteristic of the immune cell comprises a quantity of the immune cell above a threshold level. In some embodiments, the predetermined characteristic of the immune cell comprises a gene expression profile signature, a gene mutation profile signature, and/or an epigenetic modification signature (e.g., a DNA methylation signature and/or a histone glycosylation signature). In some embodiments, the display moiety comprises two or more (e.g., four) antigenic peptides. In some embodiments, the two or more antigenic peptides in the display moiety are the same. In some embodiments, the antigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the antigenic peptide has low immunogenicity. In some embodiments, the display moiety comprises an MHC molecule complexed with the antigenic peptide. In some embodiments, the MHC molecule is a MHC class I molecule and/or a MHC class II molecule. In some embodiments, the immune cell is an isolated single immune cell. In some embodiments, the immune cell is in a mixture of immune cells. In some embodiments, the mixture of immune cells is a mixture comprising T cells, memory T cells, macrophage cells, or dendritic cells, or combinations thereof. In some embodiments, further analyzing the immune cell comprises detecting and/or quantifying the immune cell. In some embodiments, further analyzing the immune cell comprises sequencing one or more nucleic acids in the immune cell, optionally further comprising analyzing the sequences of the one or more nucleic acids (e.g., a TCR related sequence). In some embodiments, further analyzing the immune cell further comprises identifying one or more epigenetic modifications (e.g., DNA or RNA methylation, hydroxymethylation, and/or histone modifications such as acetylation, methylation, glycosylation) in the immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different antigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the immune cell to mass spectrometry analysis.

In some embodiments, the anti-cancer therapy is a standard or commonly used agent or therapy for treating cancer (e.g., a specific cancer). In some embodiments, the anti-cancer therapy comprises a chemotherapeutic agent. In some embodiments, the anti-cancer therapy comprises a surgery. In some embodiments, the anti-cancer therapy comprises a radiation therapy. In some embodiments, the anti-cancer therapy comprises an immunotherapy. In some embodiments, the anti-cancer therapy comprises a cell therapy (such as a cell therapy comprising an immune cell (e.g., CAR T cell)). In some embodiments, the anti-cancer therapy comprises an angiogenesis inhibitor.

In some embodiments, the anti-cancer therapy is not an immunotherapy.

In some embodiments, the individual has minimal residual disease (MRD). In some embodiments, the individual has minimal residual cancer. In some embodiments, the minimal residual cancer is seen after the cancer was surgical resected or cured. In some embodiments, the minimal residual disease is too minimal to be detected by imaging instruments (e.g., a routinely used or standard imaging instrument used for detection of the cancer). In some embodiments, the location of the minimal residual disease is diverse. In some embodiments, the minimal residual cancer is a result of immune escape or resistance to treatment. In some embodiments, the individual has been previously treated for cancer and exhibits no pathological symptom of a cancer after the treatment.

In some embodiments, there is provided a method of treating an infection in an individual (e.g., an individual who has not been diagnosed as having an infection by a pathogen such as a virus, bacteria or fungus, e.g., an individual being at risk of developing the infection, e.g., an individual who have been treated for infection), comprising a) diagnosing the individual as having infection according to the methods described herein; and b) subjecting the individual to an anti-infection therapy. In some embodiments, diagnosing the individual comprises a) obtaining a sample from the individual, b) contacting the sample with a bait composition comprising an display moiety comprising an infection antigenic peptide under a condition sufficient for an immune cell (e.g., a T cell, a cytotoxic T cell, a helper T cell, a memory T cell, and/or a tumor infiltrating T cell) to bind to the display moiety; c) isolating an immune cell associated with the display moiety; and d) analyzing the immune cell, wherein a predetermined characteristic of the immune cell is indicative of infection in the individual. In some embodiments, the predetermined characteristic of the immune cell comprises the presence of the immune cell. In some embodiments, the predetermined characteristic of the immune cell comprises a quantity of the immune cell above a threshold level. In some embodiments, the predetermined characteristic of the immune cell comprises a gene expression profile signature, a gene mutation profile signature, and/or an epigenetic modification signature (e.g., a DNA methylation signature and/or a histone glycosylation signature). In some embodiments, the display moiety comprises two or more (e.g., four) antigenic peptides. In some embodiments, the two or more antigenic peptides in the display moiety are the same. In some embodiments, the antigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the antigenic peptide has low immunogenicity. In some embodiments, the display moiety comprises an MHC molecule complexed with the antigenic peptide. In some embodiments, the MHC molecule is a MHC class I molecule and/or a MHC class II molecule. In some embodiments, the immune cell is an isolated single immune cell. In some embodiments, the immune cell is in a mixture of immune cells. In some embodiments, the mixture of immune cells is a mixture comprising T cells, memory T cells, macrophage cells, or dendritic cells, or combinations thereof. In some embodiments, further analyzing the immune cell comprises detecting and/or quantifying the immune cell. In some embodiments, further analyzing the immune cell comprises sequencing one or more nucleic acids in the immune cell, optionally further comprising analyzing the sequences of the one or more nucleic acids (e.g., a TCR related sequence). In some embodiments, further analyzing the immune cell further comprises identifying one or more epigenetic modifications (e.g., DNA or RNA methylation, hydroxymethylation, and/or histone modifications such as acetylation, methylation, glycosylation) in the immune cell. In some embodiments, the method further comprises generating a report comprising information about the infection status in the individual. In some embodiments, the information about infection status comprises: classification of infection; type of infection; nature of infection; origin of infection; stage of infection; likelihood of infection progression; likelihood of developing one or more infection symptoms; molecular diagnosis; NGS pathology; and/or treatment options for the individual. In some embodiments, the bait composition comprises a plurality of different display moieties. In some embodiments, each of the plurality of different display moieties in the bait composition comprises a different antigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the immune cell to mass spectrometry analysis.

In some embodiments, there is provided a method of treating an autoimmune disease or condition in an individual (e.g., an individual who has not been diagnosed as having an autoimmune disease or condition, e.g., an individual being at risk of developing an autoimmune disease or condition, e.g., an individual who have been treated for autoimmune disease or condition), comprising a) diagnosing the individual as having autoimmune disease or condition according to the methods described herein; and b) subjecting the individual to an anti-autoimmune disease or condition therapy. In some embodiments, diagnosing the individual comprises a) obtaining a sample from the individual, b) contacting the sample with a bait composition comprising an display moiety comprising an autoimmune disease or condition antigenic peptide under a condition sufficient for an immune cell (e.g., a T cell, a cytotoxic T cell, a helper T cell, a memory T cell, and/or a tumor infiltrating T cell) to bind to the display moiety; c) isolating an immune cell associated with the display moiety; and d) analyzing the immune cell, wherein a predetermined characteristic of the immune cell is indicative of autoimmune disease or condition in the individual. In some embodiments, the predetermined characteristic of the immune cell comprises the presence of the immune cell. In some embodiments, the predetermined characteristic of the immune cell comprises a quantity of the immune cell above a threshold level. In some embodiments, the predetermined characteristic of the immune cell comprises a gene expression profile signature, a gene mutation profile signature, and/or an epigenetic modification signature (e.g., a DNA methylation signature and/or a histone glycosylation signature). In some embodiments, the display moiety comprises two or more (e.g., four) antigenic peptides. In some embodiments, the two or more antigenic peptides in the display moiety are the same. In some embodiments, the antigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the antigenic peptide has low immunogenicity. In some embodiments, the display moiety comprises an MHC molecule complexed with the antigenic peptide. In some embodiments, the MHC molecule is a MHC class I molecule and/or a MHC class II molecule. In some embodiments, the immune cell is an isolated single immune cell. In some embodiments, the immune cell is in a mixture of immune cells. In some embodiments, the mixture of immune cells is a mixture comprising T cells, memory T cells, macrophage cells, or dendritic cells, or combinations thereof. In some embodiments, further analyzing the immune cell comprises detecting and/or quantifying the immune cell. In some embodiments, further analyzing the immune cell comprises sequencing one or more nucleic acids in the immune cell, optionally further comprising analyzing the sequences of the one or more nucleic acids (e.g., a TCR related sequence). In some embodiments, further analyzing the immune cell further comprises identifying one or more epigenetic modifications (e.g., DNA or RNA methylation, hydroxymethylation, and/or histone modifications such as acetylation, methylation, glycosylation) in the immune cell. In some embodiments, the method further comprises generating a report comprising information about the autoimmune disease or condition status in the individual. In some embodiments, the information about autoimmune disease or condition status comprises: classification of autoimmune disease or condition; type of autoimmune disease or condition; nature of autoimmune disease or condition; origin of autoimmune disease or condition; stage of autoimmune disease or condition; likelihood of autoimmune disease or condition progression; likelihood of developing one or more autoimmune disease or condition symptoms; molecular diagnosis; NGS pathology; and/or treatment options for the individual. In some embodiments, the bait composition comprises a plurality of different display moieties. In some embodiments, each of the plurality of different display moieties in the bait composition comprises a different antigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the immune cell to mass spectrometry analysis.

### Bait Compositions, Display Moieties, MHC-Peptide Complexes, and Antigenic Peptides

In some embodiments, the present application provides a composition comprising one or more display moieties or bait compositions. Bait compositions described herein comprise one or more display moieties comprising one or more antigenic peptides (e.g., comprising one or more MHC-peptide complexes), wherein an immune cell (such as a T cell) binds (e.g., cognately binds) to the display moiety and/or antigenic peptide. Any of these bait compositions, display moieties can be used in the methods of analyzing a sample, methods of detecting antigen-specific immune cells and methods of treatment described herein.

### Display Moiety (i.e., MHC Multimer Unit)

In some embodiments, the display moiety described herein comprises an MHC molecule complexed with an antigenic peptide (e.g. a neoantigenic peptide, e.g., a truncal neoantigenic peptide).

The present application in one aspect provides display moieties that comprise a support (e.g., a particle) associated with a plurality of MHC-peptide complexes (e.g., MHC-peptide monomers), wherein at least two of the MHC-peptide complexes are different.

In some embodiments, the display moiety comprises a support (such as a particle) associated with a plurality of MHC-peptide complexes, wherein the plurality of MHC-peptide complexes comprises no less than or more than 2, 3, 4, 5, 6, 8, 9, 10, or 12 MHC-peptide complexes, wherein each of the MHC-peptide complex comprises at least one MHC molecule complexed with a peptide, wherein at least two or more of the MHC-peptide complexes are different.

In some embodiments, the display moiety does not comprise a fluorochrome or fluorophore.

In some embodiments, the present application provides a display moiety comprising a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex is an MHC-peptide monomer comprising an MHC molecule complexed with a peptide, wherein at least two MHC-peptide complexes are different. In some embodiments, the plurality of MHC-peptide complexes comprise at least 20 MHC-peptide complexes. In some embodiments, the plurality of MHC-peptide complexes 20, 50, 100, 500, 1,000, 5,000, 10,000, 20,000, 50,000, 10,0000, 15,000, 20,0000 MHC-peptide complexes. In some embodiments, the MHC-peptide complex is attached to the particle via a binding pair comprising a first binding component attached to MHC-peptide complex or a unit thereof (i.e., a MHC-peptide monomer) and a second binding component bound to the particle. In some embodiments, the first binding component comprises a biotin, and the second binding component comprises streptavidin. In some embodiments, the particle has a diameter of at least 0.5 μm (e.g., at least 1 μm). In some embodiments, the particle has a diameter of about 0.01 μm to about 100 μm (e.g., 0.5 μm to about 100 μm, e.g., 0.5 μm to about 20 μm). In some embodiments, the particle has a diameter of about 0.5 μm to about 10 μm. In some embodiments, the particle has a diameter of about 1 μm. In some embodiments, the particle has a surface area of about 0.01 μm² to about 1000 μm² (e.g., about 0.1 μm² to about 100 μm², about 1 μm² to about 20 μm²). In some embodiments, the particle has a number of about 10 to about 200,000 (e.g., about 100 to about 20000, e.g., about 10 to about 10000) second binding component associated with it. In some embodiments, the display moiety comprises at least two different MHC class I molecules. In some embodiments, wherein the display moiety comprises at least two different MHC class II molecules. In some embodiments, wherein the display moiety comprises at least one MHC class I molecule and at least one MHC class II molecule. In some embodiments, the display moiety comprises an HLA-A molecule. In some embodiments. In some embodiments, the HLA-A molecule comprises a mutation that reduces its binding to CD8. In some embodiments, the HLA-A molecule comprises an HLA A2 heavy chain with a A245V mutation. In some embodiments, the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety comprises an MHC class II molecule selected from the group consisting of HLA-DP, HLA-DQ, and HLA-DR. In some embodiments, the peptide is associated with a cancer or tumor antigen. In some embodiments, the peptide is associated with a neoantigen. In some embodiments, the peptide is associated with an antigen of a pathogen. In some embodiments, the pathogen is a virus, bacteria of fungus. In some embodiments, the peptide is associated with an autoantigen. In some embodiments, the display moiety comprises at least two different peptides (e.g., at least 2, 5, 10, 20, 30, 40, 50, 100, 200, 500, or 1000 different peptides). In some embodiments, the at least two distinct peptides are associated with two different antigens. In some embodiments, the two distinct antigens comprise two distinct mutations of one or more genes (e.g., oncogenes) associated with a disease (e.g., a cancer). In some embodiments, the oncogene has a driven mutation. In some embodiments, the oncogene comprises one or more of KRAS, EGFR, POLE, TP53, SMAD, BRCA1, CDKN2A, TNN, MUC16, BRAF and ALK. In some embodiments, the oncogene comprises TP53, KRAS, MUC16, SMAD, CDKN2A and TNN. In some embodiments, the at least two different peptides overlap (e.g., overlap for at least 2, 3, 4, 5, or 6 amino acids). In some embodiments, the at least two different peptides do not overlap. In some embodiments, the display moiety does not comprise a fluorochrome or fluorophore.

In some embodiments, the present application provides a display moiety comprising a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the plurality of MHC-peptide complexes comprises at least 20, 50, 100, 500, 1,000, 5,000, 10,000, 20,000, 50,000, 10,0000, 15,000, 20,000, 50,000, 100,000, 200,000, 500,000, or 1,000,000 MHC-peptide complexes. In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer. In some embodiments, the MHC-peptide complex is attached to the particle via a binding pair comprising a first binding component attached to MHC-peptide complex or a unit thereof (i.e., a MHC-peptide monomer) and a second binding component bound to the particle. In some embodiments, the first binding component comprises a biotin, and the second binding component comprises streptavidin, or vice versa. In some embodiments, the particle has a diameter of at least 0.5 μm (e.g., at least 1 μm). In some embodiments, the particle has a diameter of about 0.01 μm to about 100 μm (e.g., 0.5 μm to about 100 μm, e.g., 0.5 μm to about 20 μm). In some embodiments, the particle has a diameter of about 0.5 μm to about 10 μm. In some embodiments, the particle has a diameter of about 1 μm. In some embodiments, the particle has a surface area of about 0.01 μm² to about 1000 μm² (e.g., about 0.1 μm² to about 100 μm², about 1 μm² to about 20 μm²). In some embodiments, the particle has a number of about 10 to about 200,000 (e.g., about 100 to about 20000, e.g., about 10 to about 10000) second binding component associated with it. In some embodiments, the display moiety comprises at least two different MHC class I molecules. In some embodiments, wherein the display moiety comprises at least two different MHC class II molecules. In some embodiments, wherein the display moiety comprises at least one MHC class I molecule and at least one MHC class II molecule. In some embodiments, the display moiety comprises an HLA-A molecule. In some embodiments, the HLA-A molecule comprises a mutation that reduces its binding to CD8. In some embodiments, the HLA-A molecule comprises an HLA A2 heavy chain with a A245V mutation. In some embodiments, the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety comprises an MHC class II molecule selected from the group consisting of HLA-DP, HLA-DQ, and HLA-DR. In some embodiments, the peptide is associated with a cancer or tumor antigen. In some embodiments, the peptide is associated with a neoantigen. In some embodiments, the peptide is associated with an antigen of a pathogen. In some embodiments, the pathogen is a virus, bacteria of fungus. In some embodiments, the peptide is associated with an autoantigen. In some embodiments, the display moiety comprises at least two different peptides (e.g., at least 2, 5, 10, 20, 30, 40, 50, 100, 200, 500, or 1000 different peptides). In some embodiments, the at least two distinct peptides are associated with two different antigens. In some embodiments, the two distinct antigens comprise two distinct mutations of one or more genes (e.g., oncogenes) associated with a disease (e.g., a cancer). In some embodiments, the oncogene has a driven mutation. In some embodiments, the oncogene comprises one or more of KRAS, EGFR, POLE, TP53, SMAD, BRCA1, CDKN2A, TNN, MUC16, BRAF and ALK. In some embodiments, the oncogene comprises TP53, KRAS, MUC16, SMAD, CDKN2A and TNN. In some embodiments, the at least two different peptides overlap. In some embodiments, the at least two different peptides do not overlap. In some embodiments, the display moiety does not comprise a fluorochrome or fluorophore.

In some embodiments, the present application provides a display moiety comprising a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the particle has a surface area of at least about 1 μm², or has a diameter of at least 0.5 μm. In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer. In some embodiments, the MHC-peptide complex is attached to the particle via a binding pair comprising a first binding component attached to MHC-peptide complex or a unit thereof (i.e., a MHC-peptide monomer) and a second binding component bound to the particle. In some embodiments, the first binding component comprises a biotin, and the second binding component comprises streptavidin. In some embodiments, the particle has a diameter of about 0.5 μm to about 100 μm (e.g., 0.5 μm to about 100 μm, e.g., 1 μm to about 20 μm). In some embodiments, the particle has a diameter of about 1 μm to about 10 μm. In some embodiments, the particle has a diameter of about 1 μm. In some embodiments, the particle has a surface area of about 1 μm² to about 1000 μm² (e.g., about 1 μm² to about 100 μm², about 1 μm² to about 20 μm²). In some embodiments, the particle has a number of about 10 to about 200,000 (e.g., about 100 to about 20000, e.g., about 10 to about 10000) second binding component associated with it. In some embodiments, the display moiety comprises at least two different MHC class I molecules. In some embodiments, wherein the display moiety comprises at least two different MHC class II molecules. In some embodiments, wherein the display moiety comprises at least one MHC class I molecule and at least one MHC class II molecule. In some embodiments, the display moiety comprises an HLA-A molecule. In some embodiments, the HLA-A molecule comprises a mutation that reduces its binding to CD8. In some embodiments, the HLA-A molecule comprises an HLA A2 heavy chain with a A245V mutation. In some embodiments, the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety comprises an MHC class II molecule selected from the group consisting of HLA-DP, HLA-DQ, and HLA-DR. In some embodiments, the peptide is associated with a cancer or tumor antigen. In some embodiments, the peptide is associated with a neoantigen. In some embodiments, the peptide is associated with an antigen of a pathogen. In some embodiments, the pathogen is a virus, bacteria of fungus. In some embodiments, the peptide is associated with an autoantigen. In some embodiments, the display moiety comprises at least two different peptides (e.g., at least 2, 5, 10, 20, 30, 40, 50, 100, 200, 500, or 1000 different peptides). In some embodiments, the at least two distinct peptides are associated with two different antigens. In some embodiments, the two distinct antigens comprise two distinct mutations of one or more genes (e.g., oncogenes) associated with a disease (e.g., a cancer). In some embodiments, the oncogene has a driven mutation. In some embodiments, the oncogene comprises one or more of KRAS, EGFR, POLE, TP53, SMAD, BRCA1, CDKN2A, TNN, MUC16, BRAF and ALK. In some embodiments, the oncogene comprises TP53, KRAS, MUC16, SMAD, CDKN2A and TNN. In some embodiments, the at least two different peptides overlap. In some embodiments, the at least two different peptides do not overlap. In some embodiments, the display moiety does not comprise a fluorochrome or fluorophore.

In some embodiments, the present application provides a display moiety comprising a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the plurality of MHC-peptide complexes comprise a) at least two different MHC molecules, and b) at least two different peptides. In some embodiments, one or more of the plurality of MHC-peptide complexes is an MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer. In some embodiments, the MHC-peptide complex is attached to the particle via a binding pair comprising a first binding component attached to MHC-peptide complex or a unit thereof (i.e., a MHC-peptide monomer) and a second binding component bound to the particle. In some embodiments, the first binding component comprises a biotin, and the second binding component comprises streptavidin. In some embodiments, the particle has a diameter of at least 0.5 μm (e.g., at least 1 μm). In some embodiments, the particle has a diameter of about 0.01 μm to about 100 μm (e.g., 0.5 μm to about 100 μm, e.g., 0.5 μm to about 20 μm). In some embodiments, the particle has a diameter of about 0.5 μm to about 10 μm. In some embodiments, the particle has a diameter of about 1 μm. In some embodiments, the particle has a surface area of about 0.01 μm² to about 1000 μm² (e.g., about 0.1 μm² to about 100 μm², about 1 μm² to about 20 μm²). In some embodiments, the particle has a number of about 10 to about 200,000 (e.g., about 100 to about 20000, e.g., about 10 to about 10000) second binding component associated with it. In some embodiments, the display moiety comprises at least two different MHC class I molecules. In some embodiments, wherein the display moiety comprises at least two different MHC class II molecules. In some embodiments, wherein the display moiety comprises at least one MHC class I molecule and at least one MHC class II molecule. In some embodiments, the display moiety comprises an HLA-A molecule. In some embodiments, the HLA-A molecule comprises a mutation that reduces its binding to CD8. In some embodiments, the HLA-A molecule comprises an HLA A2 heavy chain with a A245V mutation. In some embodiments, the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety comprises an MHC class II molecule selected from the group consisting of HLA-DP, HLA-DQ, and HLA-DR. In some embodiments, the peptide is associated with a cancer or tumor antigen. In some embodiments, the peptide is associated with a neoantigen. In some embodiments, the peptide is associated with an antigen of a pathogen. In some embodiments, the pathogen is a virus, bacteria of fungus. In some embodiments, the peptide is associated with an autoantigen. In some embodiments, the display moiety comprises at least two different peptides (e.g., at least 2, 5, 10, 20, 30, 40, 50, 100, 200, 500, or 1000 different peptides). In some embodiments, the at least two distinct peptides are associated with two different antigens. In some embodiments, the two distinct antigens comprise two distinct mutations of one or more genes (e.g., oncogenes) associated with a disease (e.g., a cancer). In some embodiments, the oncogene has a driven mutation. In some embodiments, the oncogene comprises one or more of KRAS, EGFR, POLE, TP53, SMAD, BRCA1, CDKN2A, TNN, MUC16, BRAF and ALK. In some embodiments, the oncogene comprises TP53, KRAS, MUC16, SMAD, CDKN2A and TNN. In some embodiments, the at least two different peptides overlap. In some embodiments, the at least two different peptides do not overlap. In some embodiments, the display moiety does not comprise a fluorochrome or fluorophore.

In some embodiments, the present application provides a display moiety comprising a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the display moiety comprises at least two different MHC class I molecules. In some embodiments, at least one or each of the plurality of MHC-peptide complexes is an MHC-peptide monomer, MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer. In some embodiments, the MHC-peptide complex is attached to the particle via a binding pair comprising a first binding component attached to MHC-peptide complex or a unit thereof (i.e., a MHC-peptide monomer) and a second binding component bound to the particle. In some embodiments, the first binding component comprises a biotin, and the second binding component comprises streptavidin. In some embodiments, the particle has a diameter of about 0.5 μm to about 100 μm (e.g., 0.5 μm to about 100 μm, e.g., 1 μm to about 20 μm). In some embodiments, the particle has a diameter of about 1 μm to about 10 μm. In some embodiments, the particle has a diameter of about 1 μm. In some embodiments, the particle has a surface area of about 1 μm² to about 1000 μm² (e.g., about 1 μm² to about 100 μm², about 1 μm² to about 20 μm²). In some embodiments, the particle has a number of about 10 to about 200,000 (e.g., about 100 to about 20000, e.g., about 10 to about 10000) second binding component associated with it. In some embodiments, wherein the display moiety comprises at least two different MHC class II molecules. In some embodiments, wherein the display moiety comprises at least one MHC class I molecule and at least one MHC class II molecule. In some embodiments, the display moiety comprises an HLA-A molecule. In some embodiments, the HLA-A molecule comprises a mutation that reduces its binding to CD8. In some embodiments, the HLA-A molecule comprises an HLA A2 heavy chain with a A245V mutation. In some embodiments, the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety comprises an MHC class II molecule selected from the group consisting of HLA-DP, HLA-DQ, and HLA-DR. In some embodiments, the peptide is associated with a cancer or tumor antigen. In some embodiments, the peptide is associated with a neoantigen. In some embodiments, the peptide is associated with an antigen of a pathogen. In some embodiments, the pathogen is a virus, bacteria of fungus. In some embodiments, the peptide is associated with an autoantigen. In some embodiments, the display moiety comprises at least two different peptides (e.g., at least 2, 5, 10, 20, 30, 40, 50, 100, 200, 500, or 1000 different peptides). In some embodiments, the at least two distinct peptides are associated with two different antigens. In some embodiments, the two distinct antigens comprise two distinct mutations of one or more genes (e.g., oncogenes) associated with a disease (e.g., a cancer). In some embodiments, the oncogene has a driven mutation. In some embodiments, the oncogene comprises one or more of KRAS, EGFR, POLE, TP53, SMAD, BRCA1, CDKN2A, TNN, MUC16, BRAF and ALK. In some embodiments, the oncogene comprises TP53, KRAS, MUC16, SMAD, CDKN2A and TNN. In some embodiments, the at least two different peptides overlap. In some embodiments, the at least two different peptides do not overlap. In some embodiments, the display moiety does not comprise a fluorochrome or fluorophore.

In some embodiments, the present application provides a display moiety comprising a particle (e.g., a spherical particle, e.g., a solid particle, e.g., a bead) associated with a plurality of MHC-peptide complexes, wherein the plurality of MHC-peptide complexes comprises at least or more than 2, 3, 4, 5, 6, 8, 9, 10, or 12 MHC-peptide complexes, wherein each of the MHC-peptide complex comprises an MHC molecule complexed with a peptide, wherein at least two MHC-peptide complexes are different, wherein the display moiety comprises at least two different MHC class II molecules. In some embodiments, at least one or each of the plurality of MHC-peptide complexes is an MHC-peptide monomer, MHC-peptide dimer, an MHC-peptide tetramer, an MHC-peptide pentamer, an MHC-peptide octamer, an MHC-peptide dextramer, or an MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer. In some embodiments, the MHC-peptide complex is attached to the particle via a binding pair comprising a first binding component attached to MHC-peptide complex or a unit thereof (i.e., a MHC-peptide monomer) and a second binding component bound to the particle. In some embodiments, the first binding component comprises a biotin, and the second binding component comprises streptavidin. In some embodiments, the particle has a diameter of about 0.5 μm to about 100 μm (e.g., 0.5 μm to about 100 μm, e.g., 1 μm to about 20 μm). In some embodiments, the particle has a diameter of about 1 μm to about 10 μm. In some embodiments, the particle has a diameter of about 1 μm. In some embodiments, the particle has a surface area of about 1 μm² to about 1000 μm² (e.g., about 1 μm² to about 100 μm², about 1 μm² to about 20 μm²). In some embodiments, the particle has a number of about 10 to about 200,000 (e.g., about 100 to about 20000, e.g., about 10 to about 10000) second binding component associated with it. In some embodiments, wherein the display moiety comprises at least one MHC class I molecule. In some embodiments, the display moiety comprises an HLA-A molecule. In some embodiments, the HLA-A molecule comprises a mutation that reduces its binding to CD8. In some embodiments, the HLA-A molecule comprises an HLA A2 heavy chain with a A245V mutation. In some embodiments, the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03. In some embodiments, the display moiety comprises an MHC class II molecule selected from the group consisting of HLA-DP, HLA-DQ, and HLA-DR. In some embodiments, the peptide is associated with a cancer or tumor antigen. In some embodiments, the peptide is associated with a neoantigen. In some embodiments, the peptide is associated with an antigen of a pathogen. In some embodiments, the pathogen is a virus, bacteria of fungus. In some embodiments, the peptide is associated with an autoantigen. In some embodiments, the display moiety comprises at least two different peptides (e.g., at least 2, 5, 10, 20, 30, 40, 50, 100, 200, 500, or 1000 different peptides). In some embodiments, the at least two distinct peptides are associated with two different antigens. In some embodiments, the two distinct antigens comprise two distinct mutations of one or more genes (e.g., oncogenes) associated with a disease (e.g., a cancer). In some embodiments, the oncogene has a driven mutation. In some embodiments, the oncogene comprises one or more of KRAS, EGFR, POLE, TP53, SMAD, BRCA1, CDKN2A, TNN, MUC16, BRAF and ALK. In some embodiments, the oncogene comprises TP53, KRAS, MUC16, SMAD, CDKN2A and TNN. In some embodiments, the at least two different peptides overlap. In some embodiments, the at least two different peptides do not overlap. In some embodiments, the display moiety does not comprise a fluorochrome or fluorophore.

### Support (e.g., Particle)

The support such as particles described herein can be any medium that provide a support for association with the MHC-peptide complexes described herein.

In some embodiments, the particle is solid. In some embodiments, the particle is not solid (e.g., hollow in the middle, e.g., having a dextran or polymer backbone). See e.g., J Immunol Methods. 2006 Mar. 20; 310 (1-2): 136-48.

In some embodiments, the particle is spherical (e.g., a bead). In some embodiments, for particles that a non-sphere shape. In those cases, in some embodiments, the diameter refers to minimum diagonal length; in some embodiments, diameter refers to the maximum diagonal length; in some embodiments, diameter refers to the average diagonal length.

In some embodiments, the particle has a diameter of at least 0.01, 0.05, 0.1, 0.2, 0.3, 0.4, 0.5, 0.6, 0.7, 0.8, 0.9, or 1 μm. In some embodiments, the particle has a diameter of about 0.01 to about 150 μm, or 0.01 to about 100 μm (such as about 0.1 to about 50 μm, such as about 0.5 μm to about 20 μm, such as about 0.5 μm to about 10 μm, such as about 0.5 μm to about 5 μm, such as about 0.5 μm to about 2.5 μm). In some embodiments, the particle has a diameter of about 0.1 to about 10 μm. In some embodiments, the particle has a diameter of about 0.5 to about 2 μm. In some embodiments, the particle has a diameter of about 1 μm.

In some embodiments, the particle has a surface area of about 0.01 μm² to about 400 μm² (such as about 0.1 μm² to about 20 μm², about 0.1 μm² to about 10 μm², or about 1 μm² to about 10 μm²). In some embodiments, the particle has a surface area of at least about 0.01 μm², about 0.1 μm², or about 1 μm².

In some embodiments, the particle has a number of at least about 2, 4, 5, 10, 12, 20, 50, 100, 200, 500, or 1000 binding component (e.g., second binding component as discussed above). In some embodiments, the particle has a number of about 2 to about 100000 (such as 13-100000, 20-100000, 50-100000, 100-10000) binding component associated with it. In some embodiments, the binding component (e.g., streptavidin) is directly conjugated to the particle.

In some embodiments, the particle is magnetic.

### MHC-Peptide Complexes

In some embodiments, the one or more of the plurality of MHC-peptide complexes are MHC-peptide monomers. In some embodiments, the one or more of the plurality of MHC-peptide complexes are MHC-peptide dimers. In some embodiments, the one or more of the plurality of MHC-peptide complexes are MHC-peptide trimers. In some embodiments, the one or more of the plurality of MHC-peptide complexes consist of MHC-peptide monomers, dimers and/or trimers. In some embodiments, the one or more of the plurality of MHC-peptide complexes are MHC-peptide tetramers. In some embodiments, the one or more of the plurality of MHC-peptide complexes are MHC-peptide pentamers. In some embodiments, the one or more of the plurality of MHC-peptide complexes are MHC-peptide octamers. In some embodiments, the one or more of the plurality of MHC-peptide complexes are MHC-peptide dextramers. In some embodiments, the one or more of the plurality of MHC-peptide complexes are MHC-peptide dodecamer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, the one or more of the plurality of MHC-peptide complexes comprise a mixture of one or more of MHC-peptide monomers, one or more of MHC-peptide tetramers, one or more of MHC-peptide dimers, one or more of MHC-peptide pentamers, one or more of MHC-peptide octamers, one or more of MHC-peptide dextramers, and/or one or more of MHC-peptide dodecamers. In some embodiments, the one or more of the plurality of MHC-peptide complexes comprise a mixture of one or more of MHC-peptide monomers and one or more of MHC-peptide tetramers. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

In some embodiments, the plurality of MHC-peptide complexes comprise at least 20 MHC-peptide complexes. In some embodiments, the plurality of MHC-peptide complexes comprise at least 50 MHC-peptide complexes. In some embodiments, the plurality of MHC-peptide complexes comprise at least 100 MHC-peptide complexes. In some embodiments, the plurality of MHC-peptide complexes comprise at least 500 MHC-peptide complexes. In some embodiments, the plurality of MHC-peptide complexes comprise at least 1000 MHC-peptide complexes. In some embodiments, the plurality of MHC-peptide complexes comprise about 10-100000 (such as 20-100000, 50-10000, 100-10000) MHC-peptide complexes.

In some embodiments, one or more, or each of the plurality of the MHC-peptide complex is attached to the support (e.g., the particle) directly (i.e., no intermediate sperate molecule in between). In some embodiments, one or more, or each of the plurality of the MHC-peptide complex is attached to the support (e.g., the particle) via a binding pair comprising a first binding component attached to MHC-peptide complex or a unit thereof (i.e., a MHC-peptide monomer) and a second binding component bound to the particle.

Exemplary binding pairs comprise any binding pairs that selectively bind to each other. In some embodiments, the binding components are solely for promoting the binding between the MHC-peptide complex and the support (e.g., the particle). In some embodiments, the first binding component comprises biotin or a synthetic mimic of biotin (e.g., 2-iminobiotin), and the second binding component comprises streptavidin or a synthetic mimic of streptavidin, or vice versa. See e.g., Chem Soc Rev. 2017 May 9; 46 (9): 2391-2403. In some embodiments, the first binding component comprises a biotin, and the second binding component comprises streptavidin. In some embodiments, the binding components are any suitable moieties known in the art (such as, thiol, maleimide, cyclodextrin, amine, adamantine, carboxy, azide, and alkyne). In some embodiments, the two binding components comprise a binding pair that reacts via a click chemistry reaction. Exemplary reactions include: Strain-promoted azido-alkyne cycloaddition (SPAAC), Cu-mediated azido-alkyne cycloaddition (CuAAC), Staudinger ligation, Boronic acid-mediated facile assembly involving aromatic aminothiols, Boronic acid-mediated facile assembly involving amines or oximes, Boronic acid-mediated facile assembly involving hydrazides, Boronic acid-mediated facile assembly involving α-aminothiols, Boronic acid-mediated facile assembly involving amines, Strained alkyne-tetrazine cycloaddition, Strain-promoted alkyne-nitrone cycloaddition (SPANC), Strain-promoted alkyne-tetrazole cycloaddition, Sydnone-strained alkyne reaction. See e.g., Acta Pharm Sin B. 2023 May; 13 (5): 1990-2016.

### MHC Molecules

MHC I and II molecules present protein fragments to CD8+ and CD4+ T cells, respectively. These molecules are essential for cell-mediated immunity and therefore appeared at the inception of the adaptive immune system. For their construction they used two Ig-domains topped by two parallel alpha helixes resting on a platform of beta-pleated sheets. This capital structure generated a peptide-binding groove between the alpha helixes, which is ‘evolutionarily speaking’ likely borrowed from earlier chaperone structures. Yet MHC I and MHC II molecules are unique in the proteome because of their extreme polymorphism (>10,000 different alleles of MHC I molecules have been identified thus far). This has interesting consequences. Polymorphic residues on the top alpha helixes interact with the TCR and are the basis for the specificity of TCRs for both an antigen peptide plus a particular allelic form of an MHC molecule (a phenomenon called MHC restriction). Polymorphic residues in the MHC peptide binding groove change the nature and location of so-called pockets. These variable pockets are filled by complementary variable amino acid side chains of peptides (so-called anchor residues), with the effect that different fragments from a defined antigen are presented by different polymorphic MHC molecules. Yet, next to the anchor residues, most other amino acids in a peptide fill a free space and can be (almost) any of the 20 amino acids. By having pockets with specificity for only a few side chains and allowing the remaining 6-10 amino acids to vary between all possibilities, each kind of MHC molecules can present a very large repertoire of peptides. Moreover, by having 3 to 6 different MHC I as well as 3 to 12 different MHC II molecules (the exact number depending on how many different MHC alleles were inherited from one's parents and how the MHC II subunits paired), cells can present a large fraction of the universe of peptides, although not all sequences. See e.g., Trends Immunol. 2016 November; 37 (11): 724-737.

In theory then, MHC I molecules can present a peptidome of around 6×20⁽⁶⁻⁷⁾ different peptides, and MHC II ca display up to 12×20⁽¹⁰⁾ peptides. In actuality, such a large array of peptides cannot all be presented because there are only around 200,000 MHC I and 20,000 MHC II molecules on cells such as B and T cells per cell. Moreover, since some peptides are present in high number (from highly expressed proteins), the real number of different peptides presented by one cell is likely less than 10,000. Importantly, when a pathogen alters a critical anchor residue in one of its antigenic epitopes, it may prevent presentation of this antigen in one individual but not in another person with different MHC molecules that will simply select different peptides from the same pathogen. Therefore, MHC polymorphism is good for the survival of the population and not necessarily the individual. See e.g., Trends Immunol. 2016 November; 37 (11): 724-737.

The MHC molecules described herein can be any MHC molecules. See e.g., Cell Mol Immunol. 2015 March; 12 (2): 139-53.

In some embodiments, the MHC molecule is a MHC class I molecule.

In some embodiments, the MHC class I molecule is selected from the group consisting of HLA-A, HLA-B, HLA-C, and HLA-D. In some embodiments, the MHC class I molecule is selected from the group consisting of HLA-A, HLA-B, and HLA-C. In some embodiments, MHC class I molecule comprises a HLA-A molecule. In some embodiments, the HLA-A molecule comprises a mutation that reduces its binding to CD8, further optionally where the HLA-A molecule comprises a HLA A2 heavy chain with a A245V mutation. It was found that this mutation does not result in loss of reactivity towards some of the specific T cells, but in fact with result in a decreased the background staining.

In preferred embodiments, the MHC class I molecule is selected from the group consisting HLA-A*24: 02, HLA-A*11: 01, HLA-A*02: 01, and HLA-A*03: 01. In preferred embodiments, the MHC class I molecule comprises multiple kinds of MHC class I molecules comprising HLA-A*24: 02, HLA-A*11: 01, HLA-A*02: 01, and HLA-A*03: 01. In another preferred embodiments, the MHC class I molecule comprises multiple kinds of MHC class I molecules comprising HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03.

In some embodiments, the HLA molecule has a His-tag fused to the C-terminus. In some embodiments, the HLA molecule has a polypeptide (e.g., a GS linker, e.g., a G4S linker) that has a length of about 5-15, 7-13, 8-12, 9-11 or 10 amino acids fused to the C-terminus.

In some embodiments, the antigenic peptide complexed with MHC I molecule is about 8 to about 10 amino acids long. In some embodiments, the antigenic peptide is at least 8 (e.g., 8, 9, or 10) amino acids long.

In some embodiments, the MHC molecule is a recombinant MHC I molecule.

In some embodiments, the MHC molecule is a MHC class II molecule.

In some embodiments, the MHC class II molecule is selected from the group consisting of HLA-DR, HLA-DQ, and HLA-DF. In some embodiments, the MHC class II molecule is selected from the group consisting of HLA-DQ and HLA-DR. In some embodiments, the antigenic peptide that complexed with a MHC class II molecule is about 10 to about 20 amino acids long. In some embodiments, the antigenic peptide is at least 10 (e.g., 10, 11, 12, 13, 14, 15, 16, 17, 18, 19, or 20) amino acids long.

In some embodiments, the MHC molecule is a recombinant MHC II molecule.

In some embodiments, the MHC molecule comprise both an MHC I molecule and an MHC II molecule.

In preferred embodiments, the MHC molecule matches with at least one HLA type of the individual from where the sample is obtained. For example, a MHC I molecule comprising HLA-A*24: 02 or HLA-A*11: 01 is used when the individual has both HLA-A*24: 02 and HLA-A*11: 01. Patient-specific NGS data from WGS, WES, or RNA-seq can be used to predict HLA types with computational tools such as Optiptype and Polysolver (polymorphic loci resolver). See e.g., Szolet et al., Bioinformatics 30, 3310-3316, e.g., Shukla et al., Nat. Biotechnol. 33, 1152-1158. Reads can be selected from the NGS data that potentially derived from the HLA region and then they can be fully aligned to a full-length genomic library of all known HLA alleles. See e.g., Nucleic Acids Res. 41, D1222-D1227.

In some embodiments, the MHC molecule is coupled with a chaperon molecule prior to being complexed to the antigenic peptide. See e.g., Overall et al., Nat Commun. 2020 Apr. 20; 11 (1): 1909.

In some embodiments, the display moiety comprises an HLA-A molecule, an HLA-B molecule and/or an HLA-C molecule. In some embodiments, the display moiety comprises an HLA-A molecule. In some embodiments, the HLA-A molecule comprises a mutation that reduces its binding to CD8. In some embodiments, the HLA-A molecule comprises an HLA A2 heavy chain with a A245V mutation.

In some embodiments, the display moiety comprises an MHC class II molecule. In some embodiments, the MHC class II molecule is selected from the group consisting of an HLA-DO molecule, an HLA-DM molecule, an HLA-DP molecule, an HLA-DQ molecule and an HLA-DR molecule. In some embodiments, the MHC class II molecule is selected from the group consisting of an HLA-DP molecule, an HLA-DQ molecule and an HLA-DR molecule. In some embodiments, the display moiety comprises an MHC class II molecule selected from the group consisting of an HLA-DQ molecule and an HLA-DR molecule.

In some embodiments, the display moiety described herein comprises at least two different MHC molecules.

In some embodiments, the at least two different MHC molecules comprises two different MHC class I molecules. In some embodiments, the two different MHC class I molecules comprise an HLA-A molecule and at least one of an HLA-B molecule and an HLA-C molecule. In some embodiments, the two different MHC class I molecules comprise an HLA-A molecule and an HLA-B molecule. In some embodiments, the two different MHC class I molecules comprise an HLA-A molecule and an HLA-C molecule. In some embodiments, the two different MHC class I molecules comprise an HLA-B molecule and an HLA-C molecule. In some embodiments, the two different MHC class I molecules comprise a) an HLA-A molecule, b) an HLA-B molecule and c) an HLA-C molecule.

In some embodiments, the MHC multimer comprises at least two MHC class I molecules that are at least two different HLA-A molecules. In some embodiments, the at least two different HLA-A molecules are selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03.

In some embodiments, the at least two different MHC molecules comprises two different MHC class II molecules. In some embodiments, the two different MHC class II molecules comprise at least two MHC class molecules selected from the group consisting of an HLA-DP molecule, an HLA-DQ molecule and an HLA-DR molecule. In some embodiments, the two different MHC class II molecules comprise an HLA-DP molecule and an HLA-DQ molecule. In some embodiments, the two different MHC class II molecules comprise an HLA-DP molecule and an HLA-DR molecule. In some embodiments, the two different MHC class II molecules comprise an HLA-DR molecule and an HLA-DQ molecule. In some embodiments, the two different MHC class II molecules comprise an HLA-DP molecule, an HLA-DQ molecule, and an HLA-DR molecule.

In some embodiments, the at least two different MHC molecules comprises both an MHC class I molecule and an MHC class II molecule. In some embodiments the at least two different MHC molecules comprise a) an HLA-A molecule and b) an MHC class II molecule selected from the group consisting of an HLA-DP molecule, an HLA-DQ molecule and an HLA-DR molecule. In some embodiments the at least two different MHC molecules comprise a) an HLA-A molecule and b) an HLA-DP molecule. In some embodiments the at least two different MHC molecules comprise a) an HLA-A molecule and b) an HLA-DQ molecule. In some embodiments the at least two different MHC molecules comprise a) an HLA-A molecule and b) an HLA-DR molecule.

In some embodiments, the display moiety comprises two or more (e.g., at least 2, 3, 4, 5, 6, 7, 8, 9, 10, 20, 30, 40, 50, 60, 70, 80, 90, 100, 1000, 10,000, or 100,000) antigenic peptides. In some embodiments, the display moiety comprises four antigenic peptides. In some embodiments, the two or more antigenic peptides in the display moiety are the same or similar. In some embodiments, the two or more antigenic peptides in the display moiety are distinct. In some embodiments, at least one of the two or more antigenic peptides is a truncal antigenic peptide.

In some embodiments, the display moiety comprises an MHC/peptide monomer, dimer, trimer, and/or tetramer. In some embodiments, MHC/peptide complexes are assembled into monomers, dimers, trimers, and/or tetramers, comprising 1, 2, 3, or 4 MHC/peptide complexes bound to a display moiety. In some embodiments, the MHC/peptide complex further comprises a detectable label. The detectable label is a fluorophore, such as phycoerythrin (PE), allophycocyanin (APC) or any fluorophore known in the art. In some embodiments, the MHC/peptide complex does not comprise a detectable label.

In some embodiments, the MHC/peptide complex is assembled into a multimer (such as, dimer, trimer, tetramer, pentamer, hexamer, or high order multimer). In some embodiments, the multimer can comprise at least 2, 3, 4, 5, 6, 7, 8, 9, 10, 11, or 12 MHC/peptide complexes. In some embodiments, a high throughput peptide-MHC (pMHC) tetramer library is constructed. See e.g., Overall et al., Nat Commun. 2020 Apr. 20; 11 (1): 1909.

In some embodiments, the display moiety further comprises a barcode (e.g., a DNA barcode). In some embodiments, each of the one or more display moieties comprises a unique barcode (e.g., a unique DNA barcode). See e.g., Overall et al., Nat Commun. 2020 Apr. 20; 11 (1): 1909.

In some embodiments, the display moiety comprises a particle. In some embodiments, the particle is selected from the group consisting of a surface, a nanoparticle, a bead, and a polymer. In some embodiments, the particle is a magnetic nanoparticle, e.g. for isolation using a magnet. See e.g., Peng et al., Cell Rep. 2019 Sep. 3; 28 (10): 2728-2738. e7. In some embodiments, the magnetic particle comprises magnetic iron oxide. In some embodiments, the particle is a polystyrene nanoparticle, e.g., for isolation by gravity. In some embodiments, the particle is an agarose bead. In some embodiments, the particle is a sepharose bead. In some embodiments, the particle is a dextran particle. In some embodiments, the particle is a biotinylated dextran or a streptavidin-coated dextran. The particle in the display moiety is not the same as the solid support or substrate used to immobilize the immune cells as described herein.

In some embodiments, the particle is detectable. In some embodiments, the particle is fluorescent. In some embodiments, the particle is attached directly or indirectly to a fluorophore. In some embodiments, the particle is modified with an attachment moiety for attaching additional molecules.

In some embodiments, the antigenic peptide or MHC is directly attached to the particle. In some embodiments, the antigenic peptide or MHC is attached to the particle via a binding pair comprising a first binding component attached to the antigenic peptide and a second binding component bound to the particle. In some embodiments, the binding components are any suitable moieties known in the art (such as, thiol, maleimide, cyclodextrin, amine, adamantine, carboxy, azide, and alkyne).

In some embodiments, multiple display moieties (e.g., MHC/peptide complexes) are attached to a single particle.

In some embodiments, the display moiety comprises a cell (e.g., an antigen presenting cell, e.g., a dendritic cell, e.g., a macrophage). In some embodiments, the cell comprises a polynucleotide encoding the antigenic peptide (e.g., a truncal antigenic peptide). In some embodiments, the polynucleotide encodes a plurality of antigenic peptides. In some embodiments, the plurality of antigenic peptides are displayed on the surface of the cell (e.g., antigen presenting cells). The cells used for display antigenic peptides are different from the immune cells in the sample described herein. In some embodiments, the plurality of antigenic peptides are displayed on the surface of the cell in complex with an MHC molecule.

In some embodiments, the cell is obtained from the individual. In some embodiments, the cell has at least one (or two) same HLA type as that of the individual. For example, if the individual has HLA-A*24: 02, the cell in the display moiety also has HLA-A*24: 02.

In some embodiments, the display moiety further comprises a detectable label. In some embodiments, the detectable label is a fluorophore. In some embodiments, the display moiety is itself fluorescent or is attached to a fluorophore directly or indirectly. In some embodiments, the fluorophore is a phycoerythrin (PE), allophycocyanin (APC) or any fluorophore known in the art.

### Antigenic Peptides (i.e., Peptides)

Peptides described herein can be derived from exogenous antigens (e.g., an antigen from a pathogen) or endogenous antigens (e.g., an antigen associated with a cancer cell, e.g., a neoantigen, e.g., an autoantigen).

The size of a antigenic peptide can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In preferred embodiments, the antigenic peptide molecules are equal to or less than 50 amino acids.

In some embodiments, the antigenic peptide complexed with a MHC Class I molecule has 15 residues or less in length and usually consist of between about 8 and about 11 residues, particularly 9 or 10 residues. In some embodiments, the antigenic peptides complexed with a MHC Class II molecule has 6-30 residues.

If desirable, a longer peptide can be designed in several ways. For example, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each.

In some embodiments, the one or more antigenic peptides are about 8-50 amino acids in length. In some embodiments, it is about 8-10 amino acids in length. In some embodiments, it is greater than 10 amino acids in length, greater than 15 amino acids in length, greater than 20 amino acids in length, or greater than 30 amino acids in length. In some embodiments, it is about 24-40 amino acids in length.

In some embodiments, the one or more antigenic peptides used in the bait compositions described herein are further optimized based upon one or more selection criteria.

In some embodiments, the antigenic peptides are further selected based on their likelihood to be processed and/or presented on the cell surface HLA molecules. In some embodiments, in silico prediction algorithms (such as any of the algorithms described herein) is used as the basis for the selection. In some embodiments, immunopeptidomics analysis is used as the basis for the selection.

Computational algorithms such as NetMHC (See e.g., Andreatta et al., Bioinformatics 32, 511-517, 2016), NetMHCpan (See e.g., Rammensee et al., Immunogenetics 50, 213-219, 1999), and MHCflurry (O'Donnell et al., Cell Syst. 7, 129-132. e124, 2018) trained on large in vitro experimental datasets can be used to prioritize candidate neoantigens that bind to the predicted HLA types with high affinity. For example, Neopepsee and pVAC-Seq are representative analysis pipelines for tumor somatic mutations (Hundal et al., Genome Med. 8, 11, 2016; Kim et al., Ann. Oncol. 29, 1030-1036, 2018). Recently, a new prediction model-EDGE based on tumor HLA peptide mass spectrometry (MS) datasets has increased the positive predictive value up to nine-fold. (Bulik-Sullivan et al., Nat. Biotechnol. 18: 4313).

In some embodiments, the one or more antigenic peptides are selected based upon its binding affinity to a) an MHC molecule and/or b) a cognate TCR molecule.

In some embodiments, the antigenic peptide has a binding affinity that is less than 5000 nM (IC50) to an MHC molecule. In some embodiments, the antigenic peptide has a binding affinity of about 500 nM to 5000 nM (IC50) to an MHC molecule. In some embodiments, the antigenic peptide has a binding affinity that is less than 500 nM (IC50) to an MHC molecule. In some embodiments, the antigenic peptide has a binding affinity of about 250 nM to 500 nM IC50 to an MHC molecule. In some embodiments, the antigenic peptide has a binding affinity that is less than 250 nM (IC50) to an MHC molecule. In some embodiments, the antigenic peptide has a binding affinity that is less than 100 nM (IC50) to an MHC molecule. In some embodiments, the antigenic peptide has a binding affinity of about 50 nM to 500 nM IC50 to an MHC molecule. In some embodiments, the antigenic peptide has a binding affinity that is less than 50 nM (IC50) to an MHC molecule. In some embodiments, the antigenic peptide has a binding affinity of about 1 nM to 50 nM IC50 to an MHC molecule.

In some embodiments, the antigenic peptide has a binding affinity that is less than 5000 nM (IC50) to a cognate TCR molecule. In some embodiments, the antigenic peptide has a binding affinity of about 500 nM to 5000 nM (IC50) to a cognate TCR molecule. In some embodiments, the antigenic peptide has a binding affinity of about 50 nM to 500 nM IC50 to a cognate TCR molecule. In some embodiments, the antigenic peptide has a binding affinity of about 1 nM to 50 nM IC50 to a cognate TCR molecule.

In some embodiments, the antigenic peptide is selected based upon its hydrophobic status. In some embodiments, the antigenic peptide is hydrophobic. In some embodiments, the antigenic peptide has a high content of aromatic residues. In some embodiments, the antigenic peptide has at least about 10%, 20%, 30%, or 40% aromatic residues.

In some embodiments, the antigenic peptide has a binding affinity of about 1 nM to about 5000 nM (e.g., about 1 nM to about 50 nM, about 50 nM to about 500 nM, about 500 nM to about 5000 nM) to an MHC molecule, a binding affinity of about 1 nM to about 5000 nM (e.g., about 1 nM to about 50 nM, about 50 nM to about 500 nM, about 500 nM to about 5000 nM) to a cognate TCR molecule, a mutation relative to a wildtype peptide, optionally at the third amino acid position counting from the N-terminus, is hydrophobic, and has high content of aromatic residues.

In some embodiments, the antigenic peptide has low immunogenicity. Immunogenicity of the antigenic peptide can be predicted by algorithm developed for this purpose. See e.g., Riley et al., Front Immunol. 2019 Aug. 28; 10: 2047; e.g., Schmidt et al., Cell Rep Med. 2021 Feb. 6; 2 (2): 100194.

In some embodiments, the antigenic peptide may be flanked by universal sequences or portions thereof. In some embodiments, the universal sequences or portions allow for rapid, high throughput methods for replacing or inserting the antigenic peptide encoding nucleotide in the polynucleotide MHC template.

In some embodiments, the antigenic peptides further comprise a unique defined barcode sequence operably associated with the identity of each distinct polypeptide. In some embodiments, the unique defined barcodes provide an antigen-specific sequence for identification during the analysis of the immune cell. See e.g., Peng et al., Cell Rep. 2019 Sep. 3; 28 (10): 2728-2738. e7.

In some embodiments, the peptide is associated with a cancer or tumor antigen. In some embodiments, the peptide is associated with a neoantigen.

In some embodiments, the peptide is associated with an antigen of a pathogen. In some embodiments, the pathogen is a virus, bacteria of fungus.

In some embodiments, the peptide is associated with an autoantigen.

### Neoantigenic Peptides

Neoantigens can be formed via various mechanisms. Non-synonymous somatic mutations, which can alter amino acid coding sequences, are the main cause of neo-epitopes. Except for somatic non-synonymous protein-altering mutations, tumor neoantigens can be generated from alternative splicing variations. Multiple computational methods and databases have been developed to identify alternative splicing events from RNA-seq data, such as SplAdder and CancerSplicingQTL2. See e.g., Kahles et al., Bioinformatics 32 1840-1847; Tian et al., Nucleic Acids Res. 47 D909-D916. A computational strategy was developed to identify neoepitopes generated from intron retention events in tumor transcriptomes and confirmed that these neoepitopes were processed and presented on MHC-I. See Smart et al. Nat. Biotechnol. 36 1056-1058.

### Detection and Screening of Neoantigens

Various methods are available to detect and screen neoantigens. Sandwich immunoassays in the miniaturized system could successfully identify tumor antigens in sera samples extracted from patients. See e.g., Pollard et al., Proteomics Clin. Appl. 1 934-952 (2007); Yang et al., Biosens. Bioelectron. 40 385-392 (2013). Another tool named Serologic Proteome analysis (SERPA) or 2-D western blots, consists of the isoelectric focusing (IEF) gel run in the first dimension and SDS-PAGE gel run in the second dimension. SERPA separates the proteins in the gel by their isoelectric point (IP) and molecular mass and then transfers the proteins from the gel to a carrier membrane to screen antibodies. Finally, the antigenic protein spots can be identified by MS. See e.g., Tjalsma et al., Proteomics Clin. Appl. 2 167-180 (2008). This approach has been used to identify antigens in different tumor types. Serological analysis of recombinant cDNA expression libraries (SEREX), which combines serological analysis with antigen cloning techniques, is a widely used technique to explore tumors' antigen repertoire. SEREX first construct a cDNA library from cancer cell lines or fresh tumor samples, then screen the cDNA library with autologous sera of cancer patients, and finally sequence the immune-reactive clones. SEREX have identified a variety of tumor antigens including CTAs, differentiation antigens, mutational antigens, splice-variant antigens and overexpressed antigens. See e.g., Chen et al., Proc. Natl. Acad. Sci. U.S.A. 94 1914-1918 (1997). Furthermore, other methods such as Multiple Affinity Protein Profiling (MAPPing) and nanoplasmonic biosensor have also been developed to identify tumor antigens. See e.g., Lee et al., Biosens. Bioelectron. 74 341-346 (2015).

In some embodiments, the one or more neoantigenic peptides described herein are obtained from a neoantigenic database (such as any of the neoantigenic databases described herein). For example, Tan et al constructed a manually curated database (“dbPepNeo”) for human tumor neoantigen peptides based upon the four criterias as below: (i) peptides were isolated from human tumor tissues or cell lines, (ii) peptides contained non-synonymous mutations in amino acid sequence, (iii) Peptides can be bound by HLA-I molecules, (iv) Peptides can induce CD8+ T cell responses. See Tan et al., Database (Oxford). 2020 Jan. 1; 2020: baaa004. Xia et al constructed another database, NEPdb, which provides pan-cancer level predicted HLA-I neoepitopes derived from 16, 745 shared cancer somatic mutations, using state-of-the-art predictors. See Xia et al., Front Immunol. 2021; 12: 644637. Wu et al. developed a comprehensive tumor-specific neoantigen database (TSNAdb v1.0), based on pan-cancer immunogenomic analyses of somatic mutation data and human leukocyte antigen (HLA) allele information for 16 tumor types with 7748 tumor samples from The Cancer Genome Atlas (TCGA) and The Cancer Immunome Atlas (TCIA). See Wu et al., enomics Proteomics Bioinformatics. 2018 August; 16 (4): 276-282.

In some embodiments, the one or more neoantigenic peptides are obtained from analyzing the biological information of the individual (such as a patient who had a cancer). In some embodiments, the neoantigenic peptides are obtained from a computational analysis of a cancer patient's tumor genome. See e.g., Roudko et al. Front Immunol. 2020; 11: 27. In some embodiments, the neoantigenic peptides are obtained from a computational analysis of a cancer patient's transcriptome. See e.g., Caushi et al., Nature. 2021 August; 596 (7870): 126-132. In some embodiments, the neoantigenic peptides are obtained from a computational analysis of a cancer patient's proteome. See e.g., Wen et al. Nat Commun. 2020 Apr. 9; 11 (1): 1759.

In some embodiments, the neoantigenic peptides are selected from patient data. In some embodiments, the patient data is derived from data from a group of patients having a particular type of cancer (e.g., any of the cancers described here). In some embodiments, the patient data is derived from data from a group of patients having any cancer. In some embodiments, the group of patients are from the same sex (e.g., male or female). In some embodiments, the group of patients are from the same ethnicity. In some embodiments, the group of patients bear one or more biomarkers (e.g., an aberration in a particular gene, e.g., KRAS, e.g., PTEN).

In some embodiments, the one or more neoantigenic peptides are derived from any polypeptide known to or have been found to contain a tumor specific mutation. Suitable polypeptides from which the neoantigenic peptides can be derived can be found for example in various databases available in the field (e.g., COSMIC database). These databases curate comprehensive information on somatic mutations in human cancer. In some embodiments, the peptide contains a tumor specific mutation. In some embodiments, the tumor specific mutation is a driver mutation for a particular cancer type.

In some embodiments, the neoantigenic peptide is selected based upon its mutational status. In some embodiments, the neoantigenic peptide has a mutation at the third amino acid position counting from the N-terminus, relative to a wildtype peptide. In some embodiments, the neoantigenic peptides may comprise two or more (such as at least 2, 3, 4, or 5) somatic mutations.

In some embodiments, a library of tumor specific neoantigenic peptides are synthesized (e.g., based upon one or a group of patients as described above). In some embodiments, the neoantigenic peptides are obtained by exome high throughput sequencing and prescreened with epitope prediction algorithms.

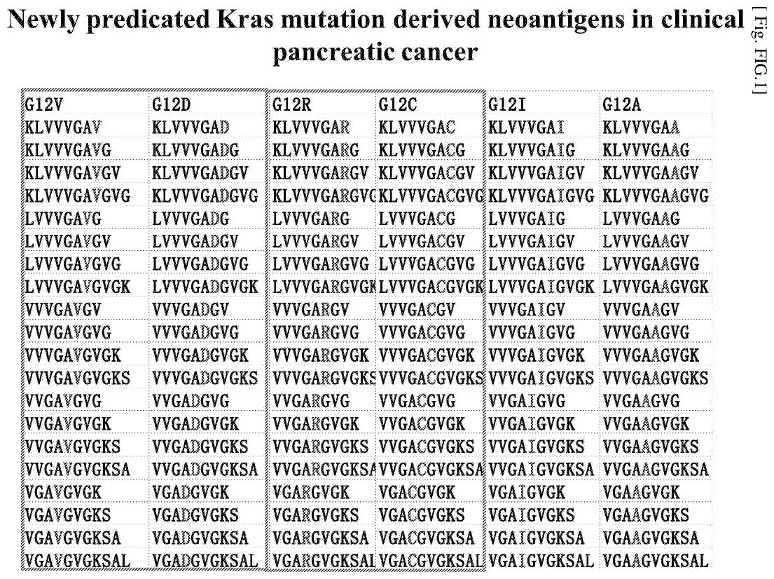

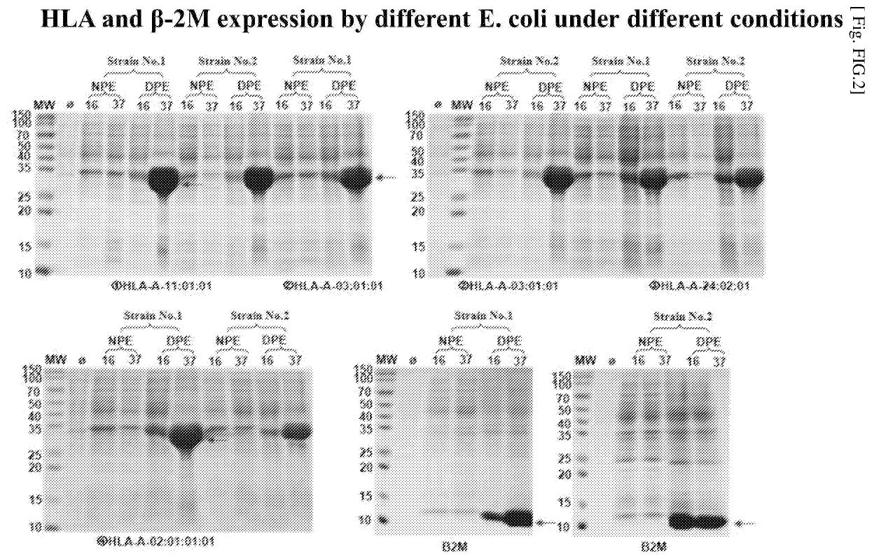

In some embodiments, the methods involve constructing a plurality of neoantigen peptide peptides (e.g., building a library, such as in Example 1 and FIG. 1). In some embodiments, the plurality of neoantigen peptides are based upon a known mutation (e.g., Kras mutation). In some embodiments, the plurality of neoantigen peptides based upon one known mutation (e.g., a point mutation, e.g., Kras G12D, G12V, G12C, G12R, G12A, or G12I) comprises at least about 5, 10, 12, 15, 18 or 20 distinct neoantigen peptides. In some embodiments, the plurality of neoantigen peptides based upon one known mutation comprise at least about 5, 10, 12, 15, 18 or 20 distinct neoantigen peptides that a) have a binding affinity of about 1 nM to about 5000 nM to an MHC molecule; b) have a binding affinity of about 1 nM to about 5000 nM to a cognate TCR molecule; c) have a mutation relative to a wildtype peptide, optionally at the third amino acid position counting from the N-terminus; d) are hydrophobic; and/or e) have high content of aromatic residues.

### Peptides in a Display Moiety

In some embodiments, the display moiety comprises at least two distinct peptides (e.g., at least 3, 4, 5, 10, 12, 15, 20, 50, or 100 distinct peptides). In some embodiments, the display moiety comprises at least five distinct peptides. In some embodiments, the display moiety comprises at least 10 distinct peptides. In some embodiments, the at least two distinct peptides in the display moiety do not overlap. In some embodiments, the at least two distinct peptides in the display moiety overlaps.

In some embodiments, the at least two distinct peptides are associated with two different antigens. In some embodiments, the at least two distinct peptides are associated with at least three, four, five, six, ten, or twelve different antigens. In some embodiments, the at least two distinct peptides are associated with at least about 10, 20, 30, 40, 50, 60, 70, 80, 90, or 100 different antigens.

In some embodiments, the at least two distinct antigens comprise two distinct mutations of one or more genes associated with a disease. In some embodiments, the at least two distinct antigens comprise 3, 4, 5, 6, 10, 12, 15, 20, 30, 40, 50, 60, 70, 80, 90, or 100 distinct mutations of one or more genes associated with a disease. In some embodiments, the disease is a cancer. In some embodiments, the gene is an oncogene. In some embodiments, the disease is not a cancer or tumor (e.g., an infection associated with a pathogen, e.g., an autoimmune disease).

### Multiple Display Moieties or Bait Composition

The present application also provides bait compositions that comprise a plurality of the display moieties described here. In some embodiments, the bait composition comprises at least two different display moieties.

In some embodiments, the bait composition comprises at least about 20, 50, 100, 500, 1000, 2000, 3000, 4000, 5000, 6000, 7000, 8000, 9000, or 10000 different MHC-peptide complexes. In some embodiments, the bait composition comprises a) at least two different MHC molecules (such as at least three, four, five, or six) different MHC molecules, and b) at least about 20, 50, 100, 150, 200, 250, 300, 400, 500, 750, or 1000 different peptides.

In some embodiments, the bait composition comprises a) at least two different (or at least three different) different MHC I molecules, and b) at least about 20, 50, 100, 150, 200, or 250 different peptides. In some embodiments, the at least two or three different MHC molecules comprise an HLA-A molecule and at least one of an HLA-B molecule and an HLA-C molecule. In some embodiments, the at least two or three different MHC molecules comprise an HLA-A molecule, an HLA-B molecule, and an HLA-C molecule. In some embodiments, the at least two or three different MHC molecules comprise two or more different HLA molecules. In some embodiments, the bait composition comprises HLA-A*24: 02, HLA-A*11: 01, HLA-A*02: 01, or HLA-A*03: 01. In some embodiments, the bait composition comprises HLA-A*24: 02, HLA-A*11: 01, HLA-A*02: 01, and HLA-A*03: 01.

In some embodiments, the bait composition comprises a) at least two, three, or four HLA-A molecules, and b) at least about 20, 50, 100, 150, 200, or 250 different peptides, wherein the two, three or four HLA-A molecules are selected from the group consisting of HLA-A*24: 02, HLA-A*11: 01, HLA-A*02: 01, and HLA-A*03: 01.

In some embodiments, the bait composition comprises a) at least two different (or at least three different) different MHC II molecules, and b) at least about 20, 50, 100, 150, 200, or 250 different peptides. In some embodiments, the bait composition comprises an HLA-DM molecule, an HLA-DO molecule, an HLA-DP molecule, an HLA-DQ molecule, or an HLA-DR molecule. In some embodiments, the bait composition comprises an HLA-DP molecule, an HLA-DQ molecule, and an HLA-DR molecule.

In some embodiments, the bait composition comprises a) at least one MHC class I molecule and at least one MHC class II molecule, and b) at least about 20, 50, 100, 150, 200, or 250 different peptides. In some embodiments, the bait composition comprises a) at least two different MHC class I molecules and at least one MHC class II molecule, and b) at least about 20, 50, 100, 150, 200, or 250 different peptides. In some embodiments, the bait composition comprises a) at least one MHC class I molecule and at least two different MHC class II molecules, and b) at least about 20, 50, 100, 150, 200, or 250 different peptides. In some embodiments, the bait composition comprises a) at least two different MHC class I molecules and at least two MHC class II molecules, and b) at least about 20, 50, 100, 150, 200, or 250 different peptides. In some embodiments, the MHC class I molecule or molecules comprise an HLA-A molecule, an HLA-B molecule and/or an HLA-C molecule. In some embodiments, the MHC class I molecule or molecules comprise an HLA-A molecule, an HLA-B molecule and an HLA-C molecule. In some embodiments, the MHC class I molecule or molecules comprise two or more different HLA-A molecules. In some embodiments, the bait composition comprises HLA-A*24: 02, HLA-A*11: 01, HLA-A*02: 01, or HLA-A*03: 01. In some embodiments, the one or more MHC class II molecules comprise an HLA-DM molecule, an HLA-DO molecule, an HLA-DP molecule, an HLA-DQ molecule, or an HLA-DR molecule. In some embodiments, the one or more MHC class II molecules comprises an HLA-DP molecule, an HLA-DQ molecule, and an HLA-DR molecule.

In some embodiments, the bait composition comprises a) at least three MHC class I molecules and at least one MHC class II molecules, and b) at least about 20, 50, 100, 150, 200, or 250 different peptides. In some embodiments, the bait composition comprises a) at least one MHC class I molecule and at least three MHC class II molecules, and b) at least about 20, 50, 100, 150, 200, or 250 different peptides. In some embodiments, the bait composition comprises a) at least three MHC class I molecules and at least three MHC class II molecules, and b) at least about 20, 50, 100, 150, 200, or 250 different peptides. In some embodiments, the at least three MHC class I molecules comprise an HLA-A molecule and at least one of an HLA-B molecule and an HLA-C molecule. In some embodiments, the at least three MHC class I molecules comprise an HLA-A molecule, an HLA-B molecule and an HLA-C molecule. In some embodiments, the at least three MHC class I molecules comprise two or three or more different HLA-A molecules. In some embodiments, the bait composition comprises HLA-A*24: 02, HLA-A*11: 01, HLA-A*02: 01, or HLA-A*03: 01. In some embodiments, the three or more MHC class II molecules comprise an HLA-DP molecule, an HLA-DQ molecule, and an HLA-DR molecule.

In some embodiments, the peptides in the bait composition are associated with at least 25, 50, 75, 100, 125, 150, 175, 200, 225, or 250 different antigens.

In some embodiments, the bait composition comprises at least 2, 3, 4, 5, 6, or 7 different overlapping peptides associated a mutation in a gene. In some embodiments, the gene is an oncogene.

In some embodiments, the peptides in the bait composition are associated with mutations in at least two, three, four, five or six genes. In some embodiments, the genes are oncogenes.

In some embodiments, the bait composition does not comprise a fluorochrome or fluorophore.

In some embodiments, the MHC molecules match with at least one, two, three, four, five or six HLA type of the individual from where the sample is obtained. For example, MHC I molecules comprising HLA-A*24: 02 and HLA-A*11: 01 is used when the individual has both HLA-A*24: 02 and HLA-A*11: 01. For another examples, MHC I molecules comprising at least one specific HLA-A molecule, one specific HLA-B molecule and one specific HLA-C molecule are used when the individual has the specific HLA-A, HLA-B, and HLA-C molecules. Patient-specific NGS data from WGS, WES, or RNA-seq can be used to predict HLA types with computational tools such as Optiptype and Polysolver (polymorphic loci resolver). See e.g., Szolet et al., Bioinformatics 30, 3310-3316, e.g., Shukla et al., Nat. Biotechnol. 33, 1152-1158. Reads can be selected from the NGS data that potentially derived from the HLA region and then they can be fully aligned to a full-length genomic library of all known HLA alleles. See e.g., Nucleic Acids Res. 41, D1222-D1227.

In some embodiments, the MHC molecule is coupled with a chaperon molecule prior to being complexed to the peptide. See e.g., Overall et al., Nat Commun. 2020 Apr. 20; 11 (1): 1909.

In some embodiments, a high throughput peptide-MHC (pMHC) tetramer library is constructed. See e.g., Overall et al., Nat Commun. 2020 Apr. 20; 11 (1): 1909.

In some embodiments, the bait composition comprises a plurality of different display moieties. Use of the plurality of different display moieties can facilitate or promote identification of heterogeneous antigenic specific immune cells (e.g., T cells). In some embodiments, multiple display moieties comprise at least 2, 3, or 4 kinds of MHC molecules. In some embodiments, multiple display moieties comprise at least two (e.g., 2, or 3, or 4) different kinds of MHC class I molecules, optionally the at least two (e.g., 2, 3, or 4) different kinds of MHC class I molecules are selected from the group consisting of HLA-A*24: 02, HLA-A*11: 01, HLA-A*02: 01, and HLA-A*03: 01. In some embodiments, multiple display moieties comprise HLA-A*24: 02, HLA-A*11: 01, HLA-A*02: 01, and HLA-A*03: 01.

In some embodiments, at least two of the plurality of different display moieties in the bait composition comprise different antigenic peptides. In some embodiments, each of the plurality of different display moieties in the bait composition comprises a different antigenic peptide. In some embodiments, the plurality of different display moieties comprise a number (e.g., about 10, 20, 30, 40, 50, 60, 70, 80, 90 or 100) of different antigenic peptides. In some embodiments, at least 30%, 40%, 50%, 60%, 70%, 80%, or 90% of antigenic peptides for a cancer (e.g., a specific cancer) in a neoantigen database are included in the number of different antigenic peptides. In some embodiments, at least 30%, 40%, 50%, 60%, 70%, 80%, or 90% of antigenic peptides for a specific kind of MHC molecule (e.g., HLA-A*24: 02, HLA-A*11: 01, HLA-A*02: 01, or HLA-A*03: 01) for a cancer (e.g., a specific cancer) in a neoantigen database are included in the number of different antigenic peptides.

In some embodiments, the plurality of different display moieties comprise at least one kind of MHC molecule that is HLA-A*24: 02, wherein HLA-A*24: 02 is complexed with at least two different antigenic peptides. In some embodiments, HLA-A*24: 02 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for a cancer (e.g., a specific cancer) in a neoantigen database are included in the number of different antigenic peptides. In some embodiments, the plurality of different display moieties further comprises HLA-A*11: 01, optionally wherein HLA-A*11: 01 is complexed with at least two different antigenic peptides. In some embodiments, HLA-A*11: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*11: 01 for a cancer (e.g., a specific cancer) in a neoantigen database are included in the number of different antigenic peptides. In some embodiments, the plurality of different display moieties further comprises HLA-A*02: 01, optionally wherein HLA-A*02: 01 is complexed with at least two different antigenic peptides. In some embodiments, HLA-A*02: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*02: 01 for a cancer (e.g., a specific cancer) in a neoantigen database are included in the number of different antigenic peptides. In some embodiments, the plurality of different display moieties further comprises HLA-A*03: 01, optionally wherein HLA-A*03: 01 is complexed with at least two different antigenic peptides. In some embodiments, HLA-A*03: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*03: 01 for a cancer (e.g., a specific cancer) in a neoantigen database are included in the number of different antigenic peptides.

In some embodiments, the plurality of different display moieties comprise at least one kind of MHC molecule that is HLA-A*11: 01, wherein HLA-A*11: 01 is complexed with at least two different antigenic peptides. In some embodiments, HLA-A*11: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*11: 01 for a cancer (e.g., a specific cancer) in a neoantigen database are included in the number of different antigenic peptides. In some embodiments, the plurality of different display moieties further comprises HLA-A*02: 01, optionally wherein HLA-A*02: 01 is complexed with at least two different antigenic peptides. In some embodiments, HLA-A*02: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*02: 01 for a cancer (e.g., a specific cancer) in a neoantigen database are included in the number of different antigenic peptides. In some embodiments, the plurality of different display moieties further comprises HLA-A*03: 01, optionally wherein HLA-A*03: 01 is complexed with at least two different antigenic peptides. In some embodiments, HLA-A*03: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*03: 01 for a cancer (e.g., a specific cancer) in a neoantigen database are included in the number of different antigenic peptides.

In some embodiments, the plurality of different display moieties comprise at least one kind of MHC molecule that is HLA-A*02: 01, wherein HLA-A*02: 01 is complexed with at least two different antigenic peptides. In some embodiments, HLA-A*02: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*02: 01 for a cancer (e.g., a specific cancer) in a neoantigen database are included in the number of different antigenic peptides. In some embodiments, the plurality of different display moieties further comprises HLA-A*03: 01, optionally wherein HLA-A*03: 01 is complexed with at least two different antigenic peptides. In some embodiments, HLA-A*03: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*03: 01 for a cancer (e.g., a specific cancer) in a neoantigen database are included in the number of different antigenic peptides.

In some embodiments, the plurality of different display moieties comprise at least one kind of MHC molecule that is HLA-A*03: 01, wherein HLA-A*03: 01 is complexed with at least two different antigenic peptides. In some embodiments, HLA-A*03: 01 is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for HLA-A*03: 01 for a cancer (e.g., a specific cancer) in a neoantigen database are included in the number of different antigenic peptides.

In some embodiments, the plurality of different display moieties comprise at least four kinds of MHC molecules, wherein the at least four kinds of MHC molecules comprise HLA-A*24: 02, HLA-A*11: 01, HLA-A*02: 01, and HLA-A*03: 01. In some embodiments, each kind of the MHC molecules (HLA-A*24: 02, HLA-A*11: 01, HLA-A*02: 01) are complexed with at least two different antigenic peptides. In some embodiments, each kind of the MHC molecule is complexed with a number (e.g., about 10, 20, 30, 40, 50, 60, 70, 80, 90 or 100) of antigenic peptides, optionally wherein at least 30%, 40%, 50%, 60%, 70%, 80%, or 90% antigenic peptides for the specific MHC molecule for a cancer (e.g., a specific cancer) in a neoantigen database are included in the number of different antigenic peptides.

In some embodiments, the method comprises separately contacting each of a plurality of different display moieties with a sample from the individual separately and separately isolating the immune cell associated with each of the different display moiety. For example, different display moieties may comprise display moieties with antigenic peptides associated with different antigens (e.g., different neoantigens, e.g., KRAS-12D, KRAS-12G, KRAS-12C).

In some embodiments, the method comprises contacting the plurality of different display moieties with a sample from the individual, and analyzing the pool of immune cells. For example, use of multi-color-labeled MHC tetramers for multiplex flow cytometry, pMHC tetramers labeled for mass cytometry analysis, and DNA-labeled tetramers designed for sequencing analysis have also been reported. See e.g., Andersen et al. Nat Protoc. 2012 Apr. 12; 7 (5): 891-902.

### Exemplary Considerations for Bait Composition Design

In some embodiments, the one or more neoantigenic peptides are selected through one or more steps as described below.

1. Determination of a Set of Peptides that Cover an Optimized Number of Tumor Subclones

In some embodiments, the one or more neoantigenic peptides comprise a truncal peptide. Truncal peptides described herein refer to those presented by all or most tumor subclones, is prioritized for inclusion into the bait composition.

If there are no truncal peptides predicted to be presented with high probability, or if the number of truncal peptides predicted to be presented with high probability is small enough that additional non-truncal peptides can be included in the display moiety, then further peptides can be prioritized by estimating the number and identity of tumor subclones and choosing peptides so as to maximize the number of tumor subclones covered.

### 2. Neoantigen Selection

Neoantigenic peptides can be selected via various methods or processes. In some embodiments, an integrated multi-dimensional model described below is applied that places candidate neoantigens in a space with at least one or more of the following axes and optimizes selection using an integrative approach. See e.g., WO2019050994A.
1. Probability of sequencing artifact (lower probability of artifact is typically preferred).
2. Probability of presentation (higher probability of presentation is typically preferred).
3. Gene expression (higher expression is typically preferred).
4. Coverage of HLA genes (larger number of HLA molecules involved in the presentation of a set of neoantigens is typically preferred).
5. Coverage of both MHC classes (covering both MHC-I and MHC-II is preferred). Exemplary bait compositions, display moieties and neoantigens (e.g., for analyzing immune cells from an individual for the presence of a lung cancer (e.g., NSCLC))

Exemplary bait compositions were determined using the methodology described herein.

TABLE 1
Exemplary neoantigenic peptides

| SEQ ID NO | HLA type | sequence |
|---|---|---|
| 1 | HLA-A*24:02 | VYCEEYYLF |
| 2 | HLA-A*02:01 | YQANVVWKV |
| 3 | HLA-A*03:01 | ALYFNSQWK |
| 4 | HLA-A*02:01 | KLLSFHSV |
| 5 | HLA-A*02:01 | YLNEAVFNFV |
| 6 | HLA-A*24:02 | FYMHEYPEGW |
| 7 | HLA-A*01:01 | EIDLPRELEY |
| 8 | HLA-A*02:01 | KQDGYDSV |
| 9 | HLA-A*11:01 | LLQHYLLYR |
| 10 | HLA-A*11:01 | TTARMRTMR |
| 11 | HLA-A*11:01 | HTFHLQDHH |
| 12 | HLA-A*11:01 | STTGATDLK |
| 13 | HLA-A*11:01 | MTFAETYPA |
| 14 | HLA-A*03:01 | NLLLIRGFK |
| 15 | HLA-A*11:01 | HGYFWFMGR |
| 16 | HLA-A*11:01 | FVLAALMEY |
| 17 | HLA-A*03:01 | RLRRLPVPR |
| 18 | HLA-A*11:01 | QSLVPAHPK |
| 19 | HLA-A*11:01 | RSFLSWDSR |
| 20 | HLA-A*03:01 | RIQGYIIEK |
| 21 | HLA-A*03:01 | ALNGMPLLK |
| 22 | HLA-A*11:01 | GTSESRETR |
| 23 | HLA-A*24:02 | IFSHPLYNI |
| 24 | HLA-A*11:01 | AAYLLFYQR |
| 25 | HLA-A*24:02 | SYVNILRAI |
| 26 | HLA-A*11:01 | RVEPVNYPK |
| 27 | HLA-A*24:02 | GYMGQQNEL |
| 28 | HLA-A*11:01 | RTQEARPPR |
| 29 | HLA-A*11:01 | RTASEDHPR |
| 30 | HLA-A*11:01 | VTIFVYDVK |
| 31 | HLA-A*11:01 | VVCIDAFLK |
| 32 | HLA-A*11:01 | STDQPVIPK |
| 33 | HLA-A*11:01 | SVAWPQDRR |
| 34 | HLA-A*11:01 | PTFGLSILK |
| 35 | HLA-A*24:02 | SYGLILLAF |
| 36 | HLA-A*24:02 | VFPLLFGTF |
| 37 | HLA-A*24:02 | YYLCLRHRL |
| 38 | HLA-A*11:01 | TSFPLDANK |
| 39 | HLA-A*11:01 | SLMVCNHDK |
| 40 | HLA-A*24:02 | SYIYILIII |
| 41 | HLA-A*11:01 | GVFRRCWEK |
| 42 | HLA-A*11:01 | RSAAIASEK |
| 43 | HLA-A*11:01 | SSTHPHFVR |
| 44 | HLA-A*11:01 | NVLEINFIK |
| 45 | HLA-A*11:01 | SMVPVMYQK |
| 46 | HLA-A*11:01 | SVYCIGQRR |
| 47 | HLA-A*11:01 | YQMLSFVHK |
| 48 | HLA-A*24:02 | LYSFISRFI |
| 49 | HLA-A*11:01 | FSFRSCNFK |
| 50 | HLA-A*11:01 | ISYAKYFPK |
| 51 | HLA-B*44:02 | AEYQDMHSY |
| 52 | HLA-B*44:02 | VEHINISQDW |
| 53 | HLA-B*07:02 | RGRMQTASL |
| 54 | HLA-C*06:02 | VRINTARPV |
| 55 | HLA-B*27:05 | RRSMLFARH |
| 56 | HLA-C*05:01 | KTDTGVHATL |

In some embodiments, the one or more neoantigenic peptides comprise one or more (e.g., at least 2, 5, 10, 15, 20, 25, 30, 35, 40, 45, or 50) sequences selected from the group consisting of SEQ ID NOs: 1-56.

In some embodiments, the one or more neoantigenic peptides comprise the amino acid sequences set forth in SEQ ID NOs: 1-5. In some embodiments, the one or more neoantigenic peptides further comprise one or more (e.g., at least 2, 5, 10, 15, 20, 25, 30, 35, or 40) sequences selected from the group consisting of SEQ ID NOs: 6-50 and/or one or more (e.g., 1, 2, 3, 4, 5 or 6) sequences selected from the group consisting of SEQ ID NOs: 51-56.

In some embodiments, the one or more neoantigenic peptides comprise one or more (e.g., at least 2, 5, 10, 15, 20, 25, 30, 35, or 40) sequences selected from the group consisting of SEQ ID NOs: 6-50. In some embodiments, the one or more neoantigenic peptides comprise the amino acid sequences set forth in SEQ ID NOs: 6-50. In some embodiments, the one or more neoantigenic peptides further comprise one or more (e.g., 1, 2, 3, 4, or 5) sequences selected from the group consisting of SEQ ID NOs: 1-5 and/or one or more (e.g., 1, 2, 3, 4, 5 or 6) sequences selected from the group consisting of SEQ ID NOs: 51-56.

In some embodiments, the one or more neoantigenic peptides comprise the amino acid sequences set forth in SEQ ID NOs: 51-56. In some embodiments, the one or more neoantigenic peptides further comprise one or more (e.g., at least 2, 5, 10, 15, 20, 25, 30, 35, or 40) sequences selected from the group consisting of SEQ ID NOs: 6-50 and/or one or more one or more (e.g., 1, 2, 3, 4, or 5) sequences selected from the group consisting of SEQ ID NOs: 1-5.

In some embodiments, the one or more (e.g., 1, 2, 3, 4, 5, 6, 7, 8, 9, or 10) neoantigenic peptides comprise the amino acid sequences selected from the group consisting of SEQ ID NOs: 1, 6, 23, 25, 27, 35-37, 40, and 48.

In some embodiments, the one or more (e.g., 5, 10, 15, 20, 25, or 30) neoantigenic peptides comprise the amino acid sequences selected from the group consisting of SEQ ID NOs: 9-13, 15, 16, 18, 19, 22, 24, 26, 28-34, 38, 39, 41-47, 49, and 50.

In some embodiments, the one or more (e.g., 1, 2, 3, or 4) neoantigenic peptides comprise the amino acid sequences selected from the group consisting of SEQ ID NOs: 2, 4, 5, and 8.

In some embodiments, the one or more (e.g., 1, 2, 3, 4, or 5) neoantigenic peptides comprise the amino acid sequences selected from the group consisting of SEQ ID NOs: 3, 14, 17, 20, and 21.

In some embodiments, the one or more (e.g., 1, 2, 3, or 4) neoantigenic peptides comprise the amino acid sequences selected from the group consisting of SEQ ID NOs: 53, 55, 51, and 52.

In some embodiments, the one or more (e.g., 1 or 2) neoantigenic peptides comprise the amino acid sequences selected from the group consisting of SEQ ID NOs: 54 and 56.

In some embodiments, the one or more neoantigenic peptides comprise amino acid sequences set forth in SEQ ID NOs: 1-56.

In some embodiments, the bait composition comprises one or more display moieties comprising one or more (e.g., at least 2, 5, 10, 15, 20, 25, 30, 35, 40, 45, or 50) MHC-peptide complexes (i.e., pMHC) paired as shown in Table 1. In some embodiments, the bait composition comprises one or more display moieties comprising pMHC comprising one or more neoantigenic peptides set forth in SEQ ID NOs 1-5 complexed with a MHC molecule according to Table 1. In some embodiments, the bait composition comprises one or more display moieties comprising pMHC comprising one or more (e.g., at least 2, 5, 10, 15, 20, 25, 30, 35, or 40) neoantigenic peptides comprising amino acids selected from the group consisting of SEQ ID NOs: 6-50 complexed with a MHC molecule according to Table 1. In some embodiments, the bait composition comprises one or more display moieties comprising pMHC comprising one or more (e.g., 1, 2, 3, 4, 5 or 6) neoantigenic peptides comprising amino acids selected from the group consisting of SEQ ID NOs: 51-56 complexed with a MHC molecule according to Table 1.

In some embodiments, the display moieties comprise one or more MHC-peptide complexes comprising neoantigenic peptides comprising one or more (e.g., 1, 2, 3, 4, 5, 6, 7, 8, 9, or 10) amino acid sequences selected from the group consisting of SEQ ID NOs: 1, 6, 23, 25, 27, 35-37, 40, and 48, wherein the MHC molecule is HLA-A*24: 02. In some embodiments, the display moieties comprise one or more MHC-peptide complexes comprising neoantigenic peptides comprising amino acid sequences set forth in SEQ ID NOs: 1, 6, 23, 25, 27, 35-37, 40, and 48, wherein the MHC molecule is HLA-A*24: 02.

In some embodiments, the display moieties comprise one or more MHC-peptide complexes comprising neoantigenic peptides comprising (e.g., 5, 10, 15, 20, 25, or 30) amino acid sequences selected from the group consisting of SEQ ID NOs: 9-13, 15, 16, 18, 19, 22, 24, 26, 28-34, 38, 39, 41-47, 49, and 50, wherein the MHC molecule is HLA-A*11: 01. In some embodiments, the display moieties comprise one or more MHC-peptide complexes comprising neoantigenic peptides comprising amino acid sequences set forth in SEQ ID NOs: 9-13, 15, 16, 18, 19, 22, 24, 26, 28-34, 38, 39, 41-47, 49, and 50, wherein the MHC molecule is HLA-A*11: 01.

In some embodiments, the display moieties comprise one or more MHC-peptide complexes comprising neoantigenic peptides comprising one or more (e.g., 1, 2, 3, or 4) amino acid sequences selected from the group consisting of SEQ ID NOs: 2, 4, 5, and 8, wherein the MHC molecule is HLA-A*02: 01. In some embodiments, the display moieties comprise one or more MHC-peptide complexes comprising neoantigenic peptides comprising amino acid sequences set forth in SEQ ID NOs: 2, 4, 5, and 8, wherein the MHC molecule is HLA-A*02: 01.

In some embodiments, the display moieties comprise one or more MHC-peptide complexes comprising neoantigenic peptides comprising one or more (e.g., 1, 2, 3, 4 or 5) amino acid sequences selected from the group consisting of SEQ ID NOs: 3, 14, 17, 20, and 21, wherein the MHC molecule is HLA-A*03: 01. In some embodiments, the display moieties comprise one or more MHC-peptide complexes comprising neoantigenic peptides comprising amino acid sequences set forth in SEQ ID NOs: 3, 14, 17, 20, and 21, wherein the MHC molecule is HLA-A*03: 01.

In some embodiments, the display moieties comprise one or more MHC-peptide complexes comprising neoantigenic peptides comprising one or more (e.g., 1, 2, 3, or 4) amino acid sequences selected from the group consisting of SEQ ID NOs: 53, 55, 51, and 52, wherein the MHC molecule is HLA-B molecule. In some embodiments, the display moieties comprise one or more MHC-peptide complexes comprising neoantigenic peptides comprising amino acid sequences set forth in SEQ ID NOs: 53, 55, 51, and 52, wherein the MHC molecule is HLA-B molecule.

In some embodiments, the display moieties comprise one or more MHC-peptide complexes comprising neoantigenic peptides comprising one or more amino acid sequences selected from the group consisting of SEQ ID NOs: 54 and 56, wherein the MHC molecule is HLA-C molecule. In some embodiments, the display moieties comprise one or more MHC-peptide complexes comprising neoantigenic peptides comprising amino acid sequences set forth in SEQ ID NOs: 54 and 56, wherein the MHC molecule is HLA-C molecule.

In some embodiments, the display moieties comprise MHC-peptide complexes comprising neoantigenic peptides comprising amino acid sequences set forth in SEQ ID NO: 1-56, wherein each of the neoantigen peptides is complexed with a MHC molecule according to Table 1.

### Modifications of Neoantigenic Peptides

Neoantigenic peptides having a desired activity or property can be modified to provide certain desired attributes, while increasing or at least retaining substantially all of the biological activity of the unmodified peptide to bind the desired MHC molecule and activate the appropriate immune cell (e.g., a T cell). For instance, neoantigenic peptide described herein can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gin; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232: 341-347 (1986), Barany &Merrifield, The Peptides, Gross &Meienhofer, eds. (N.Y., Academic Press), pp. 1-284 (1979); and Stewart & Young, Solid Phase Peptide Synthesis, (Rockford, 111, Pierce), 2d Ed. (1984).

Proteins or peptides described herein can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

Bait compositions comprising antigenic peptides associated with a pathogen or autoimmune disease can be similarly designed and/or selected.

Isolating, Culturing and Further Analyzing the Immune Cells that Bind to the Display Moiety

In some embodiments, the methods described herein comprise isolating an immune cell associated with the display moiety. In some embodiments, the methods comprise isolating immune cells prior to detecting immune cells binding to bait composition. In some embodiments, the methods comprise isolating immune cells following detecting immune cells binding to bait composition.

In some embodiments, the method does not comprise isolating immune cells prior to detecting immune cells binding to bait composition.

In some embodiments, the methods described herein comprise further analysis of the antigen-specific immune cells. In some embodiments, the method comprises demobilization of the immune cells or releasing the immune cells from the solid substrate for further analysis.

In some embodiments, the methods described herein further provides culturing the immune cell following the detecting immune cells binding to bait composition and/or prior to further analysis. In some embodiments, the methods described herein does not comprise a step of culturing the immune cell prior to further analysis.

In some embodiments, the isolating step comprises separating immune cells associated with the display moiety from the rest of the sample prior to detecting the immune cells associated with the display moiety.

In some embodiments, the isolating step (e.g., the isolating step prior to detecting immune cells associated with the display moiety) comprises using fluorescence-activated cell sorting (FACS). In some embodiments, the display moiety may be attached to one or more fluorescent attachment moieties, such as a streptavidin core attached or bound to a fluorescent molecule. In some embodiments, the display moiety is fluorescent or conjugated to a fluorophore directly. In some embodiments, multiple elements within the display moiety (such as the particle, attachment moiety, binding component) can be fluorescent, including each comprising a different fluorophore. In some embodiments, the display moiety used is magnetic or non-magnetic. In some embodiments, magnetic separation methods can be used in conjunction with FACS (e.g., before, after, or before and after FACS). In some embodiments, magnetic activated cell sorting, affinity chromatography, or any of the methods of cell sorting known in the art are used.

In some embodiments, the immune cell is selected from the group consisting of: a cytotoxic T cell (e.g., a CD8+ T cell), a helper T cell (e.g., a CD4+ T cell), a memory T cell, and a tumor infiltrating T cell. In some embodiments, the immune cell is a helper T cell.

In some embodiments, the immune cell is a B cell. In some embodiments, the B cell is a memory B cell.

In some embodiments, the immune cell is an isolated single immune cell. In some embodiments, the immune cell is in a mixture of immune cells. For example, the immune cell (e.g., a T cell) can be in a mixture of immune cells (e.g., a mixture containing both T cells and antigen presenting cells) when contacting with the display moiety. In some embodiments, the mixture of immune cells is a mixture comprising T cells, memory T cells, macrophage cells, or dendritic cells, or combinations thereof.

In some embodiments, culturing the immune cell comprises incubating the immune cell (such as a T cell) with one or more antigenic peptides described herein. In some embodiments, culturing the immune cell (such as a T cell) comprising incubating the immune cell (such as a T cell) with one or more antigenic peptides described herein for at least 1, 2, 3, 4, 5, 6, 7, 8, 9, 10, 11, 12, 13, 14, 15 days. In some embodiments, culturing the immune cell (such as a T cell) comprising incubating the immune cell (such as a T cell) with one or more antigenic peptides described herein for less than 5, 4, 3, 2, or 1 day. In some embodiments, culturing the immune cell comprises incubating the immune cell (such as a T cell) with one or more cytokines (e.g., IL-2, IL-7, IL-15).

In some embodiments, further analyzing the immune cell comprises quantifying the immune cell. In some embodiments, further analyzing the immune cell comprises sequencing one or more nucleic acids in the immune cell. In some embodiments, further analyzing the immune cell further comprises analyzing the sequences of the one or more nucleic acids. In some embodiments, the one or more nucleic acids comprises a nucleic acid sequence selected from the group consisting of a TCR sequence. In some embodiments, analyzing the sequences of the one or more nucleic acids comprises whole genome sequencing. In some embodiments, analyzing the sequences of the one or more nucleic acids comprises RNA sequencing. In some embodiments, analyzing the sequences of the one or more nucleic acids comprises single cell sequencing (such as scRNAseq). In some embodiments, analyzing the sequences of the one or more nucleic acids comprises decoding the barcode sequences. In some embodiments, analyzing the sequences of the one or more nucleic acids comprises identifying the rearranged genes on the tumor specific neoantigen activated immune cells. In some embodiments, analyzing the sequences of the one or more nucleic acids comprises identifying the rearranged T cell receptor (TCR) genes.

In some embodiments, further analyzing the immune cell comprises counting number or percentage of immune cells that bind to the bait composition and determining if the number or percentage of immune cells is above a threshold level, wherein the number or percentage of immune cells above a threshold level is indicative of cancer in the individual. In some embodiments, the immune cells are T cells (e.g., total T cells). In some embodiments, the immune cells are CD8+ T cells. In some embodiments, the immune cells are CD4+ T cells. In some embodiments, the immune cells are memory T cells. In some embodiments, the threshold is at least about 10, 20, 30, 40, 50, 75, 100, 150, 200, 250, 500, 1000, 2000, 3000, 4000, or 5000 immune cells that bind to the bait composition within a total of about 1×10⁵, 5×10⁵, 1×10⁶, 2×10⁶, 6×10⁶, 1×10⁷, 2×10⁷, 5×10⁷, or 1×10⁸ cells (e.g., immune cells) obtained in the sample (e.g., blood) from the individual. In some embodiments, at least about 1×10⁵ CD8 cells are analyzed, and cut-offs are set to more than 0.001% (such as more than about 0.001%, 0.002%, 0.003%, 0.004%, or 0.005% of CD8 cells and more than at least 2, 3, 4, 5, 6, 7, 8, 9, or 10 events. In some embodiments, at least about 1×10⁵ CD8 cells are analyzed, and cut-offs are set to more than 0.005% of CD8 cells and more than at least about 10 events.

Analysis of the immune repertoire usually involves one or more diversity indexes, such as Shannon entropy, clonality, and high-expanded clone (HEC) ratio, which are used to evaluate the amplification status of different TCR sequences and determine whether there is a high expansion of a few T-cell clones. See e.g., Li et al., Cancer Commun (Lond). 2020 October; 40 (10): 473-483. It has been found that the highest diversity was found in peripheral blood samples of the healthy population according to the calculation results of Shannon entropy. Moreover, TCR repertoire diversity in sentinel lymph nodes from patients with tumor was higher than in tumor tissues.

In some embodiments, analyzing immune cells comprises analyzing repertoire diversity (e.g., Shannon diversity) of T cells obtained from the individual. In some embodiments, analyzing repertoire diversity comprises comparing the repertoire diversity of T cells obtained from the individual to that of T cells from a reference individual (such as a healthy individual, or a group of healthy individuals) and determine if the repertoire diversity is below a threshold, wherein the repertoire diversity (e.g., Shannon diversity) being below a threshold diversity is indicative of cancer in the individual. In some embodiments, the threshold is a Shannon diversity that is lower (e.g., at least about 5%, 10%, 15%, 20%, 25% or 30%) lower than that of T cells from a reference individual, it is indicative of cancer in the individual.

In some embodiments, further analyzing the immune cell comprises generating a signature profile. In some embodiments, the signature profile is associated with a specific cancer. In some embodiments, the cancer is hepatocellular carcinoma (HCC), and the signature profile comprises a three gene signature, wherein the three genes are CXCR2, CCR2 and EP400. See e.g., Shi et al., Eur J Cancer. 2014 March; 50 (5): 928-36.

In some embodiments, further analyzing the immune cell comprises generating a CD8 and/or CD4 T cell signature profile (e.g., gene expression profile (RNA-seq), gene rearrangement, 5mC, 5hmC or 5caC profile).

In some embodiments, further analyzing the immune cell comprises sequencing one or more nucleic acids and generating a library. In some embodiments, the one or more nucleic acids are repertoire related nucleic acids (e.g., VDJ gene-related DNA or RNA). In some embodiments, analyzing the isolating immune cells further comprises analyzing repertoire of the T cells as described above.

In some embodiments, analyzing the sequences of the one or more nucleic acids comprises a) obtaining an enriched sample from the immune cell, wherein the enriched sample is enriched for the one or more nucleic acids and b) sequencing the one or more nucleic acids in the enriched sample.

In some embodiments, further analyzing the immune cell further comprises subjecting the immune cell to mass spectrometry analysis. In some embodiments, further analyzing the immune cell further comprises subjecting the immune cell to Assay for Transposase-Accessible Chromatin using sequencing (ATAC)-sequencing. In some embodiments, further analyzing the immune cell further comprises subjecting the immune cell to chromatin immunoprecipitation (ChIP)-sequencing.

Epigenetic modifications of tumor specific immune cells (e.g., T cells, e.g., CD8+ T cells) have been reported. See e.g., Yang et al., Genome Biol 21, 2 (2020); Villanueva et al., Trends Immunol. 2020 August; 41 (8): 676-691. In some embodiments, further analyzing the immune cell further comprises identifying one or more epigenetic modifications in the T cell receptor genes of the immune cell. In some embodiments, the one or more epigenetic modifications comprises histone acetylation, histone ubiquitination, and/or histone methylation. In some embodiments, the one or more epigenetic modifications comprises DNA or RNA methylation, hydroxylation and/or histone glycosylation.

### Individual

In some embodiments, the individual described herein is a mammal (such as a human, dog, cat, or horse). In some embodiments, the individual is a human.

In some embodiments, the individual has not previously been diagnosed as having a cancer (e.g. any cancer or a specific type of cancer).

In some embodiments, the individual has previously been diagnosed as having a cancer.

In some embodiments, the individual has minimal residual disease (MRD). In some embodiments, the individual has minimal residual cancer. In some embodiments, the minimal residual cancer is seen after the cancer was surgical resected or cured. In some embodiments, the minimal residual disease is too minimal to be detected by imaging instruments (e.g., a routinely used or standard imaging instrument used for detection of the cancer). In some embodiments, the location of the minimal residual disease is diverse. In some embodiments, the minimal residual cancer is a result of immune escape or resistance to treatment. In some embodiments, the individual has been previously treated for cancer and exhibits no pathological symptom of a cancer after the treatment.

In some embodiments, the individual is at risk of developing cancer. In some embodiments, the risk of having cancer is based on any one or more factors selected from the group consisting of family history, mutations, environmental factors, and age. In some embodiments, the individual is predicted by a doctor to have a risk of at least 20%, 30%, 40%, or 50% to develop a cancer (such as any specific kind of cancer) based upon any one or more factors selected from the group consisting of family history, mutations, environmental factors, and age.

In some embodiments, the individual is a human and is at least about 50 years old (e.g., such as at least about 50, 60, 70, or 80 years old).

In some embodiments, the individual is no more than 14 years old.

In some embodiments, the individual is a male. In some embodiments, the individual is a female.

### Cancer

The cancer described herein can be a cancer in general, or any type of cancer. In some embodiments, the cancer is a solid tumor. In some embodiments, the cancer is a liquid cancer.

In some embodiments, the cancer is a carcinoma, a sarcoma, a myeloma, a leukemia, a lymphoma, a blastoma, a germ cell tumor, or any combination thereof. In some embodiments, the cancer is a squamous cell carcinoma or an adenocarcinoma.

In some embodiments, the cancer is selected from the group consisting of: small cell lung cancer, non-small-cell lung cancer, nasopharyngeal cancer, colorectal cancer, anal cancer, liver cancer, bladder cancer, testicular cancer, cervical cancer, ovarian cancer, gastric cancer, esophageal cancer, head-and-neck cancer, pancreatic cancer, prostate cancer, renal cancer, thyroid cancer, melanoma cancer, and breast cancer.

In some embodiments, the cancer is a recurrent cancer.

### Generating Report

In some embodiments, the method of analyzing a sample of an individual further comprises generating a report comprising information about the disease or condition status (such as cancer status) in the individual.

In some embodiments, the information about disease or condition (e.g., cancer) status comprises likelihood of the presence of a disease or condition (e.g., cancer), classification of disease or condition (e.g., cancer), type of disease or condition (e.g., cancer), nature of disease or condition (e.g., cancer), origin of disease or condition (e.g., cancer), stage of disease or condition (e.g., cancer), likelihood of disease or condition (e.g., cancer) progression, likelihood of developing one or more disease or condition (e.g., cancer) symptoms, and/or treatment options for the individual.

The information about the disease or condition (e.g., cancer) status is based upon the results gathered from analyzing the immune cells that bind to the bait composition as discussed above. In some embodiments, the likelihood of the presence of a disease or condition (e.g., cancer) is wholly or at least partly based upon the number and/or percentage of immune cells (e.g., T cells) that bind to the bait composition. For example, when the number and/or percentage of immune cells (e.g., T cells, e.g., CD8 T cells, e.g., CD4 T cells) that bind to the bait composition is significantly above a threshold level (such as a threshold level described above), the generated report comprises the information that the disease or condition (e.g., cancer) is present in the individual. When the number and/or percentage of immune cells (e.g., T cells, e.g., CD8 T cells, e.g., CD4 T cells) is near or above threshold but not significantly above threshold, the generated report comprises the information that there is a median or high likelihood of the presence of the disease or condition (e.g., cancer).

In some embodiments, the likelihood of the presence of a disease or condition (e.g., cancer) is wholly or at least partly based upon the repertoire diversity of T cells that bind to the bait composition. For example, when the repertoire diversity (e.g., Shannon diversity) of the T cells (e.g., CD8 T cells, e.g., CD4 T cells) that bind to the bait composition is significantly below the threshold, the generated report comprises the information that the disease or condition (e.g., cancer) is present in the individual. When the repertoire diversity (e.g., Shannon diversity) of the T cells (e.g., CD8 T cells, e.g., CD4 T cells) that bind to the bait composition is near or below the threshold but not significantly below the threshold, the generated report comprises the information that there is a median or high likelihood of the presence of the disease or condition (e.g., cancer).

In some embodiments, the information about disease or condition (e.g., cancer) status comprises gene expression profile signature, a gene mutation profile signature, and/or an epigenetic modification signature. In some embodiments, the signature epigenetic modification comprises a DNA methylation signature and a histone glycosylation signature.

### EXEMPLARY EMBODIMENTS

Embodiment 1. A method of analyzing a sample from one or more individuals for the presence or absence of immune cells capable of binding to an antigenic peptide, the method comprising:
a) immobilizing immune cells from the sample on a solid support, wherein the immune cells: 1) prior to immobilization, have been contacted with a bait composition comprising a display moiety comprising the antigenic peptide under a condition sufficient for an immune cell to bind to the display moiety; or 2) after the immobilization, are contacted with a bait composition comprising a display moiety comprising the antigenic peptide under a condition sufficient for an immune cell to bind to the display moiety;
b) contacting the immobilized immune cells with a detection agent recognizing the display moiety under a condition sufficient for the agent to bind to the display moiety; and
c) detecting the detection agent, thereby detecting the immune cells bound to the display moiety,
optionally wherein the method further comprises isolating immune cells bound to the display moiety or removing the display moiety or immune cells not bound to each other prior to step c).

Embodiment 2. A method of analyzing a sample from one or more individuals for the presence or absence of immune cells capable of binding to an antigenic peptide, the method comprising:
a) contacting the immune cells with a bait composition comprising a display moiety comprising the antigenic peptide under a condition sufficient for an immune cell to bind to the display moiety;
b) contacting the immune cells with a detection agent recognizing the display moiety under a condition sufficient for the agent to bind to the display moiety;
c) immobilizing the immune cells from the sample on a solid support, and
d) detecting the detection agent, thereby detecting the immune cells bound to the display moiety,
optionally wherein the method further comprises isolating immune cells bound to the display moiety or removing the display moiety or immune cells not bound to each other prior to step d).

Embodiment 3. The method of embodiment 1 or embodiment 2, wherein the sample is obtained from a single individual.

Embodiment 4. The method of embodiment 1 or embodiment 2, wherein the sample is a mixture a plurality of individual samples, each obtained from a different individual.

Embodiment 5. The method of embodiment 4, wherein prior to being combined to form the mixture each of the plurality of individual samples is separately processed such that the immune cells contained therein are associated with a unique sample barcode (e.g., via mixing the immune cells with a molecule comprising the unique sample barcode). In some embodiments, the method further comprises removing unbound molecule comprising the barcode (molecules not bound by the immune cells) e.g., via washing the immune cells for at least one time.

Embodiment 6. The method of embodiment 5, wherein the method further comprises determining the unique barcode associated with the immune cells bound to the display moiety, thereby identifying the sample from which the immune cells are derived.

Embodiment 7. The method of any one of embodiments 1 and 3-6, wherein the immune cells have been contacted with a bait composition comprising a display moiety comprising an antigenic peptide under a condition sufficient for an immune cell to bind to the display moiety prior to the immobilization.

Embodiment 8. The method of any one of embodiments 1 and 3-6, wherein the immune cells are contacted with a bait composition comprising a display moiety comprising an antigenic peptide under a condition sufficient for an immune cell to bind to the display moiety following the immobilization.

Embodiment 9. A method of analyzing a plurality of samples from one or more individuals for the presence or absence of immune cells capable of binding to an antigenic peptide, comprising:
a) separately processing each of the plurality of individual samples such that immune cells contained therein are associated with a unique sample barcode,
b) combining the plurality of processed samples to form a sample mixture,
c) contacting the sample mixture with a bait composition comprising a display moiety comprising an antigenic peptide under a condition sufficient for an immune cell to bind to the display moiety, thereby forming a bait-sample mixture,
d) detecting immune cells bound to the display moiety in the bait-sample mixture; and
e) determining the unique barcode associated with the immune cells bound to the display moiety, thereby identifying the sample from which the immune cells are derived, optionally wherein the method further comprises isolating immune cells bound to the display moiety or removing the display moiety or immune cells not bound to each other prior to step d).

Embodiment 10. The method of any one of embodiments 1-9, wherein the immune cells comprise T cells, optionally the immune cells comprise no more than about 10%, 5%, 4%, 3%, 2% or 1% T cells specifically binding to antigenic peptide in the bait composition.

Embodiment 11. The method of embodiment 10, wherein the immune cells are selected from the group consisting of: a cytotoxic T cell, a memory T cell, and a tumor infiltrating T cell.

Embodiment 12. The method of any one of embodiments 1-9, wherein the immune cells comprise B cells.

Embodiment 13. The method of any one of embodiments 1-12, wherein the method further comprises removing unbound display moiety following contacting the immune cells with the agent recognizing the display moiety and prior to detecting the immune cells bound to the display moiety.

Embodiment 14. The method of any one of embodiments 1-13, wherein the antigenic peptide is associated with a cancer or tumor antigen, optionally wherein the antigenic peptide is a neoantigen peptide.

Embodiment 15. The method of embodiment 14, wherein the sample is obtained from one or more individuals exhibiting no pathological symptom of a cancer.

Embodiment 16. The method of any one of embodiments 1-13, wherein the antigenic peptide is associated with a pathogen antigen, optionally wherein the antigenic peptide is associated with a virus, a bacteria, or a fungus antigen.

Embodiment 17. The method of any one of embodiments 1-13, wherein the antigenic is an autoantigenic peptide associated with an autoantigen.

Embodiment 18. The method of any one of embodiments 1-8 and 10-17, wherein the immune cells are immobilized on the solid substrate such as a bead.

Embodiment 19. The method of embodiment 18, wherein the bead is associated with an antibody recognizing the immune cells, optionally wherein the antibody is a CD8 antibody.

Embodiment 20. The method of any one of embodiments 1-19, wherein the display moiety comprises two or more antigenic peptides, optionally wherein the display moiety comprises four antigenic peptides.

Embodiment 21. The method of embodiment 20, wherein the two or more antigenic peptides in the display moiety are the same.

Embodiment 22. The method of embodiments 14-15 and 18-21, wherein the display moiety comprises a library of antigenic peptides for a mutation associated with a cancer or tumor, wherein the library comprises at least about 5, 10, 12, 15, 18 or 20 distinct neoantigenic peptides.

Embodiment 23. The method of any one of embodiments 1-22, wherein the antigenic peptide has one or more of the following characteristics:
a) having a binding affinity of about 1 nM to about 5000 nM to an MHC molecule;
b) having a binding affinity of about 1 nM to about 5000 nM to a cognate TCR molecule;
c) is hydrophobic; and
d) has high content of aromatic residues.

Embodiment 24. The method of any one of embodiments 14-15 and 18-23, wherein the antigenic peptide is a neoantigenic peptide comprising a mutation relative to a wildtype peptide, optionally at the third amino acid position counting from the N-terminus.

Embodiment 25. The method of any one of embodiments 1-24, wherein the antigenic peptide has low immunogenicity.

Embodiment 26. The method of any one of embodiments 1-25, wherein the display moiety comprises an MHC molecule complexed with the antigenic peptide, optionally wherein the MHC molecule comprises a MHC multimer, further optionally the MHC multimer is a MHC tetramer or a MHC dextramer.

Embodiment 27. The method of embodiment 26, wherein the MHC molecule comprises a MHC class I molecule.

Embodiment 28. The method of embodiment 27, wherein the MHC class I molecule is selected from the group consisting of HLA-A, HLA-B, and HLA-C, optionally wherein MHC class I molecule comprises a HLA-A molecule, further optionally wherein the HLA-A molecule comprises a mutation that reduces its binding to CD8, further optionally where the HLA-A molecule comprises a HLA A2 heavy chain with a A245V mutation.

Embodiment 29. The method of embodiment 27 or 28, wherein the antigenic peptide is about 8 to about 10 amino acids long.

Embodiment 30. The method of embodiment 26, wherein the MHC comprises a MHC class II molecule.

Embodiment 31. The method of embodiment 30, wherein the MHC class II molecule is selected from the group consisting of HLA-DQ and HLA-DR.

Embodiment 32. The method of embodiment 30 or 31, wherein the antigenic peptide is about 10 to about 20 amino acids long.

Embodiment 33. The method of any one of embodiments 26-32, wherein the display moiety comprises two or more different kinds of MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03.

Embodiment 34. The method of any one of embodiments 1-33, wherein the display moiety comprises a particle.

Embodiment 35. The method of embodiment 34, wherein the particle in the display moiety is selected from the group consisting of: a surface, a nanoparticle, a bead, and a polymer.

Embodiment 36. The method of embodiment 34 or 35, wherein the particle in the display moiety is a dextran particle.

Embodiment 37. The method of embodiment 34 or 35, wherein the particle in the display moiety is a magnetic nanoparticle or polystyrene nanoparticle.

Embodiment 38. The method of embodiment 34 or 35, wherein the particle in the display moiety is an agarose bead or a sepharose bead.

Embodiment 39. The method of any one of embodiments 34-38, wherein the antigenic peptide or MHC is directly attached to the particle in the display moiety.

Embodiment 40. The method of any one of embodiments 34-38, wherein the antigenic peptide or MHC is attached to the particle in the display moiety via a binding pair comprising a first binding component attached to the antigenic peptide and a second binding component bound to the particle.

Embodiment 41. The method of any one of embodiments 1-40, wherein the display moiety comprises a cell.

Embodiment 42. The method of embodiment 41, wherein the cell comprises a polynucleotide encoding the antigenic peptide.

Embodiment 43. The method of embodiment 42, wherein the polynucleotide encodes a plurality of antigenic peptides.

Embodiment 44. The method of any one of embodiments 1-43, wherein the display moiety further comprises a detectable label.

Embodiment 45. The method of embodiment 44, wherein the detectable label is a fluorophore.

Embodiment 46. The method of any one of embodiments 1-45, wherein the method further comprises isolating immune cells bound to the display moiety or removing the display moiety or immune cells not bound to each other.

Embodiment 47. The method of any one of embodiments 1-46, wherein the method further comprises phenotyping immune cells bound to the display moiety.

Embodiment 48. The method of any one of embodiments 1-47, wherein the method further comprises sequencing one or more nucleic acids in immune cells bound to the display moiety, optionally wherein the method further comprises analyzing the sequences of the one or more nucleic acids, optionally wherein the one or more nucleic acids is a TCR, CD8, CD45 sequences or any specific sequence that is characteristic of the captured immune cells, optionally wherein analyzing the sequences of the one or more nucleic acids comprises whole genome sequencing and/or RNAseq sequencing, alternatively, by a array of primer sequence-specific q-PCR, and/or RT-PCR.

Embodiment 49. The method of any one of embodiments 1-48, wherein the method further comprises subjecting the immune cell bound to the display moiety to mass spectrometry analysis.

Embodiment 50. The method of any one of embodiments 1-49, wherein the method further comprises identifying one or more epigenetic modifications in the immune cell, optionally wherein the more or more epigenetic modifications comprises DNA methylation and/or histone glycosylation.

Embodiment 51. The method of any one of embodiments 1-50, wherein the individual is a human.

Embodiment 52. The method of embodiment 51, wherein the individual is at least about 50 years old.

Embodiment 53. The method of any one of embodiments 14-15 and 18-52, wherein the individual a) has not previously been diagnosed as having a cancer, optionally wherein the individual is at risk of having cancer, or b) the individual has been previously treated for cancer and exhibits no pathological symptom of a cancer after the treatment.

Embodiment 54. The method of any one of embodiments 16 and 18-54, wherein the individual a) has not been diagnosed as having an infection by a pathogen, optionally wherein the individual is at risk of having the infection, and/or b) exhibits no pathological symptom of an infection by a pathogen, optionally exhibits no pathological symptom of an infection by the pathogen.

Embodiment 55. The method of any one of embodiments 17 and 18-54, wherein the individual a) has not been diagnosed as having an immune response against the autoantigen, optionally wherein the individual is at risk of developing the immune response against the autoantigen, and/or b) exhibits no pathological symptom of an immune response (e.g., an autoimmune disease or disorder), optionally exhibits no pathological symptom of an immune response against the autoantigen.

Embodiment 56. The method of any one of embodiments 1-55, wherein the sample is selected from the group consisting of: blood, plasma, and a peripheral blood mononuclear cell (PBMC) sample.

Embodiment 57. The method of any one of embodiments 1-57, wherein the bait composition comprises a plurality of different display moieties.

Embodiment 58. The method of embodiment 57, wherein the each of the plurality of different display moieties in the bait composition comprises a different antigenic peptide.

Embodiment 59. The method of embodiment 57 or 58, wherein the plurality of different display moieties in the bait composition comprises at least two different display moieties, each comprising a different MHC molecule.

Embodiment 60. The method of embodiment 59, wherein the plurality of different display moieties in the bait composition comprises at least four different display moieties, each comprising a different MHC molecule.

Embodiment 61. The method of embodiment 60, wherein the plurality of different display moieties in the bait composition comprises at least 100 different display moieties, each comprising a different MHC molecule.

Embodiment 62. The method of any one of embodiments 59-61, wherein each of the different display moieties comprising different MHC molecules comprises a different detectable label.

Embodiment 63. The method of embodiment 62, wherein the different detectable labels comprise fluorophore(s).

Embodiment 64. The method of any one of embodiments 1-6 and 10-63, wherein the agent comprises an antibody that binds to the display moiety.

Embodiment 65. A method of detecting antigen-specific immune cells in an individual, comprising: analyzing a sample from the individual according to any one of embodiments 1-64, wherein a predetermined characteristic of the immune cells is indicative of a disease or a disorder in the individual.

Embodiment 66. The method of embodiment 65, wherein the predetermined characteristic of the immune cell comprises the presence of the immune cells.

Embodiment 67. The method of embodiment 66, wherein the presence of immune cells specifically binds to a bait composition comprising one or more neoantigens is indicative of the presence of a cancer.

Embodiment 68. The method of embodiment 66, wherein the presence of immune cells specifically binds to a bait composition comprising one or more antigens of a pathogen (e.g., virus, bacteria, fungus) is indicative of the presence of an infection of the pathogen.

Embodiment 69. The method of embodiment 66, wherein the presence of immune cells specifically binds to a bait composition comprising one or more autoantigens associated with an autoimmune disease or disorder is indicative of the presence of the immune disease or disorder.

Embodiment 70. The method of any one of embodiments 66-69, wherein the predetermined characteristic of the immune cell comprises a quantity of the immune cell above a threshold level.

Embodiment 71. The method of any one of embodiments 65-70, wherein the predetermined characteristic of the immune cell comprises a gene expression profile signature, a gene mutation profile signature, and/or an epigenetic modification signature.

Embodiment 72. The method of embodiment 71, wherein the signature epigenetic modification comprises a DNA or RNA methylation, hydroxylation signature and a histone acetylation, methylation and/or glycosylation signature.

Embodiment 73. A method of detecting residual cancer in an individual, wherein the individual has been previously treated with an anti-cancer therapy and exhibits no pathological symptom of cancer after treatment, the method comprising analyzing a post-treatment sample from the individual according to the method of any one of embodiments 14-15, 18-53 and 56-64, wherein a predetermined characteristic of the immune cell from the post-treatment sample is indicative of residual cancer in the individual.

Embodiment 74. The method of embodiment 73, wherein the method comprises: a) analyzing a pre-treatment sample from the individual prior to anti-cancer therapy and a post-treatment sample from the individual according to the method of any one of embodiments 14-15, 18-53 and 56-64, and b) comparing the characteristics of the immune cells from the pre-treatment sample and immune cells from the post-treatment sample; wherein a predetermined difference in characteristics of the immune cell from the pre-treatment sample and the immune cell from the post-treatment sample is indicative of residual cancer in the individual.

Embodiment 75. A method of treating a cancer in an individual, comprising a) diagnosing the individual as having cancer according to the method of any one of embodiments 67 and 70-72; and b) subjecting the individual to an anti-cancer therapy.

Embodiment 76. The method of embodiment 75, wherein the anti-cancer therapy is not an immunotherapy.

Embodiment 77. The method of any one of embodiments 1-76, wherein the cancer is a solid tumor.

Embodiment 78. A method of treating an infection in an individual, comprising a) diagnosing the individual as having an infection by a pathogen according to the method of any one of embodiments 68 and 70-72; and b) subjecting the individual to a therapy for the pathogen.

Embodiment 79. A method of treating an autoimmune disease or disorder in an individual, comprising a) diagnosing the individual as having an autoimmune disease or disorder according to the method of any one of embodiments 69-72; and b) subjecting the individual to a therapy for the disease or disorder.

Embodiment 80. A display moiety comprising a particle associated with a plurality of MHC-peptide complexes, wherein the plurality of MHC-peptide complexes comprises no less than or more than 2, 3, 4, 5, 6, 8, 9, 10, or 12 MHC-peptide complexes, wherein each of the MHC-peptide complex comprises at least one MHC molecule complexed with a peptide, wherein at least two or more of the MHC-peptide complexes are different.

Embodiment 81. The display moiety of embodiment 80, wherein one or more of the plurality of MHC-peptide complexes is an MHC-peptide monomer, an MHC-peptide dimer, an MHC-peptide trimer, an MHC-peptide tetramer, an MHC-peptide pentamer, an MHC-peptide octamer, an MHC-peptide dextramer, or an MHC-peptide dodecamer, optionally wherein each of the plurality of MHC-peptide complex is an MHC-peptide monomer, an MHC-peptide dimer or an MHC-peptide trimer. In some embodiments, at least one MHC-peptide complex in the plurality of MHC-peptide complexes has two distinct MHC-peptide monomers. In some embodiments, the at least one MHC complex is an MHC-peptide dimer, an MHC-peptide trimer, or an MHC-peptide tetramer.

Embodiment 82. The display moiety of embodiment 80 or embodiment 81, wherein the plurality of MHC-peptide complexes comprises at least 20 MHC-peptide complexes, optionally wherein the plurality of MHC-peptide complexes comprise at least 50 or 100 MHC-peptide complexes.

Embodiment 83. The display moiety of any one of embodiments 80-82, wherein the MHC-peptide complex is attached to the particle via a binding pair comprising a first binding component attached to MHC-peptide complex or a unit thereof (i.e., a MHC-peptide monomer) and a second binding component bound to the particle.

Embodiment 84. The display moiety of embodiment 83, wherein the first binding component comprises a biotin, and the second binding component comprises streptavidin.

Embodiment 85. The display moiety of any one of embodiments 80-84, wherein the particle is solid.

Embodiment 86. The display moiety of any one of embodiments 80-85, wherein the particle has a diameter of about 0.1 μm to about 150 μm or 0.1 μm to about 50 μm (e.g., at least about 0.1 μm, 0.2 μm, 0.25 μm, 0.5 μm, 0.75 μm, or 1 μm).

Embodiment 87. The display moiety of embodiment 86, wherein the particle has a diameter of about 0.5 μm to about 20 μm, optionally wherein the particle has a diameter of about 0.5 μm to about 10 μm, further optionally wherein the particle has a diameter of about 1 μm.

Embodiment 88. The display moiety of any one of embodiments 80-87, wherein the particle has a surface area of about 1 μm² to about 20 μm².

Embodiment 89. The display moiety of any one of embodiments 80-88, wherein the particle has a number of about 10 to about 10000 second binding component associated with it.

Embodiment 90. The display moiety of any one of embodiments 80-89, wherein the particle is spherical, optionally wherein the particle is a bead.

Embodiment 91. The display moiety of any one of embodiments 80-90, wherein the particle is magnetic.

Embodiment 92. The display moiety of any one of embodiments 80-91, wherein display moiety comprises at least two different MHC molecules.

Embodiment 93. The display moiety of embodiment 92, wherein the at least two different MHC molecules comprises two different MHC class I molecules.

Embodiment 94. The display moiety of embodiment 92 or embodiment 93, wherein the at least two different MHC molecules comprises two different MHC class II molecules.

Embodiment 95. The display moiety of any one of embodiments 92-94, wherein the at least two different MHC molecules comprises both an MHC class I molecule and an MHC class II molecule.

Embodiment 96. The display moiety of any one of embodiments 80-95, wherein the display moiety comprises an HLA-A molecule, further optionally wherein the HLA-A molecule comprises a mutation that reduces its binding to CD8, further optionally where the HLA-A molecule comprises an HLA A2 heavy chain with a A245V mutation.

Embodiment 97. The display moiety of any one of embodiments 80-96, wherein the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03.

Embodiment 98. The display moiety of any one of embodiments 80-97, wherein the display moiety comprises an MHC class II molecule selected from the group consisting of HLA-DQ and HLA-DR.

Embodiment 99. The display moiety of any one of embodiments 80-98, wherein the peptide is associated with a cancer or tumor antigen, optionally wherein the peptide is associated with a neoantigen.

Embodiment 100. The display moiety of any one of embodiments 80-99, wherein the peptide is associated with an antigen of a pathogen, optionally wherein the pathogen is a virus, bacteria of fungus.

Embodiment 101. The display moiety of any one of embodiments 80-100, wherein the peptide is associated with an autoantigen.

Embodiment 102. The display moiety of any one of embodiments 80-101, wherein the display moiety comprises at least two distinct peptides.

Embodiment 103. The display moiety of embodiment 102, wherein the display moiety comprises at least five distinct peptides, optionally wherein the display moiety comprises at least 10 distinct peptides.

Embodiment 104. The display moiety of embodiment 102 or embodiment 103, wherein at least two distinct peptides in the display moiety do not overlap.

Embodiment 105. The display moiety of any one of embodiments 102-104, wherein at least two distinct peptides in the display moiety overlaps.

Embodiment 106. The display moiety of any one of embodiments 102-105 wherein the at least two distinct peptides are associated with two different antigens.

Embodiment 107. The display moiety of any one of embodiments 102-106, wherein the two distinct antigens comprise two distinct mutations of one or more genes associated with a disease, optionally 1) wherein the disease is a cancer, the gene is a oncogene, or 2) wherein the disease is not a cancer or tumor.

Embodiment 108. The display moiety of any one of embodiments 80-107, wherein the display moiety does not comprise a fluorochrome or fluorophore.

Embodiment 109. A bait composition comprising the display moiety of any one of embodiments 80-108.

Embodiment 110. The bait composition of embodiment 109, wherein the bait composition comprises at least two different display moieties.

Embodiment 111. The bait composition of embodiment 109 or embodiment 110, wherein the bait composition comprises an HLA-A molecule, an HLA-B molecule, and an HLA-C molecule.

Embodiment 112. The bait composition of any one of embodiments 109-111, wherein the bait composition comprises HLA-A*24: 02, HLA-A*11: 01, HLA-A*02: 01, and HLA-A*03: 01.

Embodiment 113. The bait composition of any one of embodiments 109-112, wherein the bait composition comprises an HLA-DP molecule, an HLA-DQ molecule, and an HLA-DR molecule.

Embodiment 114. The bait composition of any one of embodiments 109-113, wherein the bait composition comprises at least 25, 50, 100, 150, 200, or 250 different peptides.

Embodiment 115. The bait composition of any one of embodiments 109-114, wherein the peptides in the bait composition are associated with at least 25, 50, 75, 100, 125, 150, 175, 200, 225, or 250 different antigens.

Embodiment 116. The bait composition of any one of embodiments 109-115, wherein the bait composition comprises at least 2, 3, 4, 5, 6, or 7 different overlapping peptides associated a mutation in a gene, optionally wherein the gene is an oncogene.

Embodiment 117. The bait composition of any one of embodiments 109-116, wherein the peptides in the bait composition are associated with mutations in at least two, three, four, five or six genes, optionally wherein the genes are oncogenes.

Embodiment 118. The bait composition of any one of embodiments 109-117, wherein the bait composition does not comprise a fluorochrome or fluorophore.

Embodiment 119. A method of analyzing immune cells in a sample from an individual, comprising a) contacting a plurality of immune cells with the display moiety of any one of embodiments 80-108 or a bait composition of any one of embodiments 109-118, b) detecting or analyzing the one or more immune cells that bind to one or more of the display moieties.

Embodiment 120. The method of embodiment 119, wherein the immune cells comprise T cells.

Embodiment 121. The method of embodiment 119 or embodiment 120, wherein the immune cells are selected from the group consisting of: cytotoxic T cells, memory T cells, and tumor infiltrating T cells, optionally wherein the T cells are CD4+ T cells and/or CD8+ T cells.

Embodiment 122. The method of any one of embodiments 119-121, wherein the immune cells comprise B cells.

Embodiment 123. The method of any one of embodiments 119-122, wherein the immune cells comprise a mixture of immune cells comprising T cells, B cells, macrophage cells, or dendritic cells, or combinations thereof.

Embodiment 124. The method of any one of embodiments 119-123, wherein the sample is selected from the group consisting of: blood, plasma, and a peripheral blood mononuclear cell (PMBC) sample.

Embodiment 125. The method of any one of embodiments 119-124, wherein the sample is obtained from a diseased tissue, optionally wherein the diseased tissue is a cancer tissue or tumor tissue.

Embodiment 126. The method of any one of embodiments 119-125, wherein the sample is obtained from a single individual.

Embodiment 127. The method of any one of embodiments 119-125, wherein the sample is a mixture a plurality of individual samples, each obtained from a different individual.

Embodiment 128. The method of any one of embodiments 119-127, wherein the individual is a human.

Embodiment 129. The method of any one of embodiments 119-128, wherein the individual has no indication of a disease, optionally wherein the disease is a cancer.

Embodiment 130. The method of any one of embodiments 119-129, wherein detecting or analyzing the immune cells that bind to one or more display moieties comprises observing the agglutination of immune cells and MHC-multimer units.

Embodiment 131. The method of any one of embodiments 119-130, wherein detecting or analyzing the immune cells that bind to one or more display moieties comprises 1) immobilizing immune cells on a solid substrate prior to or after step a), 2) contacting the immobilized immune cells with a detection agent recognizing the display moieties under a condition sufficient for the agent to bind to the display moieties; and 3) detecting the detection agent, thereby detecting the immune cells bound to the display moieties.

### EXAMPLES

The examples below are intended to be purely exemplary of the invention and should therefore not be considered to limit the invention in any way. The following examples and detailed description are offered by way of illustration and not by way of limitation.

### Example 1

### A. Methods

### HLA Purification

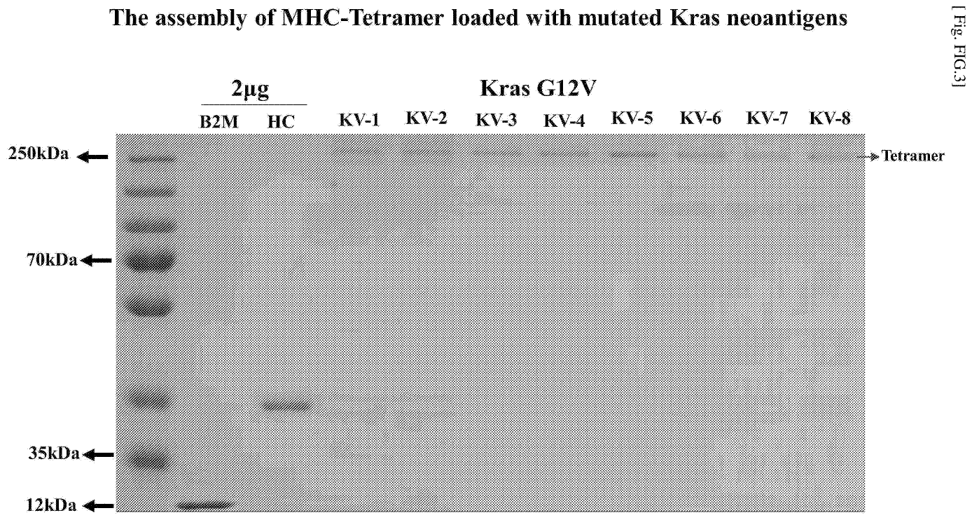

HLA-A: 02: 01, HLA-A: 24: 02, HLA-A: 11: 01, HLA-A: 03: 01, and β-2M were expressed in Escherichia coli. The purified subunits were confirmed by Coomassie blue staining. See e.g., FIG. 2.

### Kras Mutation Specific Neoantigen Library

Neoantigens comprising one of the four most common Kras mutations, including Kras G12V, Kras G12D, Kras G12R, Kras G12C were selected for building the exemplified neoantigen library. Twenty predicated neoantigens for each mutation were designed according to the features of neoantigens. These features include binding affinity with MHC molecules and/or T cells, binding stability, mutation sequence, and public neoantigens library. See e.g., FIG. 1.

### Neoantigen Synthesis

The neoantigen library was synthesized by Nanjing Peptide Biotech Ltd. The purity of peptide was above 95%.

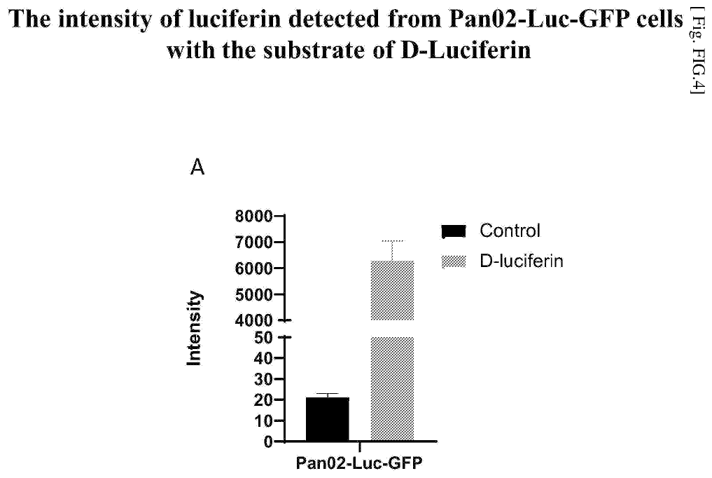

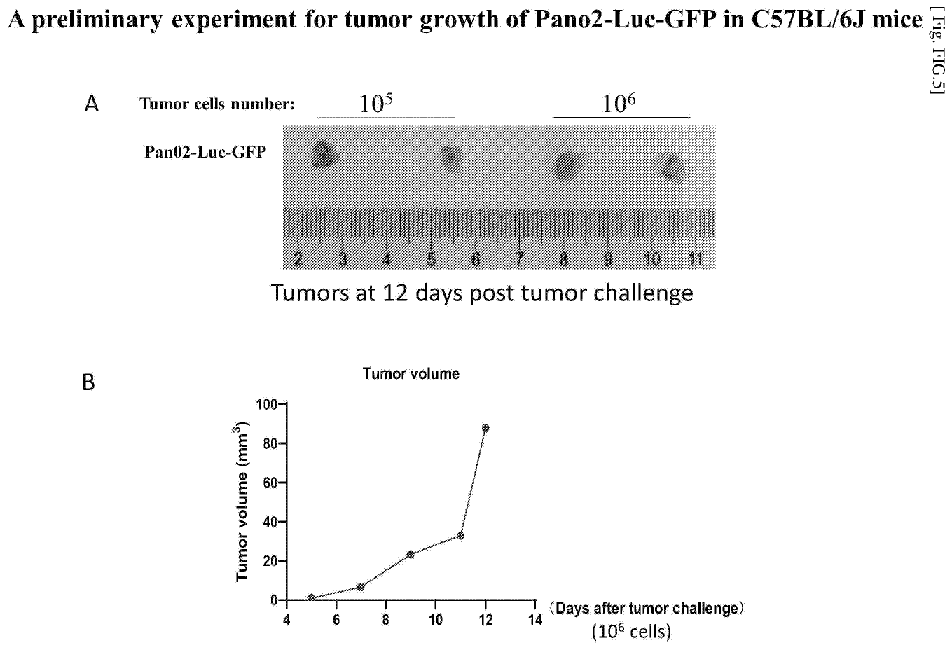

Tetramers for specific neoantigens were assembly in vitro. The brief procedure was as following.
1. Put a 100 ml beaker with sterilized magnetic pole into a 4 °C refrigerator for precooling. Add reduced glutathione to make its final concentration 5 mM; Add oxidized glutathione, and the final concentration is 0.5 mM; The storage concentration is 100 mM (prepared with isopropanol) and the final concentration is 0.2 mM in the −20 °C refrigerator.
2. Take 100 nmol MHC heavy chain and 200 nmol MHC light chain, respectively, and thaw them (previously dissolved with 6M guanidine hydrochloride). Add 5 mL injection buffer (see solution configuration), and use a pipette to mix it evenly to avoid precipitation caused by high local protein concentration.
3. Use 100 ul DMSO to fully dissolve the mixture of the peptides (e.g., mixture of neoantigens comprising G12V, G12D, G12R, or G12C) with purity ≥95%. Depending on the solubility of the peptides, 30 mg of the peptide was dissolved in a minimum volume (e.g., 1 mL) of DMSO.
4. Put the folding system (i.e., the system for assembling MHC-peptide tetramer mixture) into a magnetic stirrer at 4 °C and stir at high speed. Use a 1 mL pipette to slowly drop the peptides into the stirring folding system. Avoid excessive local peptide concentration and add it drop by drop.
5. Put the prepared MHC heavy chains and MHC light chains on the ice to be precooled for use. After the preparation is completed, use a 1 mL sterile pipette to add 5 mL light chain and heavy chain suspension to the high-speed stirred reaction system. Avoid precipitation caused by excessive local protein concentration. Use the same method when adding the heavy chain into the folding system, and add the folding buffer as close to the mixing rod as possible.
6. Return the folding reaction system to 4 °C and incubate it for 12 hours (overnight). During this period, the magnetic stirrer was used to stir gently (the rotation speed was controlled at 150-200 rpm/min) to make the folding reaction more complete.
7. After incubation (i.e., assembly of MHC-peptide mixture), add 1 μM MHC heavy chain prepared as in step 2 per method described in step 5, and sufficiently incubate for 10 hours at 4 °C.
8. After incubation (for example, before leaving at night), add an additional 1 μM heavy chain as described above (step 5) and return to 10 °C for at least overnight and at most 5 days.
9. Purify the assembled tetramers by anion-exchange chromatography. See e.g., FIG. 3 for exemplified successfully assembled MHC tetramers with neoantigen peptides comprising G12V mutation. MHC tetramers with neoantigen peptides comprising G12R, G12D or G12C were also generated. Construction of pancreatic tumor cells line expressing multiple Kras mutation specific neoantigens.

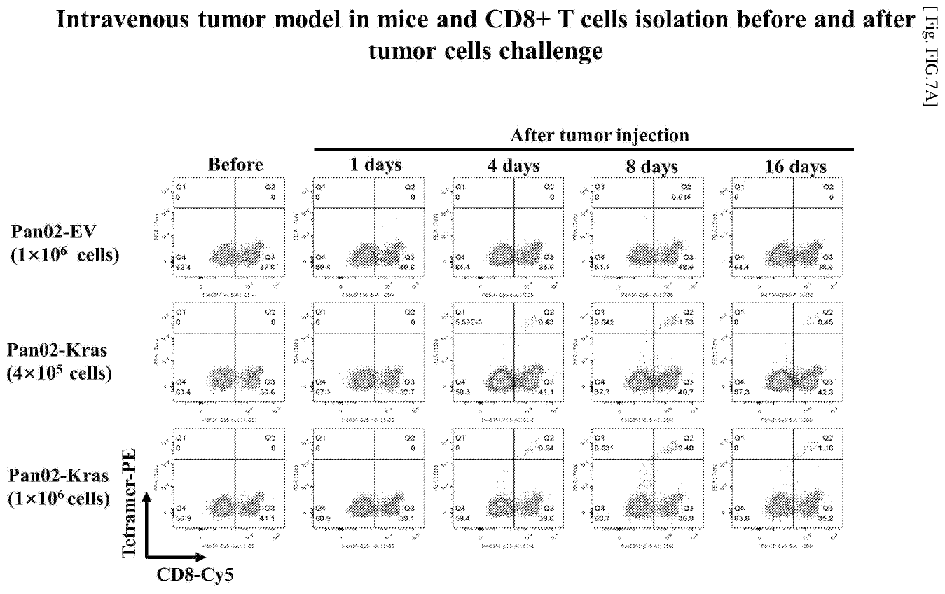

The five most common Kras mutation in pancreatic cancer were co-overexpressed in one plasmid and transfected into cells to stably expression in pancreatic tumor cells Pan02-Luc-GFP or 266-6-luc to produce excess Kras mutation specific neoantigens. The expression of transfected Kras mutant sequence were detected by PCR and mass spectrum. See e.g., FIG. 6.

### Animal Model of Tumor Cells Co-Overexpressing Five Most Common Kras Mutations

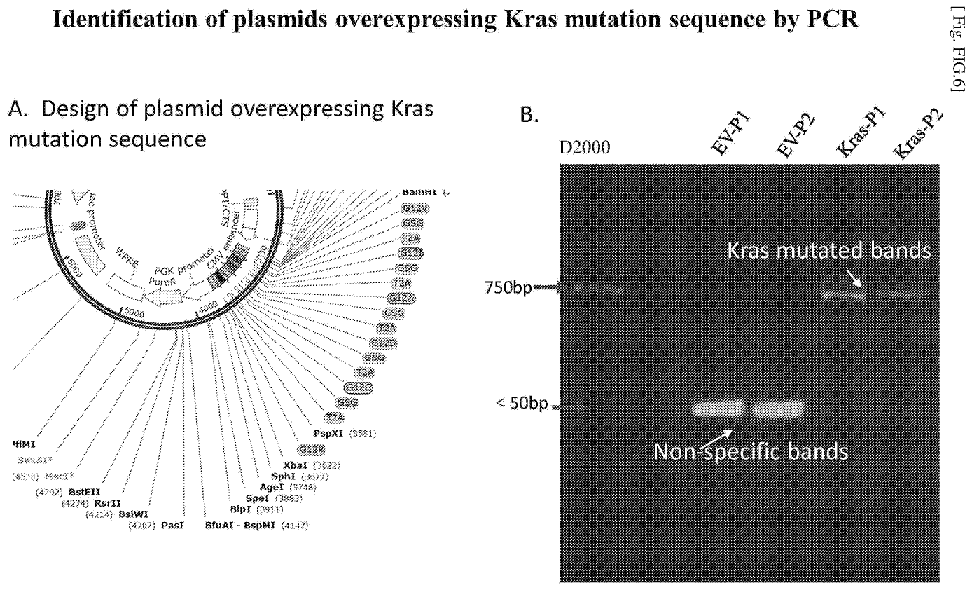

Subcutaneous tumor model: Tumor cells Pan02-Luc-GFP (e.g., 4×10⁵) which have different gradient antigen peptide combinations were inoculated subcutaneously in C57BL/6J mice. The existence of T cells targeting neoantigens comprising Kras mutations were evaluated by contacting peripheral blood cells with tetramers targeting specific antigen library prepared as above various days post inoculation respectively to determine the time point of early diagnosis on tumor size and immune stimulation time. The tumor size was recorded by bioluminescence. See FIG. 5.

Intravenous model: Tumor cells which have different gradient overexpression neoantigen peptide combinations were inoculated through the tail vein. The existence of T cells targeting neoantigens comprising Kras mutations were evaluated by incubating tetramers targeting specific neoantigen library prepared as above on day 0, day 1, day 4, day 8, day 16, and day 24 respectively, so as to determine the time point of early diagnosis on tumor size and immune stimulation time. The number of residual tumor cells in the peripheral system were monitored in real time through the autofluorescence of the inoculated cells.

The Collection of Peripheral Blood from Pancreatic Cancer Patients and T Cells Capture

The fresh blood was collected from pancreatic cancer patients and the red blood cells were lysed by ACK lysis buffer for 5 min at room temperature. Then, the white cell pellet was washed for two times before cell staining with cool PBS on ice. Fcblock antibody was used to pretreated samples for 10 min at 4 °C, and the fluorescence conjugated primary antibodies targeting CD45, CD3e, or CD8 along with Tetramer were added for cell specific staining for 30 min in dark at 4 °C. After staining, the cells were washed by cool PBS (containing 2% FBS) for two time. Finally, the cells were detected by flow cytometry immediately and the data was analyzed by Flow Jo V10 software.

### B. Results

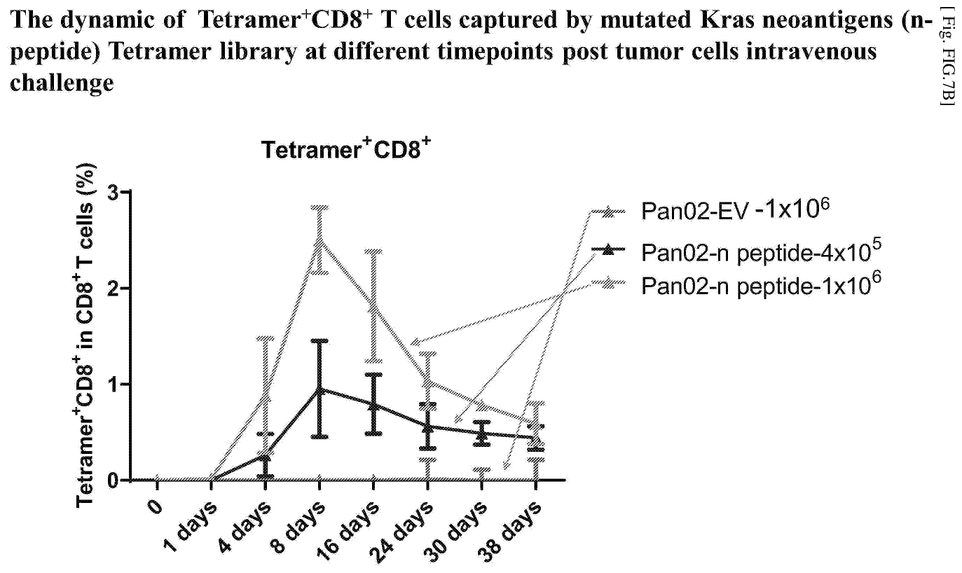

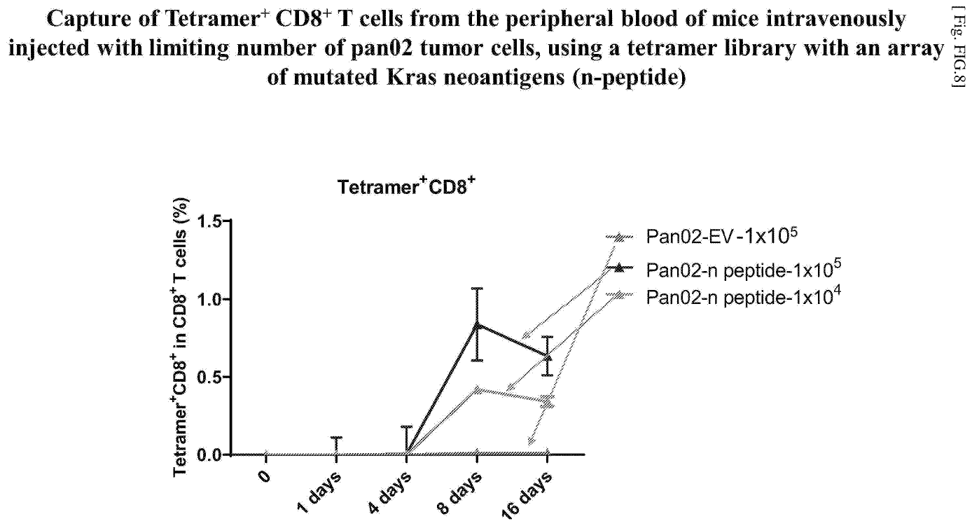

As shown in FIGS. 7A-7B, 8, 9A-9B, Kras mutation associated neoantigen-specific T cells were successfully detected in mice intravenously or subcutaneously inoculated with tumor cells (of as low as 10⁴ cells, see, e.g., FIG. 8) expressing Kras mutation associated neoantigens as early as day 4 after inoculation (see e.g., FIG. 9A-9B) which is prior to the any detection via bioluminescence (see e.g., FIG. 10A-10B), or in pancreatic cancer patients harboring Kras mutations (e.g., see FIGS. 11A-11C and 12). These results demonstrated successful applications of a tetramer based screening platform for early cancer screening.

### Example 2. Using ELISA to Detect Neoantigen Specific CD8+ T Cells for Early Cancer

### Procedure I.

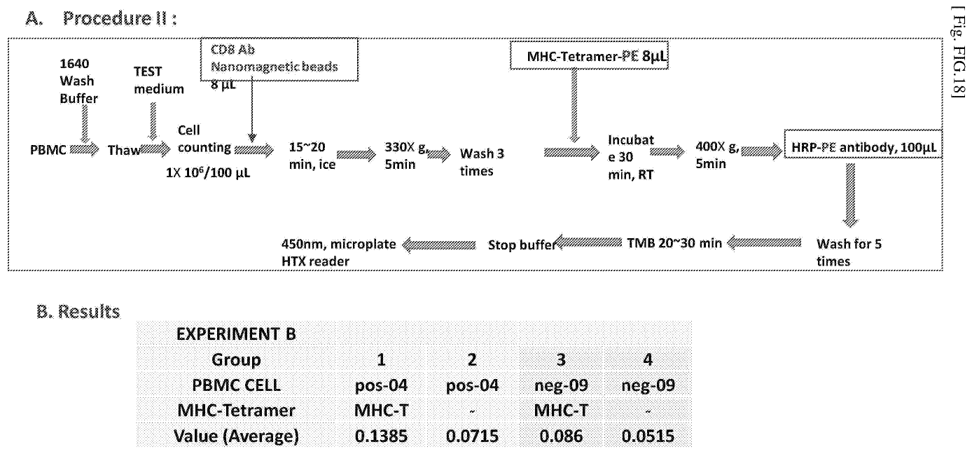

See FIG. 17 panel A for the specific method. Specifically, the cells were mixed with MHC-Tetramer and then incubated with CD8 antibody conjugated magnetic beads. HRP-β2-microglobulin antibody was the color detection antibody used to identify MHC-Tetramers binding CD8+ T cells.

Group 2 and 5 showed a positive result without adding to MHC-Tetramers and there was no significant difference. The results indicated an unspecific signal caused by HRP-β2-microglobulin antibody. Group 7 showed a positive signal from just mixing the PBMC cells along with HRP-B2-microglobulin antibody. This implies that HRP-β2-microglobulin antibody might ubiquitously bind with PBMC cells. Procedure I is not an ideal method.

### Procedure II.

See FIG. 18 panel A for the specific method. Specifically, the cells were mixed with CD8 antibody conjugated magnetic beads firstly and then incubated with MHC-Tetramers. HRP-PE antibody was the detection antibody used to identify MHC-Tetramers binding CD8+ T cells.

This preliminary data showed that this procedure might work, however, the specificity could be further optimized.

### Procedure III.

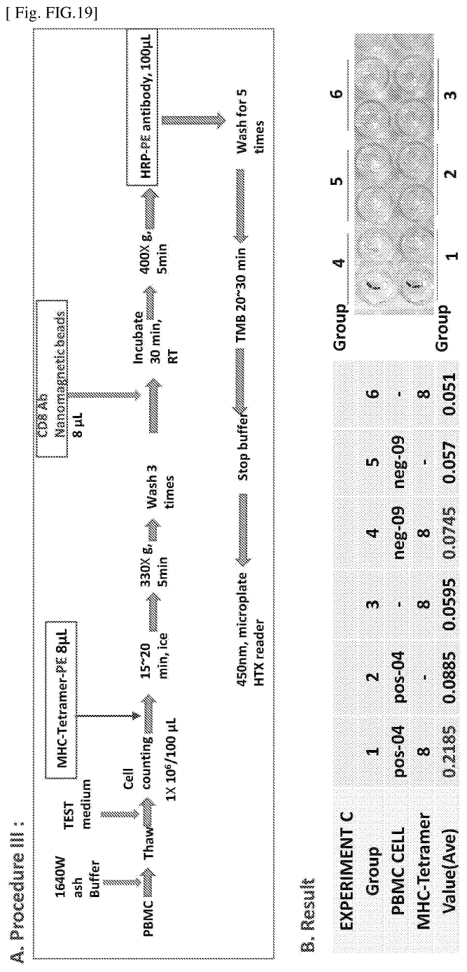

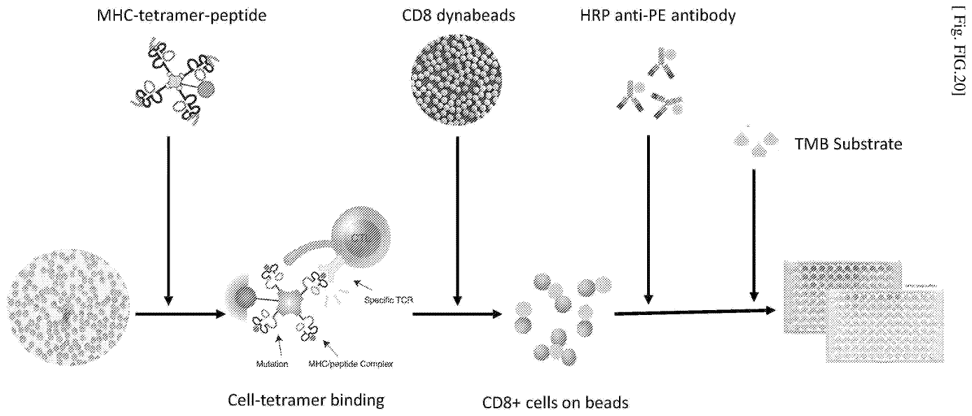

See FIG. 19 panel A for the specific method. Specifically, the cells were mixed with MHC-Tetramer and then incubated with CD8 antibody conjugated magnetic beads. HRP-PE antibody was the color detection antibody used to identify MHC-Tetramers binding CD8+T cells.

As shown in FIG. 19 panel B, there is a 3 folds difference between Group 1 and 4. The presentative photo of ELISA plates of the result was also shown. It is important to note that this procedure III has been independently verified. The results demonstrate that the method is robust and effective in detecting antigen-specific T cells in e.g., a blood sample.

### Example 3

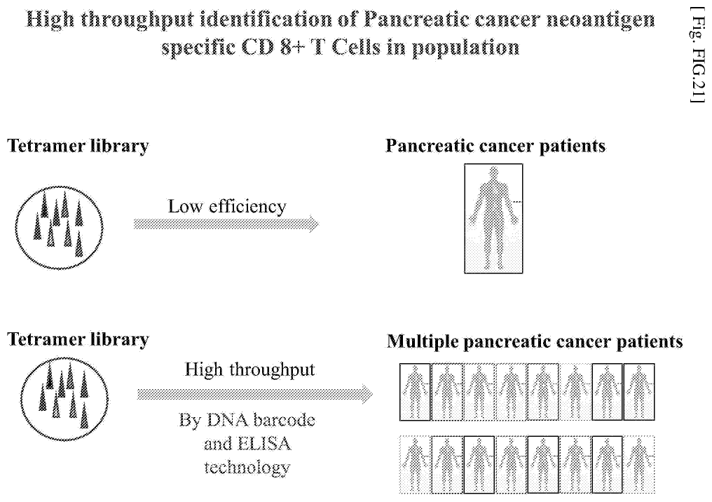

PBMC were thawed and washed and resuspend in medium. Cells (e.g., 1×10\hat{ }6 cells 100 uL) were then mixed tetramer and incubated at room temperature. Cells were washed or the bead were added (optional). Beads were then incubated with cells for about 20 min. Cells bound to beads were washed using a magnet and then anti-PE antibody was added. Antibody binding was carried out for about 1-2 hours. Cells were then washed again using a magnet and unbound antibody was washed away. TMB was added and color was developed. Stop Solution was added to stop the reaction. Absorbance at 450 nM (OD) was measured. See e.g., FIG. 20.

### 3A.

PBMC were from a patient who was infected by hCMV. A PBMC sample with no reactivity against CMV pp65 tetramer was used as negative control. PE-Tetramer-CMV pp65 (NLVPMVATV), Dynabeads CD8 and HRP conjugated anti-PE antibody were also used.

Results were shown in the Table 2 below. As shown, antigen-specific CD8+ T cells from CMV infected patient were identified by its corresponding peptide presenting tetramers. Extra wash (wash away unbound tetramers) will reduce background and may hence increase the POS/NEG ratio.

TABLE 2

| | | | POS/NEG |
|---|---|---|---|
| NEG* | POS | | ratio |
| 0.177 | 0.682 | Rep 1 | 3.9 |
| 0.151 | 0.71 | Rep 2 | 4.7 |
| 0.095 | 0.475 | Extra wash- | 5 |
| | | Rep 1 | |
| 0.07 | 0.48 | Extra wash- | 6.9 |
| | | Rep 2 | |

*NEG: PBMC not reacting with CMV pp65 tetramer

### 3B.

In this experiment, human anti-MART-1 antigen specific T cells and anti-WT-1 antigen specific T cells were used. Other materials include PE-Tetramer-MART-1 (ELAGIGILTV), PE-Tetramer-WT-1 (RMFPNAPYL), PE-Tetramer-CMV pp65 (used in 3A) as control, dynabeads CD8, HRP-conjugated anti-PE antibody.

Results as shown in the Table 3 below demonstrate that specific CD8+ T cells of cancer origin could be effectively identified by their corresponding peptide presenting tetramers using this method.

TABLE 3

| | | | POS/NEG |
|---|---|---|---|
| | Ctrl | POS | ratio |
| WT-1 | 0.097 | 3.773 | 38.9 |
| MART-1 | 0.097 | 3.389 | 34.9 |

### 3C

In this experiment, PBMC from a Colorectal Cancer patient with G12D mutation was used. Other materials include PE-Tetramer with a pool of 10 KRAS G12D mutation derived peptides, PE-Tetramer-CMV pp65 (used in 3A and 3B) as control, dynabeads CD8, HRP-conjugated anti-PE antibody.

Results as shown in the Table 4 below demonstrate neoantigen-specific CD8+ T cells from cancer patients can be effectively identified by this methods.

TABLE 4

| | | POS/NEG |
|---|---|---|
| Ctrl* | POS* | ratio |
| 0.095 | 0.168 | 1.8 |

### 3D.

In this Experiment, Antigen-Specific T Cells (ASTC) were Acquired from Patients'

PBMCs. Prior to the tests, these ASTC cells were activated (or reactivated) by antigen-presenting cells (APCs) with corresponding peptides (e.g., MART-1 (ELAGIGILTV) or WT-1 (RMFPNAPYL)). The resulting activated ASTC cells (e.g., ASTC-MART-1 or ASTC-WT-1) were used in the subsequent ELISA tests at 0.21×10⁶ ASTC-MART-1 cells/reaction and 0.25×10⁶ ASTC-WT-1 cells/reaction, respectively. Other materials include PE-Tetramer-MART-1 (ELAGIGILTV), PE-Tetramer-WT-1 (RMFPNAPYL), PE-Tetramer-CMV pp65 (used in 3A) as control, dynabeads CD8, HRP-conjugated anti-PE antibody.

Results as shown in the Table 5 below demonstrate that specific CD8+ T cells of cancer origin could be effectively identified by their corresponding peptide presenting tetramers using this method.

TABLE 5

| | | | POS/NEG |
|---|---|---|---|
| | Ctrl | POS | ratio |
| WT-1 | 0.053 | 1.508 | 28.5 |
| MART-1 | 0.042 | 1.401 | 33.4 |

### 3E.

In this experiment, G12D cells were used. Ten individual PE-Tetramer complexes were made by MBL's PE-Tetramer peptide exchange with MBL QuickSwitch Quant HLA-A*01: 01 Tetramer Kit-PE using the ten G12D neoantigen peptides in Table 6. The resulting ten individual G12D PE-Tetramer complexes were then mixed at equal weight ratio to form the G12D-PE-Tetramer mix. Other materials include PE-Tetramer-CMV pp65 (used in 3A) as control, dynabeads CD8, HRP-conjugated anti-PE antibody. The control cells are PBMC from a patient who was infected by hCMV and showed reactivity against CMV pp65 peptide.

Results demonstrate that specific CD8+ T cells (i.e. G12D cells) could be effectively identified by their corresponding peptide presenting tetramers using this method.

TABLE 6
List of ten G12D neoantigen peptides.

KLVVVGADGV
LVVVGADGV
VVGADGVGKSA
VGADGVGKSAL
VGADGVGKSA
VVVGADGV
VVVGADGVGK
VVGADGVGK
KLVVVGADG
VVGADGVGKS

### 3F.

In this experiment, G12V cells from clinical samples were used. Thirteen individual PE-Tetramer complexes were made by MBL's PE-Tetramer peptide exchange with MBL QuickSwitch Quant HLA-A*01: 01 Tetramer Kit-PE using the thirteen G12V neoantigen peptides in Table 8. The resulting thirteen individual G12V PE-Tetramer complexes were then divided into 4 groups (e.g., peptide group 2/3/4; peptide group 6/8/9; peptide group Oct. 11, 2012; peptide group 13/14/15/16), and mixed at equal weight ratio within each group to form four groups of G12V-PE-Tetramer mixes. Other materials include PE-Tetramer-CMV pp65 (used in 3A) as control, dynabeads CD8, HRP-conjugated anti-PE antibody. The control cells are PBMC from a patient who was infected by hCMV and showed reactivity against CMV pp65 peptide.

Results as shown in the Table 9 below demonstrate that specific CD8+ T cells (i.e. G12V cells) could be effectively identified by their corresponding peptide presenting tetramers using this method. Different peptide groups have shown different binding affinity to CD8+ T cells in this sample. Additionally, this experiment also showed that peptides have community effect (e.g., peptide groups pulled down more T cells than individual peptides).

TABLE 8
List of ten G12V neoantigen peptides.

| | |
|---|---|
| KV-2 | KLVVVGAVG |
| KV-3 | KLVVVGAVGV |
| KV-4 | KLVVVGAVGVG |
| KV-6 | LVVVGAVGV |
| KV-8 | LVVVGAVGVGK |
| KV-9 | VVVGAVGV |
| KV-13 | VVGAVGVG |
| KV-14 | VVGAVGVGK |
| KV-15 | VVGAVGVGKS |
| KV-16 | VVGAVGVGKSA |

TABLE 9

| | POS/NEG |
|---|---|
| Peptide groups (KV) | ratio |
| 2/3/4 | 3.10 |
| 6/8/9 | 4.58 |
| 13/14/15/16 | 2.23 |

The resulting thirteen individual G12V PE-Tetramer complexes were also individually tested against G12V cells, as shown in Table 10 below.

| | POS/NEG |
|---|---|
| Peptides (KV) | ratio |
| 2 | 1.80 |
| 3 | 1.22 |
| 4 | 1.98 |
| 6 | 3.28 |
| 8 | 3.92 |
| 9 | 1.28 |
| 13 | 1.90 |
| 14 | 1.30 |
| 15 | 0.94 |
| 16 | 1.15 |

Results as shown in the Table 10 below demonstrate that specific CD8+ T cells (i.e. G12V cells) could be effectively identified by their corresponding peptide presenting tetramers using this method. Different peptides have also shown different binding affinity to CD8+ T cells in this sample.

### Example 4

The examples exemplify high throughput screening for antigen specific immune cells (e.g., CD8+ T cells) by ELISA using DNA barcode technology.

### 4A. Tetramer Library Construction for Early Pancreatic Cancer Detection.

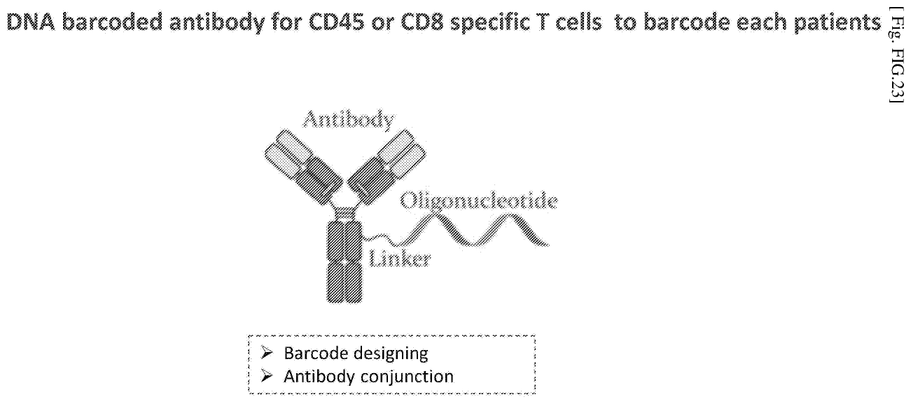

As shown in FIG. 22, a tetramer library was constructed for detecting early pancreatic cancer. The antigenic peptides include those with KRAS mutations (such G12R, G12C, G12D), TP53 mutations (such as R248W and C141Y). Various MHC subtypes were included (e.g., HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03). Exemplary neoantigen library can include more than 1000 neoantigens. Exemplary tetramer library can have more than 10000 unique monomers.

### 4B. MHC-Peptides Monomer Assembly and Production of Exemplary MHC Multimer Units (Display Moiety)

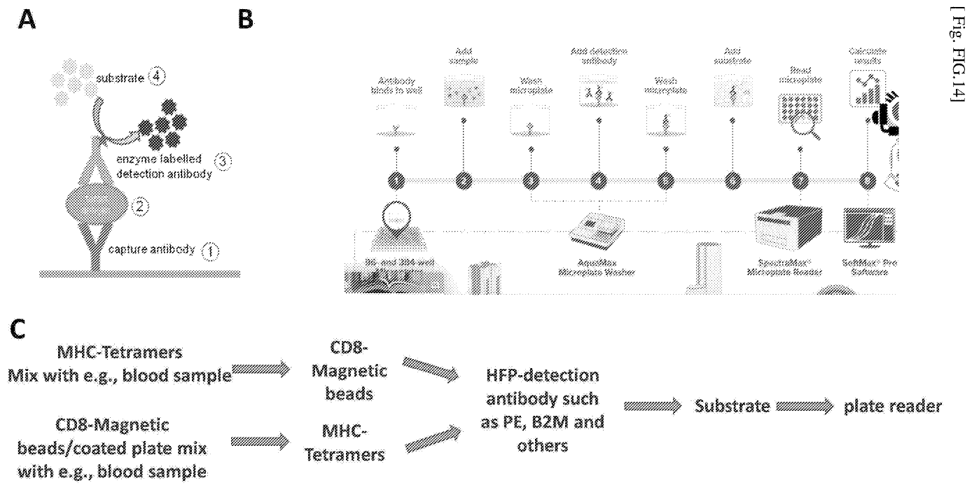

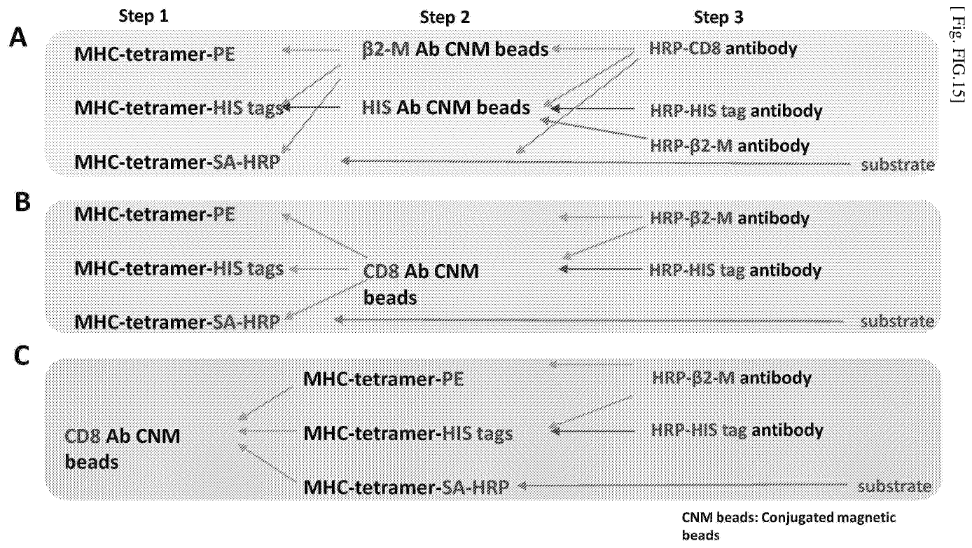

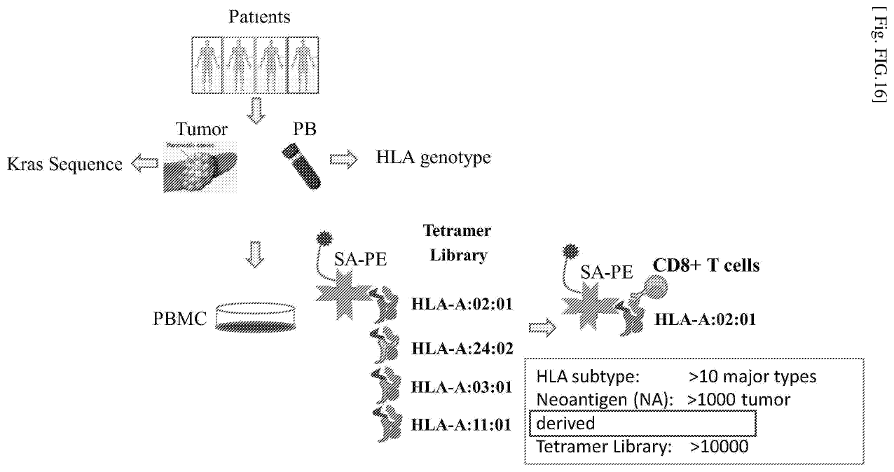

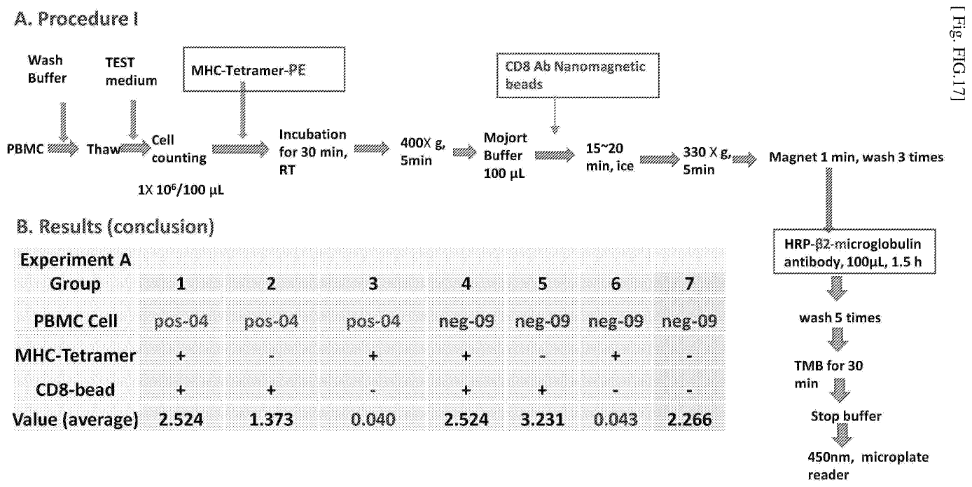

See e.g., FIG. 13. The heavy chain and light chain of MHC were purified. The subunits of MHC were refolded with pre-screened tumor specific neoantigens library in vitro to form MHC-peptides monomers. Monomers were biotinylated and excess biotin in the reaction system were removed. Non-denature gels in non-reducing electrophoresis system were run to confirm the formation of monomer or Tetramer.

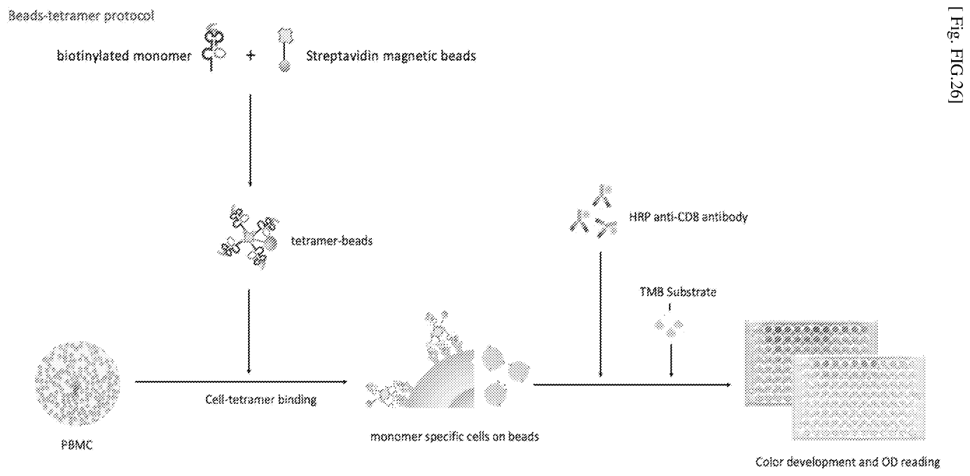

Streptavidin and magnetic beads were thoroughly mixed and then washed. Biotinylated monomers were then added for overnight incubation. See e.g., FIG. 25.

4C. Barcoding of CD45 Antibody with DNA Oligos to Perform High-Throughput Screening of Patients

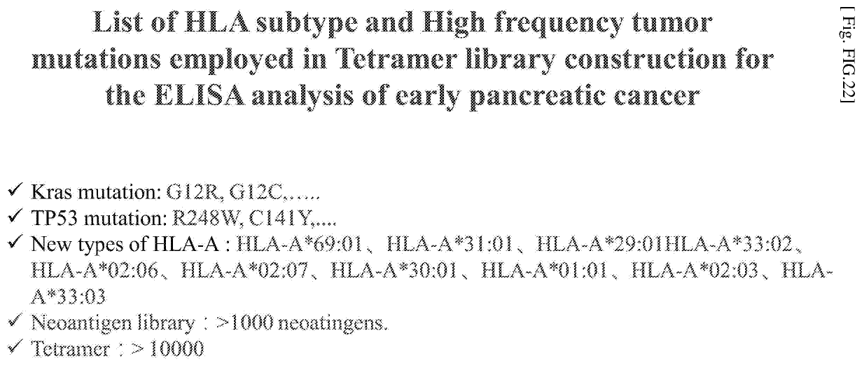

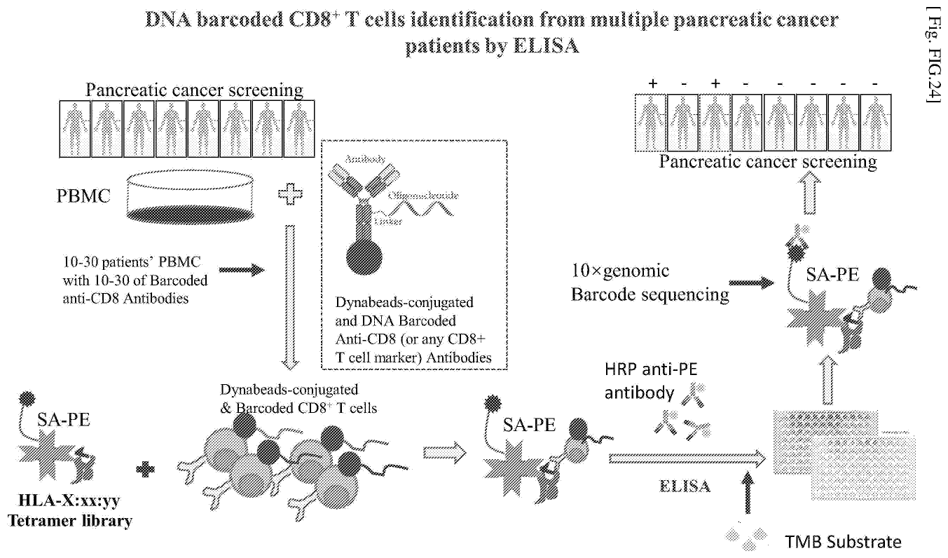

Unique DNA barcodes were designed and a plurality of CD45 antibody with unique DNA barcodes were produced. See e.g., FIGS. 21 and 23. The threshold of cell numbers for individuals were tested to distinguish the samples through DNA barcodes.

### 4D. Early Diagnosis of Pancreatic Cancer Based on Beads-MHC Multimer and ELISA in Human

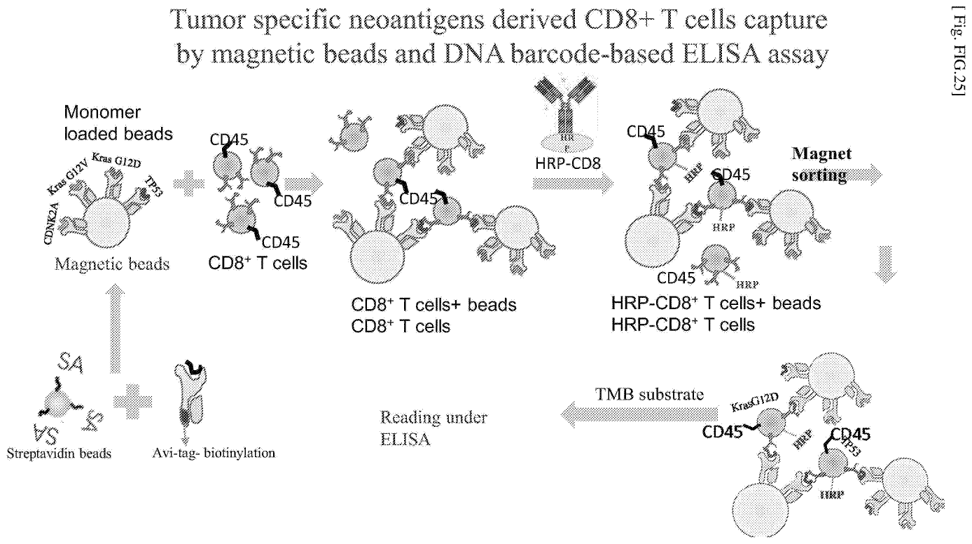

As illustrated in FIG. 24, peripheral blood of 20 potential pancreatic cancer patients were collected, and the PBMC was isolated. PBMCs from each individual were separately incubated with 20 unique DNA barcoded CD45 antibodies (e.g., for about 30 min). Cells were washed to remove excess CD45 antibodies. The barcode labelled PBMCs were pulled together with 4×10⁵ cells per patients, and resuspends in medium. CD8-HRP antibody was added to cells for about 30 mins. Previously made Beads-SA-MHC-peptide monomers were added into PBMCs and incubated for about 1-2 hours. After that, the magnet was used to separated beads binding CD8⁺ T cells (e.g., Beads-SA-MHC-peptide monomers-CD8-HRP cells) from beads-free cells, which were washed out.

An alternative method as illustrated in FIG. 25 can also be used.

ELISA methods described in e.g., Examples 2 and 3 were used to identify pancreatic cancer patients.

Alternatively or additionally, sequencing (e.g., 10× genomics) can be used to identify patients that have neoantigen specific CD8⁺ T cells.

### Example 5

### 5A.

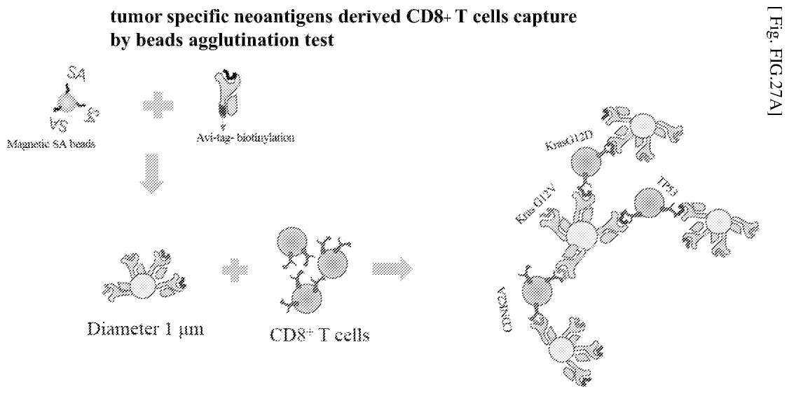

Tetramer-beads (i.e., pMHC beads-tetramers) were prepared according to e.g., the protocol in Example 4B. PBMC were thawed and washed and resuspend in medium. Cells (e.g., 1×10\hat{ }6 cells 100 uL) were then mixed tetramer and incubated at room temperature. Tetramer-beads were then incubated with cells for about 50 min. Cells bound to beads were washed using a magnet and then HRP conjugated anti-CD8 antibody was added. Antibody binding was carried out for about 1-2 hours. Cells were then washed again using a magnet and unbound antibody was washed away. TMB was added and color was developed. Stop Solution was added to stop the reaction. Absorbance at 450 nM (OD) was measured. See e.g., FIG. 26.

In this experiment, G12V cells from clinical samples were used. Individual PE-Tetramer complexes were made by Biolegend's Flex-T™ HLA-A*02: 01 Monomer UVX using the thirteen G12V neoantigen peptides in Table 8. The resulting individual G12V PE-Tetramer complexes were then used to make the corresponding tetramer-beads. Other materials include tetramer-beads conjugated with PE-Tetramer-CMV pp65 (used in 3A) as control, HRP-conjugated anti-CD8 antibody. The control cells are PBMC from a patient who was infected by hCMV.

Results as shown in the Table 11 below demonstrate that specific CD8+ T cells (i.e. G12V cells) could be effectively identified by their corresponding peptide presenting tetramer-beads using this method. Different peptides have shown different binding affinity to CD8+ T cells in this sample.

TABLE 11

| Peptides (KV) | POS/NEG |
|---|---|
| 2 | 3.05 |
| 3 | 2.62 |
| 4 | 1.25 |
| 11 | 2.78 |
| 14 | 2.29 |

### 5B.

The pHLA monomers (i.e., human peptide-HLA complex) were separately prepared with eighteen KRAS G12V neoantigens, as shown in Table 12 below, modified according to e.g., protocol in Example 1A. Briefly, in a 20 mL refolding system containing Arginine, Glutathione oxidized, Glutathione reduced and PMSF, add 0.6 mg peptides and 0.468 mg B2M. HLA was added in three batches at 0.694, 0.347 mg, and 0.347 mg. Then, a series of purification steps (Ultrafiltration to concentrate refolded complex; Dialysis of refolded complex; Two-step ultrafiltration to concentrate pHLA complex) were performed to isolate the pMHC monomers. The pHLA monomers were then biotinylated and dialyzed to remove excess biotin. Tetramer-beads (i.e., pHLA beads-tetramers) were prepared with a mixture of the eighteen pHLA monomers at equal weight ratios, according to the protocol in Example 4B.

G12V cells from clinical samples were used and were pre-treated with 100 nM drug Dasatinib (N-(2-chloro-6-methylphenyl)-2-[[6-[4-(2-hydroxyethyl)-1-piperazinyl]-2-methyl-4-pyrimidinyl]amino]-5-thiazolecarboxamide, monohydrate) before tests. Cells (e.g., 1×10\hat{ }6 cells 100 uL) were then mixed tetramer-beads and incubated at room temperature for about 50 min. Cells bound to tetramer-beads were washed using a magnet and then HRP conjugated anti-CD8 antibody was added. Antibody binding was carried out for about 1-2 hours. Cells were then washed again using a magnet and unbound antibody was washed away. TMB was added and color was developed. Stop Solution was added to stop the reaction. Absorbance at 450 nM (OD) was measured. See e.g., FIG. 26. Other materials include tetramer-beads conjugated with PE-Tetramer-CMV pp65 (used in 3A) as control, HRP-conjugated anti-CD8 antibody. The control cells are PBMC from a patient who was infected by hCMV.

TABLE 12
KRAS G12V neoantigens.

| | |
|---|---|
| KV-1 | KLVVVGAV |
| KV-2 | KLVVVGAVG |
| KV-3 | KLVVVGAVGV |
| KV-4 | KLVVVGAVGVG |
| KV-5 | LVVVGAVG |
| KV-6 | LVVVGAVGV |
| KV-8 | LVVVGAVGVGK |
| KV-9 | VVVGAVGV |
| KV-10 | VVVGAVGVG |
| KV-11 | VVVGAVGVGK |
| KV-12 | VVVGAVGVGKS |
| KV-13 | VVGAVGVG |
| KV-14 | VVGAVGVGK |
| KV-15 | VVGAVGVGKS |
| KV-16 | VVGAVGVGKSA |
| KV-17 | VGAVGVGK |
| KV-18 | VGAVGVGKS |
| KV-19 | VGAVGVGKSA |

Results as shown in the Table 13 below demonstrate that specific CD8+ T cells (i.e. G12V cells) could be effectively identified by their corresponding peptide presenting tetramer-beads using human peptide-HLA complex in this method.

TABLE 13

| | | POS/NEG |
|---|---|---|
| | OD | Ratio |
| G12V cells | 0.292 | 2.06 |
| Control cell | 0.142 | |

### Example 6A. Detection of Antigen Specific T Cells from Peripheral Blood Mononuclear Cells (PBMC) and Spleen Cells with Beads Loaded with Kras G12V (KV1-KV16) Neoantigen Library

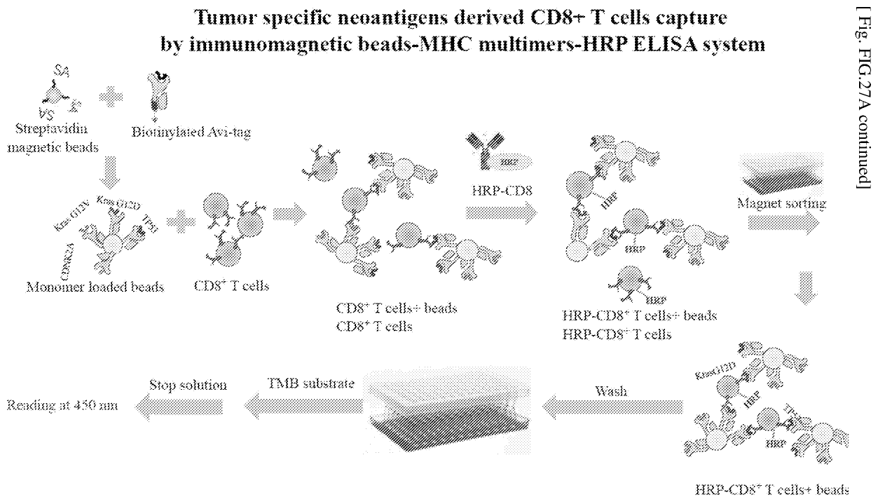

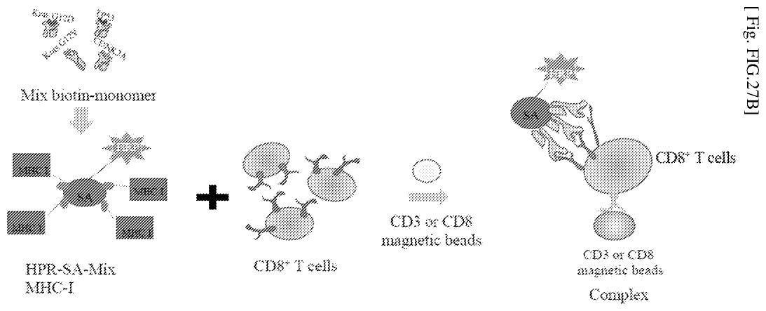

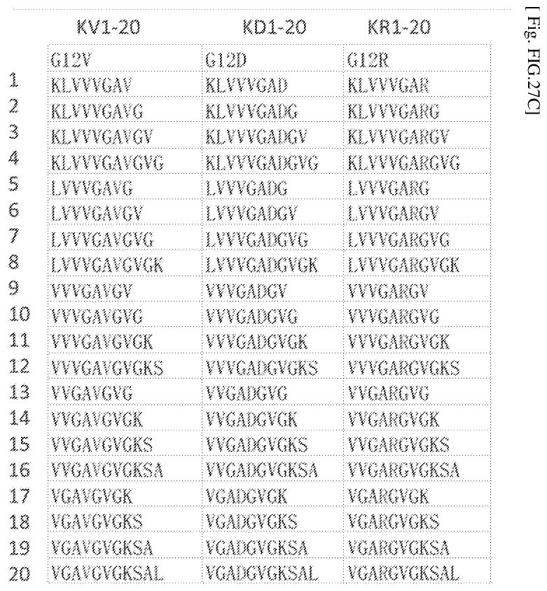

Materials: The Kras G12V (KV1-KV16; See FIG. 27B) neoantigen library was synthesized by Nanjing Peptide Biotech Ltd. The purity of peptide was above 95%. Ovalbumin-derived peptide SIINFEKL, a control peptide known to bound to H-2Kb of MHC class I. MHC heavy chain (H-2Kb) containing an Avi-tag and MHC light chain (β2m) were expressed and purified in Escherichia coli.

Preparation of MHC-peptide monomer complexes with Kras G12V (KV1-KV16) neoantigen library or Ovalbumin (OVA) derived peptide SIINFEKL as a control: Step 1. Put a 100 ml beaker with sterilized magnetic pole into a 4 °C refrigerator for precooling. Add reduced glutathione to make its final concentration 5 mM; Add oxidized glutathione, and the final concentration is 0.5 mM; The storage concentration is 100 mM (prepared with isopropanol) and the final concentration is 0.2 mM in the −20 °C refrigerator. Step 2. Take 100 nmol MHC heavy chain and 200 nmol MHC light chain, respectively, and thaw them (previously dissolved with 6M guanidine hydrochloride). Add 5 mL injection buffer (see solution configuration) and use a pipette to mix it evenly to avoid precipitation caused by high local protein concentration. Step 3. Use 100 μL DMSO to fully dissolve the mixture of the peptides (e.g., mixture of peptides comprising G12V (KV1-KV16) or Ovalbumin-derived peptide SIINFEKL). Depending on the solubility of the peptides, 30 mg of the peptide was dissolved in a minimum volume (e.g., 1 mL) of DMSO. Step 4. Put the folding system (i.e., the system for assembling MHC-peptide monomer mixture) into a magnetic stirrer at 4° Cand stir at high speed. Use a 1 mL pipette to slowly dropped the peptides into the stirring folding system. Avoid excessive local peptide concentration and added it drop by drop. Step 5. Put the prepared MHC heavy chains and MHC light chains on the ice to be precooled for use. After the preparation was completed, a 1 mL sterile pipette was used to add 5 mL light chain and heavy chain suspension to the high-speed stirred reaction system. Avoid precipitation caused by excessive local protein concentration. Use the same method when adding the heavy chain into the folding system, and added the folding buffer as close to the mixing rod as possible. Step 6. Return the folding reaction system to 4 °C and incubated it for 12 hours (overnight). During this period, the magnetic stirrer was used to stir gently (the rotation speed was controlled at 150-200 rpm/min) to make the folding reaction more complete. Step 7. After incubation (i.e., assembly of MHC-peptide monomer complexes), added 1 μM MHC heavy chain prepared as in step 2 per method described in step 5, and sufficiently incubated for 10 hours at 4 °C. Step 8. After incubation (for example, before leaving at night), add an additional 1 μM heavy chain as described above (step 5) and return to 10 °C for at least overnight and at most 5 days. Step 9. After purification of the assembled MHC-peptide monomer complexes by anion-exchange chromatography, MHC-peptide monomer complexes with Kras G12V (KV1-KV16) neoantigen library or Ovalbumin (OVA) derived peptide SIINFEKL as a control were prepared. These MHC-peptide monomer complexes comprise H-2Kb heavy chain containing an Avi-tag, β2m light chain, and Kras G12V (KV1-KV16) neoantigen library or Ovalbumin (OVA) derived peptide SIINFEKL as a control.

Biotinylation of MHC-peptide monomer complexes: The MHC-peptide monomer complexes comprise H-2Kb heavy chain containing an Avi-tag, β2m light chain, and Kras G12V (KV1-KV16) neoantigen library or Ovalbumin (OVA) derived peptide SIINFEKL as a control.

Preparation of beads loaded with Kras G12V (KV1-KV16) neoantigen library or Ovalbumin (OVA) derived peptide SIINFEKL as a control: Streptavidin were thoroughly mixed and then washed. Unconjugated magnetic beads were thoroughly washed and resuspended in PBS buffer. Next, streptavidin was added to the magnetic bead suspension at an optimal ratio to ensure sufficient coverage of the bead surface. After incubated the reaction mixture gently at room temperature, the unbound streptavidin was washed away. Then, streptavidin-coated magnetic beads were resuspended by vigorous vortexing, ensuring an even distribution. These streptavidin-coated magnetic beads were then washed and mixed with biotinylated MHC-peptide monomer. The mixture was incubated overnight at 4 °C. The resulting MHC-peptide loaded magnetic beads (exemplary display moieties) were washed thoroughly and were ready to use in the following tests.

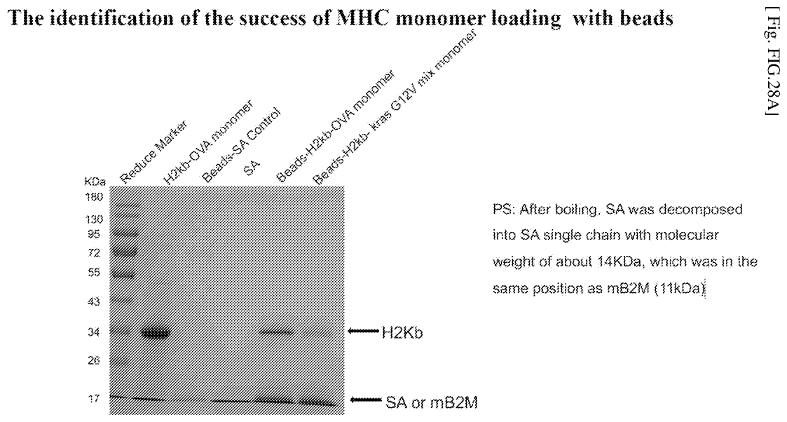

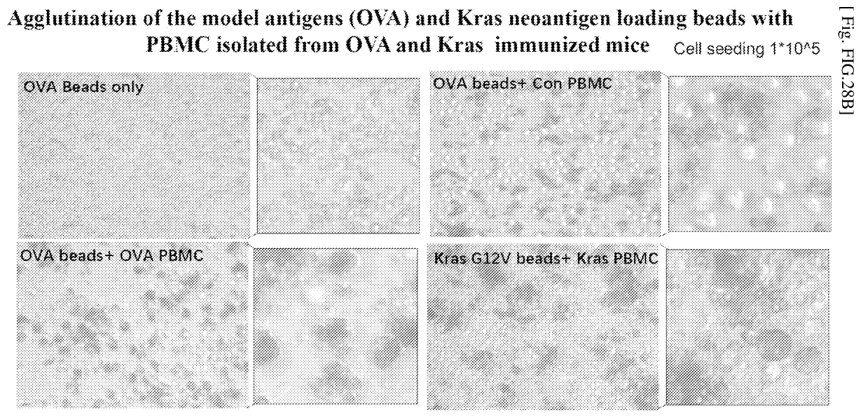

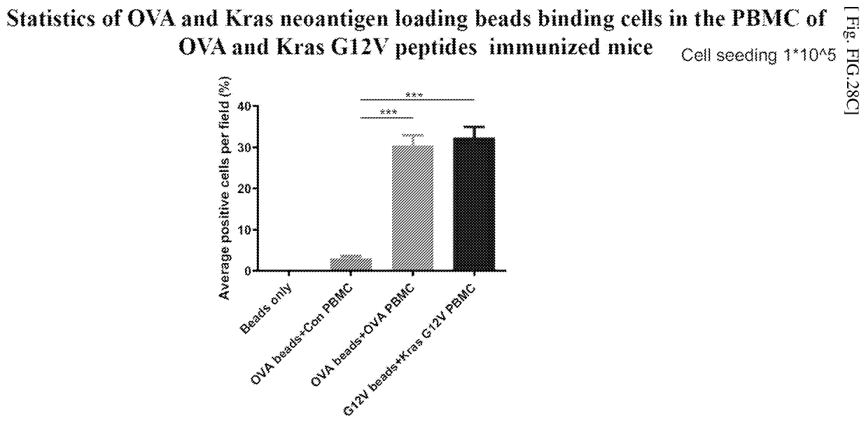

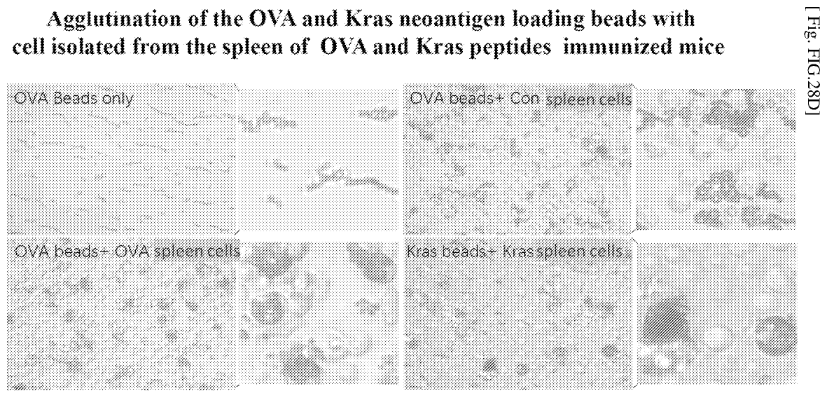

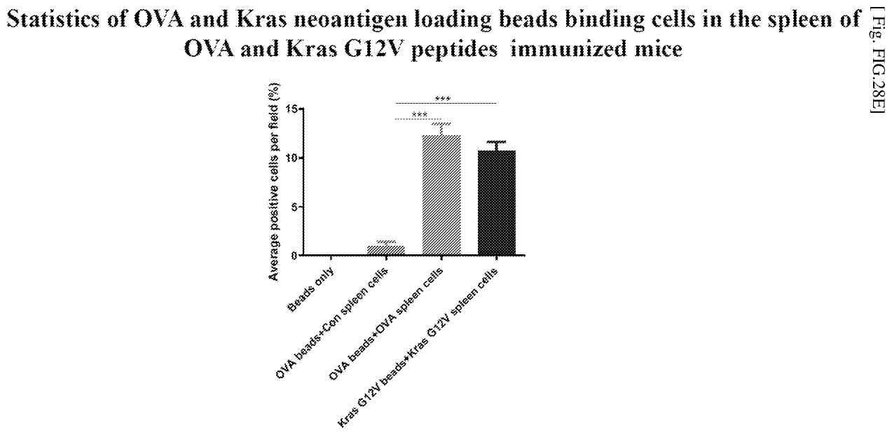

As shown in FIG. 28A, an SDS-PAGE was performed to test if the MHC-peptide monomer complexes were successfully assembled and loaded onto the magnetic beads. Lane #2 was a control for the H2Kb-OVA complexes with two expected bands: H-2Kb heavy chain containing an Avi-tag at approximately 34 kDa and β2m light chain at approximately 11 kDa. The non-covalently bound Ovalbumin (OVA) derived peptide SIINFEKL was too small to be visualized by SDS-PAGE. Lane #3 was a second control for the magnetic beads coated with streptavidin. Due to the harsh heating condition in the preparation of the SDS-PAGE samples, streptavidin decomposed into streptavidin monomer and migrated at approximately 14 kDa, at a similar location as the β2m light chain at approximately 11 kDa. Lane #4 was a third control for the streptavidin showing an expected band at approximately 14 kDa. Lane #5 was a test for the streptavidin coated magnetic beads loaded with H-2Kb-OVA complexes. Three bands were observed: H-2Kb heavy chain containing an Avi-tag, streptavidin monomer and β2m light chain. Due to the similar sizes of streptavidin monomer and β2m light chain, both proteins migrated at close location, overlapping each other, therefore forming a wide band at the bottom. The non-covalently bound Ovalbumin (OVA) derived peptide SIINFEKL was too small to be visualized by SDS-PAGE. Lane #6 was another test for the streptavidin coated magnetic beads loaded with H-2Kb Kras G12V (KV1-KV16) neoantigen library. H-2Kb heavy chain containing an Avi-tag, streptavidin monomer and β2m light chain were observed. Similar to the Ovalbumin (OVA) derived peptide SIINFEKL, non-covalently bound Kras G12V (KV1-KV16) neoantigen library was also too small to be visualized by SDS-PAGE.

In summary, FIG. 28A demonstrated that the heterogenous MHC-peptide monomer complexes, for both Kras G12V (KV1-KV16) neoantigen library and the Ovalbumin (OVA) derived peptide SIINFEKL, were successfully assembled and loaded onto the magnetic beads.

Immunization of mice with Kras G12V (KV1-KV16) neoantigen library or Ovalbumin (OVA) derived peptide SIINFEKL: Firstly, peptides of Kras G12V (KV1-KV16) neoantigen library or Ovalbumin (OVA) derived peptide SIINFEKL as a control were dissolved in an appropriate solvent (e.g., sterile PBS) to a desired concentration (e.g. 10 mg/mL). Then, a 200 μL complete immunization mixture was prepared and thoroughly mixed, including complete Freund's adjuvant, about 10 uL freshly prepared peptide solution (KV1-KV16 neoantigen library or Ovalbumin derived peptide SIINFEKL control), HBC stock, and PBS solution. Finally, the complete immunization mixture was intraperitoneally injected into the C57BL/6J mice twice, with an extended period of 10 days between injections.

The collection of Peripheral Blood Mononuclear Cell (PBMC) from immunized and control C57BL/6J mice: Using heparinized syringes or collection tubes, blood was collected from both immunized and naive C57BL/6J mice via tail vein puncture, immediately mixed with an anticoagulant to prevent clotting, and then diluted in sterile PBS. Then, this mixture was carefully layered over Ficoll-Paque in a centrifuge tube and centrifuged to separate the Peripheral Blood Mononuclear Cells (PBMCs) based on density. The PBMC layer was carefully extracted, washed in PBS, and centrifuged again to collect the PBMC cells for downstream applications.

The collection of spleen cells from immunized and control C57BL/6J mice: Following the spleen was freshly excised from a euphemized mouse, this spleen was gently pressed through a fine mesh or cell strainer using a syringe plunger or the back of a sterile pipette tip. Then an erythrocyte lysis buffer (usually containing ammonium chloride) was applied to the cell suspension to remove the erythrocytes. Finally, the remaining cells were washed and resuspended in a suitable buffer, and a purified population of spleen cells was ready for downstream applications.

Detection of Peripheral Blood Mononuclear Cells (PBMC) with Beads Loaded with Kras G12V (KV1-KV16) Neoantigen Library: In each well on 96 well plates, 1×10⁵ PBMC cells were seed in 0.1 mL assay buffer, followed by mixing with beads. The 96 well plates were then allowed to incubate at room temperature for about 0.5-1 hour. Then, the plates were imaged and the number of aggregates within each well were counted. Four conditions were performed in this assay. Condition 1 was a control for the streptavidin coated magnetic beads loaded with H-2Kb-OVA complexes. The magnetic beads were observed as round discrete particles with no aggregates present as shown in the upper left image in FIG. 28B. Condition 2 was a second control for the streptavidin coated magnetic beads loaded with H-2Kb-OVA complexes, in the presence of PBMC from naive C57BL/6J mice. Most PBMC were observed as round isolated cells with a few aggregates present as shown in the upper right image in FIG. 28B. Condition 3 was a model test for the streptavidin coated magnetic beads loaded with H-2Kb-OVA complexes, in the presence of PBMC from C57BL/6J mice immunized with Ovalbumin derived peptide SIINFEKL. Nearly all cells in the microscopic field formed large aggregates, indicating that the H-2Kb-OVA loaded magnetic beads were extensively crosslinked with the immunized PMBC cells. This result, as shown in the lower left image in FIG. 28B, demonstrated the PBMC cells (e.g., T cells) have produced receptors on the cell surface that were able to recognize the Ovalbumin derived peptide SIINFEKL. Condition 4 was a test for the streptavidin coated magnetic beads loaded with H-2Kb-OVA with Kras G12V (KV1-KV16) neoantigen library complexes, in the presence of PBMC from C57BL/6J mice immunized with the same Kras G12V (KV1-KV16) neoantigen library. Nearly all cells in the microscopic field formed large aggregates, indicating that the H-2Kb-(Kras G12V KV1-KV16) loaded magnetic beads were also extensively crosslinked with the immunized PMBC cells. This result, as shown in the lower right image in FIG. 28B, demonstrated the PBMC cells (e.g., T cells) have produced receptors on the cell surface that were able to recognize the Kras G12V KV1-KV16. Quantification of the aggregates averaging six fields per tested condition were shown in FIG. 28C.

Detection of Spleen Cells with Beads Loaded with Kras G12V (KV1-KV16) Neoantigen Library: In each well on 96 well plates, 1×10⁵ PBMC cells were seed in 0.1 mL assay buffer, followed by mixing with magnetic beads. The 96 well plates were then allowed to incubate at room temperature for 0.5-1 hour. Then, the plates were imaged and the number of aggregates within each well were counted. Similar to the test performed in the test with PBMC cells, four conditions were also performed in this assay. In both controls as shown in FIG. 28D for Condition 1 and 2, no or few aggregates were observed as shown in the upper left and right images in FIG. 28D. In the tests describe in the lower left and right images in FIG. 28D for Condition 3 and 4, extensive aggregates were observed when spleen cells from C57BL/6J mice, immunized with peptides (either the Ovalbumin-derived peptide SIINFEKL or the Kras G12V (KV1-KV16) neoantigen library), were mixed with magnetic beads loaded with matching peptide complex. This result provided additional evidence demonstrating the cells (e.g. CD8+ cells) have produced receptors on the cell surface that were able to recognize the Kras G12V KV1-KV16. Quantification of the aggregates averaging six fields per tested condition were shown in FIG. 28E.

In summary, the results above with either the PMBC or the spleen cells from immunized mice demonstrated that the exemplified methods discussed above were effective in detecting antigen-specific immune cells by allowing the antigen-specific cells to aggregate upon contact with the MHC-peptide complex bait composition.

### Example 6B. Tests of Spleen Cells with Magnetic Beads Loaded with Kras G12D (1-20) Neoantigen Library

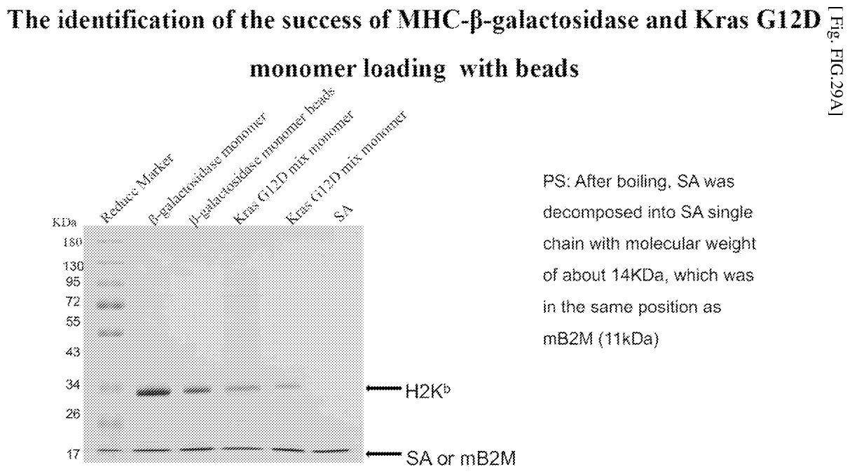

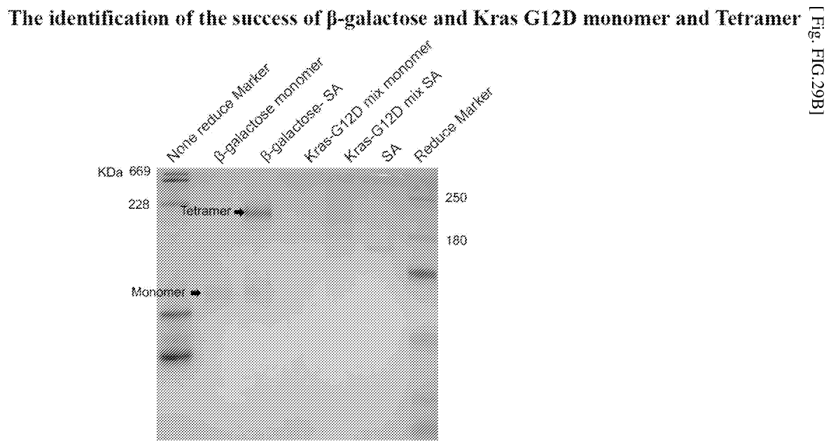

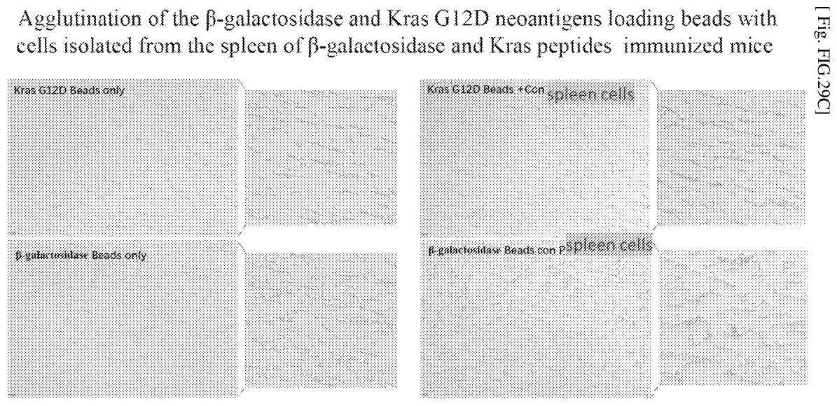

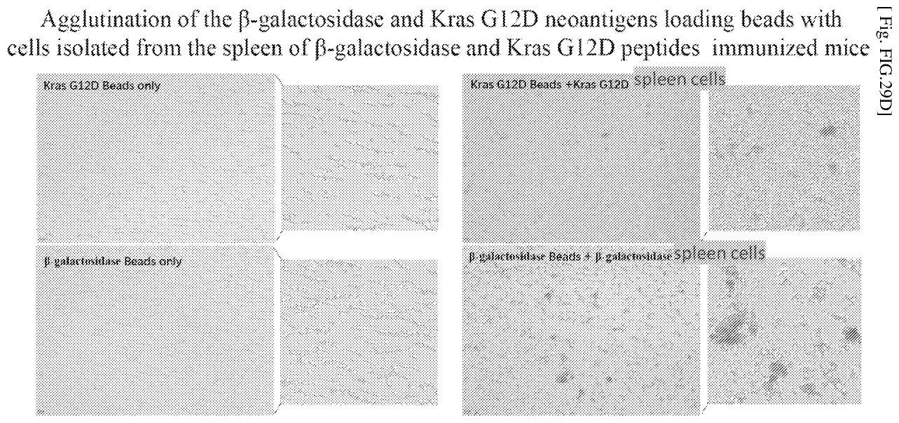

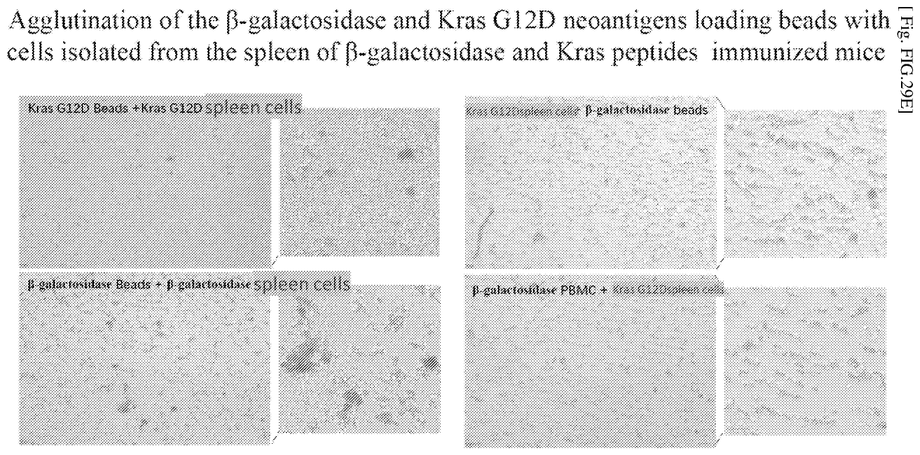

Similar methods discussed in Example 1A was applied for this example KRAS G12D (1-20) were used for immunization. As demonstrated in FIGS. 29A-29E, the cells from spleens from matched antigen peptide immunized mice were also able to specifically bind to beads associated with peptide specific MHC multimers.

### Example 7. Preparation of the MHC-Peptide Library Complexes

Materials: The following neoantigen libraries were synthesized by Nanjing Peptide Biotech Ltd. The Kras 20-peptide neoantigen library included Kras G12V (KV1-KV20) and Kras G12R (KR1). The Kras 50-peptide neoantigen library includes Kras G12V (KV1-KV20), Kras G12R (KR1-KR20), and Kras G12D (KD1-KV11). See FIG. 27C. The purity of both neoantigen libraries were above 95%. MHC heavy chain (HLA-A*02: 01) containing an His-tag and MHC light chain (hβ2m) were expressed and purified in Escherichia coli.

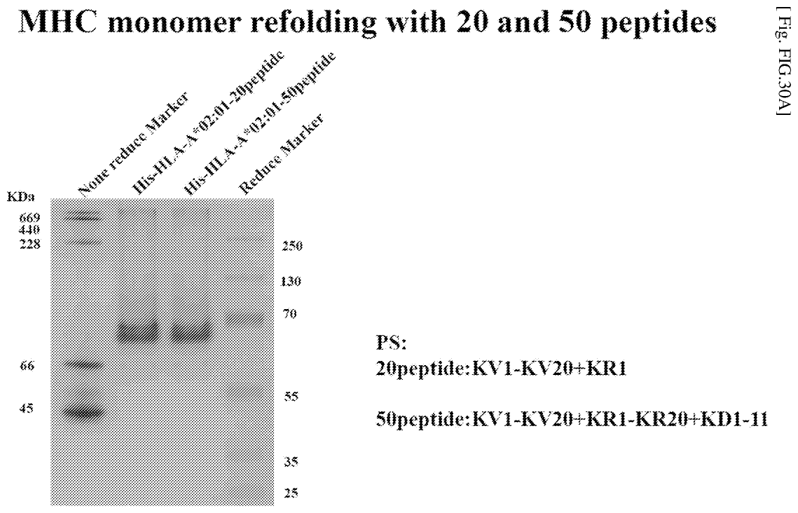

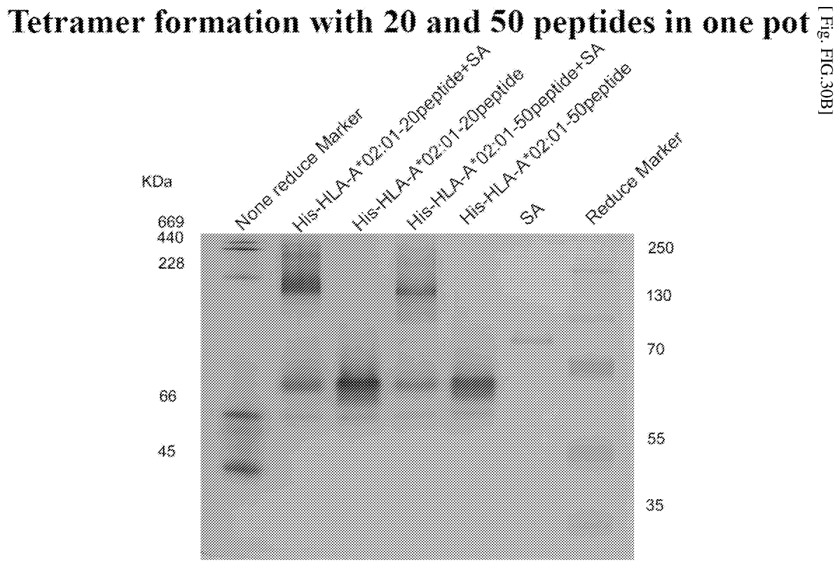

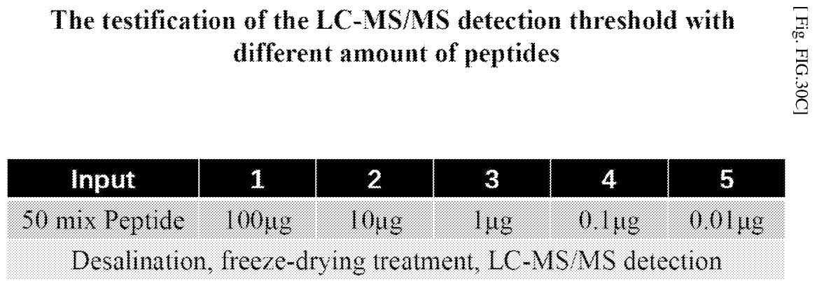

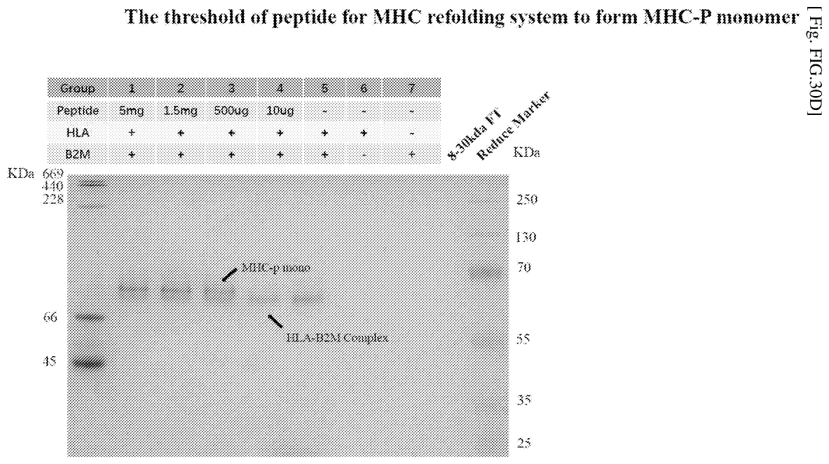

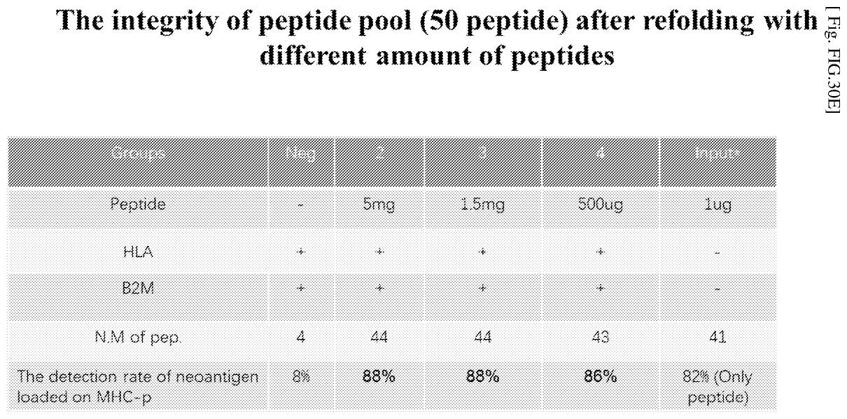

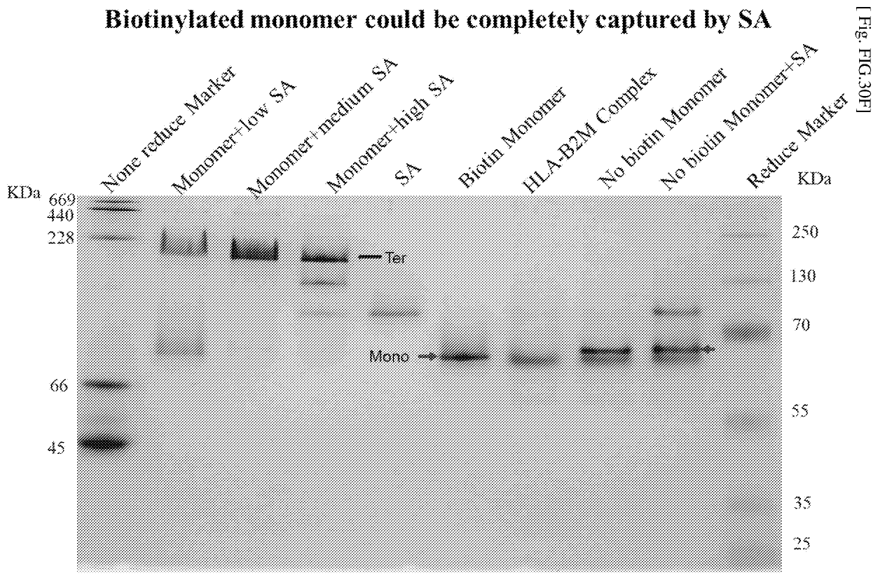

Formation of MHC-peptide monomer: In this experiment, the MHC-peptide monomer was formed following the preparation method described in Example 1, with the modification that HLA-A*02: 01 and human β2-microglobulin (hβ2m) were used as the MHC heavy and light chains, respectively. As shown in the non-reducing SDS-PAGE results in FIG. 30A, either a Kras 20-peptide library or a Kras 50-peptide library was mixed with the HLA-A*02: 01 and the hβ2m to form MHC-peptide monomer. Lane #2 showed the His-HLA-A*02: 01 in complex with the Kras 20-peptide library. Lane #3 showed the His-HLA-A*02: 01 in complex with the Kras 20-peptide library. Multiple bands were observed in the approximately 60-70 kDa range, confirming the presence of various peptides, which was attributed to the variation in molecular weight of the different peptides in the library.

Formation of MHC-peptide tetramer: In this experiment, the MHC-peptide monomer was formed following the preparation method described in Example 1, with the modification that HLA-A*02: 01 and human β2-microglobulin (hβ2m) were used as the MHC heavy and light chains, respectively. The biotinylation of the carried out following the suggested protocol in this reference (Altman, J. D., and Davis, M. M. 2016. MHC-peptide tetramers to visualize antigen-specific T cells. Curr. Protoc. Immunol. 115: 17.3.1-17.3.44). Subsequently, the MHC-peptide tetramer was formed by mixing in streptavidin. As shown in the non-reducing SDS-PAGE results in FIG. 30B, higher molecular weight bands above 70 kDa were observed in the presence of added streptavidin, as shown in Lane #2 and #4. In contrast, bands were only observed in the approximately 60-70 kDa range in the absence of added streptavidin. The MHC-peptide tetramers were formed regardless of either a Kras 20-peptide library or a Kras 50-peptide library was used.

Optimization of MHC to peptide ratio in the formation of MHC-peptide monomer and tetramer: A QC method was developed to identify bound peptides in the MHC-peptide complexes. To establish the detection limit for this QC method, a series dilution of the Kras 50-peptide library was prepared as shown in FIG. 30C, ranging in 100 μg, 10 μg, 1 μg, 0.1 μg, and 0.01 μg. The serially diluted peptides were then desalted and freeze-dried to remove interfering salts.

FIG. 30D showed the formation of the MHC-peptide monomer in the presence of a reducing amount of the Kras 50-peptide library of 50 peptides (specifically 5 mg, 1.5 mg, 500 ug, and 10 ug) in a 50 mL refolding system, corresponding to 100 mg/L, 30 mg/L, 10 mg/L, 0.2 mg/L conc. of peptides). As demonstrated in the SDS-PAGE, 10 μg of neoantigen was not sufficient to form the MHC-peptide monomer as indicated by the dominant presence of the lower band (e.g. HLA-A*02: 01 in complex with human β2-microglobulin), while 500 μg was adequate as indicated by the dominant presence of the higher band (e.g. HLA-A*02: 01 in complex with human β2-microglobulin and Kras 50-peptide library). Additionally, increasing the amount of neoantigens (such as to 5 mg or 1.5 mg) did not further improve the yield of the MHC-peptide monomer in the 50 mL refolding system as indicated by the unchanging ratio between the higher band and the lower band when the peptide was increased from 500 μg to 1.5 mg and 5.0 mg.

FIG. 30E showed a table for the detection rates of neoantigens loaded on the MHC-peptide monomer. In the control, where 1 μg of the Kras 50-peptide library was directly loaded onto the LC-MS/MS for analysis, 41 out of the 51 peptides in the library were identified, thus validating the LC-MS/MS method. In test samples 2, 3, and 4, where 5 mg, 1.5 mg, and 500 μg of the Kras 50-peptide library were allowed to form MHC-peptide complexes and then subjected to LC-MS/MS analysis, 44, 44, and 43 out of the 51 peptides in the Kras 50-peptide library were identified, respectively. As shown in the table, 500 μg is enough to form ≥85% MHC-p monomer in 50 mL refolding system.

After refolding under the described in Example 1 and SEC that purifies MHC-p complexes and removes excessive biotin, the detection rate of neoantigens that were loaded on MHC-p complexes was evaluated by LC-MS/MS. More than 70% of the neoantigen peptides were detected.

FIG. 30F further showed the formation of the MHC-peptide monomer and the MHC-peptide tetramer in the presence of streptavidin. Lane #5 showed the presence of the MHC-peptide monomer in the absence of streptavidin. Increasing the amount of MHC-peptide monomer to streptavidin ratio in the order of Lanes 4, 3, and 2 promoted the formation of MHC-peptide tetramers, as indicated by the presence of bands of progressively higher molecular weights. The presence of the MHC-peptide monomer in Lane 2 indicates an excess amount of MHC-peptide monomer in the binding mixture, and all four binding sites on the streptavidin have already been saturated with MHC-peptide monomer, thus preventing the formation of more. In contrast, without biotin attached to the MHC-peptide monomer, the MHC-peptide tetramer could not be formed with streptavidin, as shown in Lane #9. Lanes 6, 7, 8 were various controls, as biotinylated MHC-peptide monomer, HLA-A*02: 01 in complex with human β2-microglobulin MHC complex, and non-biotinylated MHC-peptide monomer respectively.

Collectively, FIGS. 30A-30F demonstrated that the MHC-peptide complexes, including both the MHC-peptide monomer and the MHC-peptide tetramer, can be formed from mixtures of a broader range peptides in one pot, therefore allowing scaling the assembly of heterogenous MHC-peptide molecules efficiently and effectively.

### Example 8. Preparation of the MHC-Peptide Library Complexes

Magnetic streptavidin beads (e.g., dynabeads of about 1 μm in diameter) were mixed with avi-tag-biotinylated MHC-p monomer with mixed peptides (e.g. heterogenous KRAS peptides targeting a specific mutation as discussed above) or heterogenous MHC molecules. See e.g., FIG. 27A. Because the streptavidin were embedded in beads, and three of the four avidin binding sites were available for binding with heterogenous MHC-p monomers.

Beads of different sizes (from about 100 nm to about 50 μm in diameter) have also been tested and shown working consistently in both ELISA and agglutination essay. Specifically, beads with a diameter of 100 nm, 2.8 μm, 8 μm, 20 μm, and 45 μm have been tested.

Histidine-nickel binding in lieu of streptavidin-biotin binding has also been tested for this method and demonstrated working.

Two ELISA tests were developed, and the difference was primarily based on (1) the type of MHC-peptide complex and (2) the binding site of HRP in the aggregates involving MHC-peptide complexes and immune cells. In the ELISA test protocol 1 (see FIG. 27A continued), the HRP was conjugated onto an Anti-CD8 antibody, which recognize the immune cells in the aggregates. The MHC-peptide monomer was used to load onto the beads. In the ELISA protocol 2 (see FIG. 27B), the HRP was conjugated onto the MHC-peptide multimer unit. The beads were used to load anti-CD8 antibody for pulling down the aggregates. Both ELISA test protocols and results were shown below.

Materials: The MHC-peptide monomer complexes with either the Kras G12V (KV1-KV16) neoantigen library or ovalbumin-derived peptide SIINFEKL were prepared following the protocol in Example 1. MHC heavy chain (H-2Kb) containing an Avi-tag and MHC light chain (β2m) were expressed and purified in Escherichia coli according to an published protocol (reference to 20004. xx application). HLA-A*02: 01/24: 02 was also expressed and purified in Escherichia coli. The beads loaded with the MHC-peptide monomer complexed was also prepared following the protocol in Example 6A.

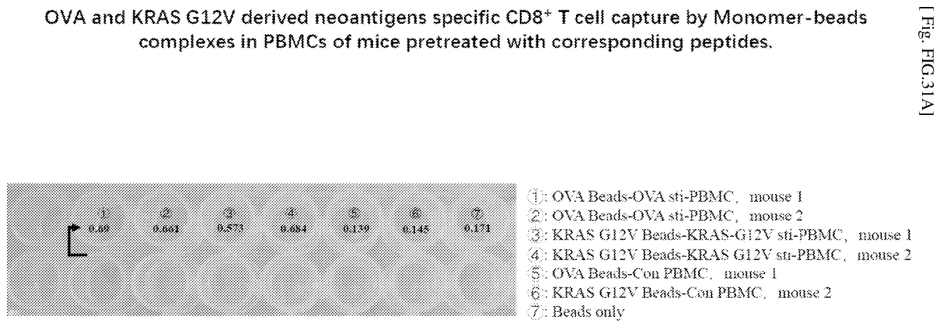

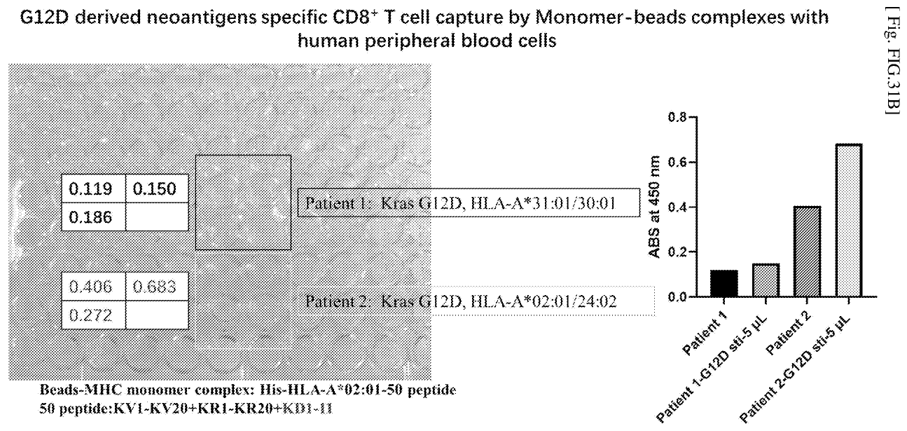

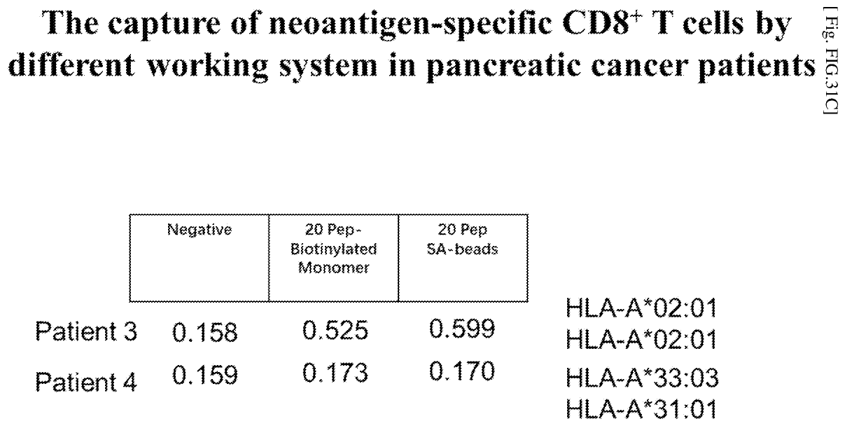

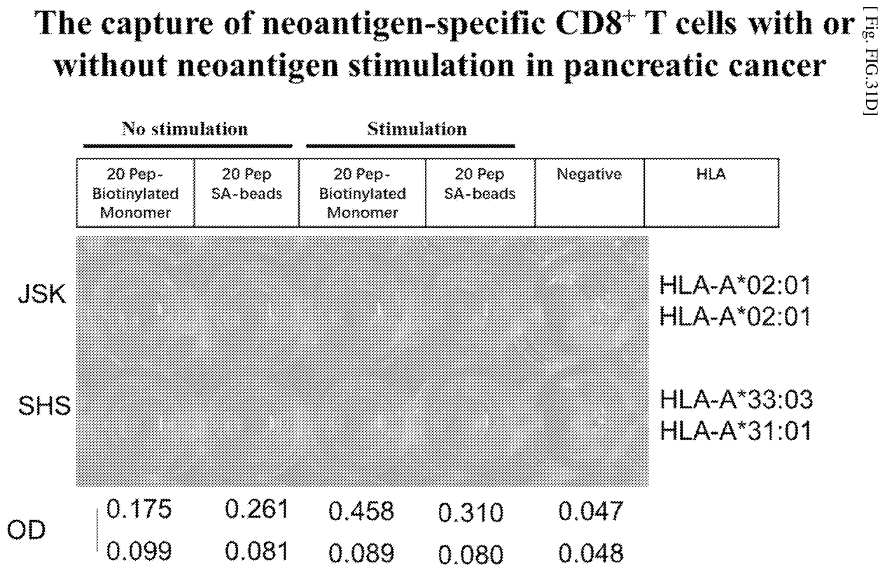

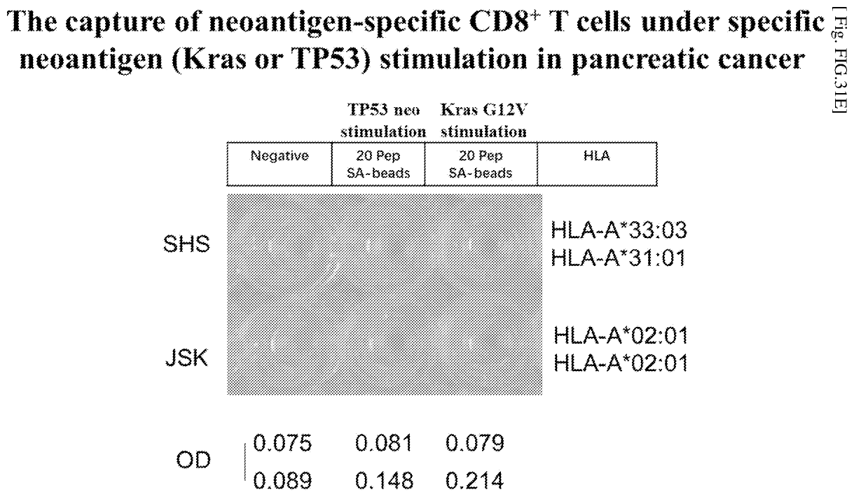

ELISA Test Protocol 1 and Results using Mouse PBMCs: As shown in FIG. 31A, 1×10⁵ cells were seed in 0.1 mL assay buffer in each well on 96 well plates. Then, H-2Kb/β2m coated magnetic beads, loaded with either the Kras G12V (KV1-KV16) neoantigen library or ovalbumin-derived peptide SIINFEKL, and anti-CD8 antibody conjugated with HRP were added to the cells. The 96 well plates were then allowed to incubate at room temperature for 0.5-1 hour. Then, the aggregates involving MHC-peptide complexes and PBMC cells aggregates were pulled down using a magnetic separator. After remove the unbound anti-CD8 antibody conjugated with HRP, the aggregates were washed thoroughly. Finally, the aggregates containing bound anti-CD8 antibody conjugated with HRP were developed using a TMB substrate. After the reaction was stopped, the 96 well plates were then measured at OD 450 nm. The quantification was then calculated based on the amount of anti-CD8 antibody conjugated with HRP bound to the immune cells. As shown in the control well #7 in FIG. 31A, in which only the beads were added, very little signal (OD450=0.171) was generated. When the beads, either loaded with the Kras G12V (KV1-KV16) neoantigen library or the Ovalbumin-derived peptide SIINFEKL, reacted with naïve PBMC cells, little signal (OD450=0.139 and 0.145) were generated in the control wells #6 and #5 in FIG. 31A respectively. However, when the beads, either loaded with the Kras G12V (KV1-KV16) neoantigen library or the Ovalbumin-derived peptide SIINFEKL, reacted with PBMC cells from C57BL/6J mice, immunized with matching peptides (either the Kras G12V (KV1-KV16) neoantigen library or the Ovalbumin-derived peptide SIINFEKL), strong signals (well #1, OD450-0.69, well #2, OD450-0.661; well #3, OD450-0.573; well #4, OD450=0.684) were observed. The results demonstrated that the PBMC cells present in the aggregates involving MHC-peptide complexes and immune cells were quantitatively measured.

ELISA Test Protocol 1 and Results using Human PBMCs: As shown in FIG. 31B, 1×10⁵ cells were seed in 0.1 mL assay buffer in each well on 96 well plates. Then, [[?]] μg HLA-A*02: 01/24: 02 coated magnetic beads, loaded with the Kras 50-peptide library, and anti-CD8 antibody conjugated with HRP were added to the cells. The 96 well plates were then allowed to incubate at room temperature for 0.5-1 hour. Then, the aggregates involving MHC-peptide complexes and PBMC cells aggregates were pulled down using a magnetic separator. After remove the unbound anti-CD8 antibody conjugated with HRP, the aggregates were washed thoroughly. Finally, the aggregates containing bound anti-CD8 antibody conjugated with HRP were developed using a TMB substrate. After the reaction was stopped, the 96 well plates were then measured at OD 450 nm. The quantification was then calculated based on the amount of anti-CD8 antibody conjugated with HRP bound to the immune cells. As shown in the first patient WRC sample in FIG. 31B, little signals (OD450<0.2) were generated because the first patient WRC has HLA-A*31: 01/30: 01, a different HLA type for coating the magnetic beads. In contrast, the second patient WRJ as shown in FIG. 31B, strong signals were observed because the second patient WRJ has the same HLA-A*02: 01/24: 02 for coating the magnetic beads. The results demonstrated that the human PBMC cells present in the aggregates involving MHC-peptide complexes and immune cells were also successfully measured.

The ELISA Test Protocol 1 demonstrated successful testing results for both Mouse and Human Peripheral Blood Mononuclear Cells (PBMCs). The ELISA protocol 1 also demonstrated the two separate targeting sites for modulating the interaction of MHC-peptide complexes and immune cells: at the MHC or at the peptide. In the test using the Mouse PBMCs, the modulation was achieved through the binding peptides (either the Kras G12V (KV1-KV16) neoantigen library or the Ovalbumin-derived peptide SIINFEKL). In the test using the Human PBMCs, the modulation was achieved through the MHC (either the HLA-A*31: 01/30: 01 or the HLA-A*02: 01/24: 02). Together, the two tests using the ELISA protocol 1 provided successful examples for using the MHC-peptide multimer units in testing the immune cells.

While ELISA Test Protocol 1 used MHC-peptide monomer complex in the test, ELISA Test Protocol 2 was further developed to use MHC-peptide tetramer complex in the test. Additionally, ELISA Test Protocol 2 simplified the testing protocol by conjugating the HRP directly onto the MHC-peptide tetramer complex, thereby reducing the total number of steps in the ELISA testing process.

As shown in FIG. 27B, ELISA Test Protocol 2 utilized an HRP conjugated MHC-peptide tetramer complex, followed by a simple step of mixing with immune cells. Then, the resulting MHC-peptide and immune cell aggregates could be pulled down magnetic beads conjugated with anti-CD3 antibody or anti-CD8 antibody. MHC-peptide tetramer complex was formed by reacting biotinylated MHC-peptide monomer with streptavidin, and the streptavidin could be unconjugated or could be conjugated onto beads (e.g., latex beads).

ELISA Test Protocol 2 and Results using Human PBMCs from Pancreatic Cancer Patients: Firstly, a number of HRP conjugated MHC-peptide complexes were prepared: (1) Condition 1 was a negative control, where no MHC-peptide complexes were added in the test; (2) Condition 2 was an HLA-A*02: 01 MHC bound with a 10-peptide library biotinylated monomer; (3) Condition 3 was an HLA-A*02: 01 MHC bound with a 50-peptide library biotinylated monomer; (4) Condition 5 was an HLA-A*02: 01 MHC bound with a 20-peptide library biotinylated monomer; (5) Condition 6 was an HLA-A*02: 01 MHC bound with a 20-peptide library biotinylated monomers bound on streptavidin beads; (6) Condition 7 was an HLA-A*02: 01 MHC bound with a 50-peptide library biotinylated monomers bound on Ni beads. 1.2×106 cells were seed in 0.1 mL assay buffer in each well on 96 well plates, followed by mixing in MHC-peptide complexes as prepared above. The 96 well plates were then allowed to incubate at room temperature for 0.5-1 hour. Then anti-CD8 antibody conjugated magnetic beads were used to pull down the aggregates involving MHC-peptide complexes and PBMC cells using a magnetic separator. After remove the unbound HRP conjugated MHC-peptide complexes, the aggregates were washed thoroughly. Finally, the aggregates containing bound HRP conjugated MHC-peptide complexes were developed using a TMB substrate. After the reaction was stopped, the 96 well plates were then measured at OD 450 nm. The quantification was then calculated based on the amount of HRP conjugated MHC-peptide complexes to the immune cells. As shown in the patient 1 in FIG. 31C, who had an HLA type of HLAA*02: 01. Strong signals (OD450 >0.5) were generated, indicating the PBMC cells from patent 1 were successfully identified. Interestingly, both the HLA-A*02: 01 MHC bound with a 20-peptide library streptavidin tetramer with and without on beads produced similar signal, further indicating the HLA-A*02: 01 MHC bound with a 20-peptide library streptavidin tetramer was responsible for recognizing the PBMC cells. In contrast, the patent 2 had a different HLA type of HLA-A*33: 01, therefore the PBMC cells from patent 2 were not able recognize HLA-A*02: 01 MHC bound with a 20-peptide library streptavidin tetramer and no signal (>0.2) was generated.

ELISA Test Protocol 2 and Results using Stimulated Human PBMCs from Pancreatic Cancer Patients: Firstly, a number of HRP conjugated MHC-peptide complexes were prepared: (1) Condition 5 was a negative control, where no MHC-peptide complexes were added in the test; (2) Condition 1 and 3 were an HLA-A*02: 01 MHC bound with a 20-peptide library streptavidin tetramer; (3) Condition 2 and 4 were an HLA-A*02: 01 MHC bound with a 20-peptide library streptavidin tetramer on beads. 1.2×10⁶ cells were seed in 0.1 mL assay buffer in each well on 96 well plates. In conditions 3-5, PBMC cells were stimulated before seeded onto the 96 well plates.

Method for stimulation: (1) Isolated 1×10⁶ PBMCs from blood of patients, resuspend PBMCs with 150 μL CTL-medium; (2) Add neoantigens pools at the working concentration of 0.1 ug/mL; (3) Add human IL-2 solution with working concentration of 50U/mL on day 2; (4) IL-2 was added for 50U/ml on day five; and (5) Detection on days 8-10.

After mixing in MHC-peptide complexes as prepared above, the 96 well plates were allowed to incubate at room temperature for 0.5-1 hour. Then anti-CD8 antibody conjugated magnetic beads were used to pull down the aggregates involving MHC-peptide complexes and PBMC cells using a magnetic separator. After remove the unbound HRP conjugated MHC-peptide complexes, the aggregates were washed thoroughly. Finally, the aggregates containing bound HRP conjugated MHC-peptide complexes were developed using a TMB substrate. After the reaction was stopped, the 96 well plates were then measured at OD 450 nm. The quantification was then calculated based on the amount of HRP conjugated MHC-peptide complexes to the immune cells. As shown in the patient JSK in FIGS. 31D-31E, who had an HLA type of HLAA*02: 01. Medium to strong signals (OD450 >0.2) were generated on all conditions, indicating the PBMC cells from patent JSK were successfully identified. Interestingly, both the HLA-A*02: 01 MHC bound with a 20-peptide library streptavidin tetramer with and without on beads produced similar signal, further indicating the HLA-A*02: 01 MHC bound with a 20-peptide library streptavidin tetramer was responsible for recognizing the PBMC cells. However, signals using stimulated PBMC cells nearly doubled compared to those un-stimulated PBMC cells, therefore proving the signal could be further boosted by stimulating the PBMC cells before the ELISA tests. In contrast, the patient SHS had a different HLA type of HLA-A*33: 01, therefore the PBMC cells from patent 2 were not able recognize HLA-A*02: 01 MHC bound with a 20-peptide library streptavidin tetramer and no signal (>0.2) was generated. Stimulating PBMC cells did not have any impact on the signal strength. However, when patient SHS' PBMC cells were stimulated with TP53 or Kras G12V neoantigens, the signal was significantly higher.

SEQUENCE TABLE

| SEQ ID | | |
|---|---|---|
| NO. | Description | Nucleotide or Amino Acid Sequence |
| 1. | Exemplary NSCLC | VYCEEYYLF |
| | neoantigenic peptide | |
| 2. | Exemplary NSCLC | YQANVVWKV |
| | neoantigenic peptide | |
| 3. | Exemplary NSCLC | ALYFNSQWK |
| | neoantigenic peptide | |
| 4. | Exemplary NSCLC | KLLSFHSV |
| | neoantigenic peptide | |
| 5. | Exemplary NSCLC | YLNEAVFNFV |
| | neoantigenic peptide | |
| 6. | Exemplary NSCLC | FYMHEYPEGW |
| | neoantigenic peptide | |
| 7. | Exemplary NSCLC | EIDLPRELEY |
| | neoantigenic peptide | |
| 8. | Exemplary NSCLC | KQDGYDSV |
| | neoantigenic peptide | |
| 9. | Exemplary NSCLC | LLQHYLLYR |
| | neoantigenic peptide | |
| 10. | Exemplary NSCLC | TTARMRTMR |
| | ncoantigenic peptide | |
| 11. | Exemplary NSCLC | HTFHLQDHH |
| | neoantigenic peptide | |
| 12. | Exemplary NSCLC | STTGATDLK |
| | neoantigenic peptide | |
| 13. | Exemplary NSCLC | MTFAETYPA |
| | neoantigenic peptide | |
| 14. | Exemplary NSCLC | NLLLIRGFK |
| | neoantigenic peptide | |
| 15. | Exemplary NSCLC | HGYFWFMGR |
| | neoantigonic peptide | |
| 16. | Exemplary NSCLC | FVLAALMEY |
| | neoantigenic peptide | |
| 17. | Exemplary NSCLC | RLRRLPVPR |
| | neoantigenic peptide | |
| 18. | Exemplary NSCLC | QSLVPAHPK |
| | neoantigenic peptide | |
| 19. | Exemplary NSCLC | RSFLSWDSR |
| | neoantigenic peptide | |
| 20. | Exemplary NSCLC | RIQGYIIEK |
| | neoantigenic peptide | |
| 21. | Exemplary NSCLC | ALNGMPLLK |
| | neoantigenic peptide | |
| 22. | Exemplary NSCLC | GTSESRETR |
| | neoantigenic peptide | |
| 23 | Exemplary NSCLC | IFSHPLYNI |
| | neoantigenic peptide | |
| 24. | Exemplary NSCLC | AAYLLFYQR |
| | neoantigenic peptide | |
| 25. | Exemplary NSCLC | SYVNILRAI |
| | neoantigenic peptide | |
| 26 | Exemplary NSCLC | RVEPVNYPK |
| | neoantigenic peptide | |
| 27. | Exemplary NSCLC | GYMGQQNEL |
| | neoantigenic peptide | |
| 28. | Exemplary NSCLC | RTQEARPPR |
| | neoantigonic peptide | |
| 29. | Exemplary NSCLC | RTASEDHPR |
| | neoantigenic peptide | |
| 30. | Exemplary NSCLC | VTIFVYDVK |
| | ncoantigenic peptide | |
| 31. | Exemplary NSCLC. | VVCIDAFLK |
| | neoantigenic peptide | |
| 32. | Exemplary NSCLC | STDQPVIPK |
| | neoantigenic peptide | |
| 33. | Exemplary NSCLC | SVAWPQDRR |
| | neoantigenic peptide | |
| 34. | Exemplary NSCLC | PTFGLSILK |
| | neoantigenic peptide | |
| 35. | Exemplary NSCLC | SYGLILLAF |
| | neoantigenic peptide | |
| 36. | Exemplary NSCLC | VEPLLFGTF |
| | neoantigenic peptide | |
| 37. | Exemplary NSCLC | YYLCLRHRL |
| | neoantigenic peptide | |
| 38. | Exemplary NSCLC | TSFPLDANK |
| | neoantigenic peptide | |
| 39. | Exemplary NSCLC | SLMVCNHDK |
| | neoantigenic peptide | |
| 40. | Exemplary NSCLC | SYIYILIII |
| | neoantigenic peptide | |
| 41. | Exemplary NSCLC | GVFRRCWEK |
| | neoantigenic peptide | |
| 42. | Exemplary NSCLC. | RSAAIASEK |
| | neoantigenic peptide | |
| 43. | Exemplary NSCLC | SSTHPHFVR |
| | neoantigenic peptide | |
| 44. | Exemplary NSCLC | NVLEINFIK |
| | neoantigenic peptide | |
| 45. | Exemplary NSCLC | SMVPVMYQK |
| | neoantigenic peptide | |
| 46. | Exemplary NSCLC | SVYCIGQRR |
| | neoantigenic peptide | |
| 47. | Exemplary NSCLC | YQMLSFVHK |
| | neoantigenie peptide | |
| 48. | Exemplary NSCLC | LYSFISRFI |
| | neoantigenic peptide | |
| 49. | Exemplary NSCLC | FSFRSCNFK |
| | neoantigenic peptide | |
| 50. | Exemplary NSCLC | ISYAKYFPK |
| | neoantigenic peptide | |
| 51 | Exemplary NSCLC | AEYQDMHSY |
| | neoantigenic peptide | |
| 52. | Exemplary NSCLC | VEHINISQDW |
| | neoantigenic peptide | |
| 53. | Exemplary NSCLC | RGRMQTASL |
| | neoantigenic peptide | |
| 54. | Exemplary NSCLC | VRINTARPV |
| | neoantigenic peptide | |
| 55. | Exemplary NSCLC | RRSMLFARH |
| | neoantigenic peptide | |
| 56. | Exemplary NSCLC | KTDTGVHATL |
| | neoantigenic peptide | |
| 57. | | KLVVVGAX |
| | | X: V, D, R, C, I, or A (Same for |
| | | SEQ ID Nos: 58-76) |
| 58. | | KLVVVGAXG |
| 59. | | KLVVVGAXGV |
| 60. | | KLVVVGAXGVG |
| 61. | | LVVVGAXG |
| 62. | | LVVVGAXGV |
| 63. | | LVVVGAXGVG |
| 64. | | LVVVGAXGVGK |
| 65. | | VVVGAXGV |
| 66. | | VVVGAXGVG |
| 67. | | VVVGAXGVGK |
| 68. | | VVVGAXGVGKS |
| 69. | | VVGAXGVG |
| 70. | | VVGAXGVGK |
| 71. | | VVGAXGVGKS |
| 72. | | VVGAXGVGKSA |
| 73. | | VGAXGVGK |
| 74. | | VGAXGVGKS |
| 75. | | VGAXGVGKSA |
| 76. | | VGAXGVGKSAL |
| 77. | | NLVPMVATV |
| 78. | | ELAGIGILTV |
| 79. | | RMFPNAPYL |
| 80. | | KLVVVGADGV |
| 81. | | LVVVGADGV |
| 82. | | VVGADGVGKSA |
| 83. | | VGADGVGKSAL |
| 84. | | VGADGVGKSA |
| 85. | | VVVGADGV |
| 86. | | VVVGADGVGK |
| 87. | | VVGADGVGK |
| 88. | | KLVVVGADG |
| 89. | | VVGADGVGKS |
| 90. | | KLVVVGAVG |
| 91. | | KLVVVGAVGV |
| 92. | | KLVVVGAVGVG |
| 93. | | LVVVGAVGV |
| 94. | | LVVVGAVGVGK |
| 95. | | VVVGAVGV |
| 96. | | VVVGAVGVG |
| 97. | | VVVGAVGVGK |
| 98. | | VVVGAVGVGKS |
| 99. | | VVGAVGVG |
| 100. | | VVGAVGVGK |
| 101. | | VVGAVGVGKS |
| 102. | | VVGAVGVGKSA |
| 103. | | KLVVVGAV |
| 104. | | LVVVGAVG |
| 105. | | VGAVGVGK |
| 106. | | VGAVGVGKS |
| 107. | | VGAVGVGKSA |

1. A method of analyzing a sample from one or more individuals for the presence or absence of immune cells capable of binding to an antigenic peptide, the method comprising:
a) immobilizing immune cells from the sample on a solid support, wherein the immune cells:
1) prior to immobilization, have been contacted with a bait composition comprising a display moiety comprising the antigenic peptide under a condition sufficient for an immune cell to bind to the display moiety; or
2) after the immobilization, are contacted with a bait composition comprising a display moiety comprising the antigenic peptide under a condition sufficient for an immune cell to bind to the display moiety;
b) contacting the immobilized immune cells with a detection agent recognizing the display moiety under a condition sufficient for the agent to bind to the display moiety; and
c) detecting the detection agent, thereby detecting the immune cells bound to the display moiety,
optionally wherein the method further comprises isolating immune cells bound to the display moiety or removing the display moiety or immune cells not bound to each other prior to step c).

2. A method of analyzing a sample from one or more individuals for the presence or absence of immune cells capable of binding to an antigenic peptide, the method comprising:
a) contacting the immune cells with a bait composition comprising a display moiety comprising the antigenic peptide under a condition sufficient for an immune cell to bind to the display moiety;
b) contacting the immune cells with a detection agent recognizing the display moiety under a condition sufficient for the agent to bind to the display moiety;
c) immobilizing the immune cells from the sample on a solid support, and
d) detecting the detection agent, thereby detecting the immune cells bound to the display moiety,
optionally wherein the method further comprises isolating immune cells bound to the display moiety or removing the display moiety or immune cells not bound to each other prior to step d).

3. The method of claim 1 or claim 2, wherein the sample is obtained from a single individual.

4. The method of claim 1 or claim 2, wherein the sample is a mixture a plurality of individual samples, each obtained from a different individual, optionally wherein prior to being combined to form the mixture each of the plurality of individual samples is separately processed such that the immune cells contained therein are associated with a unique sample barcode, optionally wherein method further comprises removing unbound molecule comprising the barcode, optionally wherein the method further comprises determining the unique barcode associated with the immune cells bound to the display moiety, thereby identifying the sample from which the immune cells are derived.

5. The method of any one of claims 1 and 3-4, wherein a) the immune cells have been contacted with a bait composition comprising a display moiety comprising an antigenic peptide under a condition sufficient for an immune cell to bind to the display moiety prior to the immobilization, b) the immune cells are contacted with a bait composition comprising a display moiety comprising an antigenic peptide under a condition sufficient for an immune cell to bind to the display moiety following the immobilization.

6. A method of analyzing a plurality of samples from one or more individuals for the presence or absence of immune cells capable of binding to an antigenic peptide, comprising:
a) separately processing each of the plurality of individual samples such that immune cells contained therein are associated with a unique sample barcode,
b) combining the plurality of processed samples to form a sample mixture,
c) contacting the sample mixture with a bait composition comprising a display moiety comprising an antigenic peptide under a condition sufficient for an immune cell to bind to the display moiety, thereby forming a bait-sample mixture,
d) detecting immune cells bound to the display moiety in the bait-sample mixture; and
e) determining the unique barcode associated with the immune cells bound to the display moiety, thereby identifying the sample from which the immune cells are derived,
optionally wherein the method further comprises isolating immune cells bound to the display moiety or removing the display moiety or immune cells not bound to each other prior to step d).

7. The method of any one of claims 1-6, wherein the immune cells comprise T cells, optionally the immune cells comprise no more than about 10%, 5%, 4%, 3%, 2% or 1% T cells specifically binding to antigenic peptide in the bait composition, optionally wherein the immune cells are selected from the group consisting of: a cytotoxic T cell, a memory T cell, and a tumor infiltrating T cell.

8. The method of any one of claims 1-7, wherein the method further comprises removing unbound display moiety following contacting the immune cells with the agent recognizing the display moiety and prior to detecting the immune cells bound to the display moiety.

9. The method of any one of claims 1-8, wherein the antigenic peptide is associated with a cancer or tumor antigen, optionally wherein the antigenic peptide is a neoantigen peptide, optionally wherein the sample is obtained from one or more individuals exhibiting no pathological symptom of a cancer.

10. The method of any one of claims 1-5 and 7-9, wherein the immune cells are immobilized on the solid substrate such as a bead, optionally wherein the bead is associated with an antibody recognizing the immune cells, optionally wherein the antibody is a CD8 antibody.

11. The method of any one of claims 1-10, wherein the display moiety comprises two or more antigenic peptides, optionally wherein the display moiety comprises four antigenic peptides, optionally wherein the two or more antigenic peptides in the display moiety are the same.

12. The method of claims 9-11, wherein the display moiety comprises a library of antigenic peptides for a mutation associated with a cancer or tumor, wherein the library comprises at least about 5, 10, 12, 15, 18 or 20 distinct neoantigenic peptides.

13. The method of any one of claims 1-12, wherein the display moiety comprises an MHC molecule complexed with the antigenic peptide (i.e., MHC-peptide complex), wherein the MHC molecule comprises a MHC class I molecule, optionally wherein the MHC molecule comprises a MHC multimer, further optionally the MHC multimer is a MHC dimer, a MHC trimer, a MHC tetramer, or a MHC dextramer.

14. The method of claim 13, wherein the display moiety comprises two or more different kinds of MHC class I molecules selected from the group consisting of
a) HLA-A*24: 02, HLA-A*11: 01, HLA-A*02: 01, and HLA-A*03: 01, or
b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03.

14. The method of any one of claims 1-13, wherein the display moiety comprises a particle, optionally wherein the particle in the display moiety is selected from the group consisting of: a surface, a nanoparticle, a bead, and a polymer.

15. The method of claim 14, wherein the particle comprises a bead, wherein the bead has a diameter of at least about 0.1 μm, 0.25 μm, 0.5 μm or 1 μm.

16. The method of any one of claims 14-15, wherein the antigenic peptide or MHC a) is directly attached to the particle in the display moiety or b) is attached to the particle in the display moiety via a binding pair comprising a first binding component attached to the antigenic peptide or MHC and a second binding component bound to the particle.

17. The method of any one of claims 1-16, wherein the method further comprises a) isolating immune cells bound to the display moiety or removing the display moiety or immune cells not bound to each other, and/or b) phenotyping immune cells bound to the display moiety.

18. The method of any one of claims 1-17, wherein the individual is a human.

19. The method of any one of claims 9-18, wherein the individual
a) has not previously been diagnosed as having a cancer, optionally wherein the individual is at risk of having cancer, or
b) the individual has been previously treated for cancer and exhibits no pathological symptom of a cancer after the treatment.

20. The method of any one of claims 1-19, wherein the sample is selected from the group consisting of: blood, plasma, and a peripheral blood mononuclear cell (PBMC) sample.

21. The method of any one of claims 1-20, wherein the bait composition comprises a plurality of different display moieties, optionally wherein the plurality of different display moieties in the bait composition comprises at least two different display moieties, each comprising a different MHC molecule.

22. A method of detecting antigen-specific immune cells in an individual, comprising: analyzing a sample from the individual according to any one of claims 1-21,
wherein a predetermined characteristic of the immune cells is indicative of a disease or a disorder in the individual.

23. The method of claim 22, wherein the predetermined characteristic of the immune cell comprises the presence of the immune cells, optionally wherein a) the presence of immune cells specifically binds to a bait composition comprising one or more neoantigens is indicative of the presence of a cancer, b) the presence of immune cells specifically binds to a bait composition comprising one or more antigens of a pathogen (e.g., virus, bacteria, fungus) is indicative of the presence of an infection of the pathogen, or c) the presence of immune cells specifically binds to a bait composition comprising one or more autoantigens associated with an autoimmune disease or disorder is indicative of the presence of the immune disease or disorder.

24. A method of detecting residual cancer in an individual, wherein the individual has been previously treated with an anti-cancer therapy and exhibits no pathological symptom of cancer after treatment, the method comprising analyzing a post-treatment sample from the individual according to the method of any one of claims 9-21,
wherein a predetermined characteristic of the immune cell from the post-treatment sample is indicative of residual cancer in the individual.

25. A method of treating a cancer in an individual, comprising
a) diagnosing the individual as having cancer according to the method of claim 23; and
b) subjecting the individual to an anti-cancer therapy.

26. A method of treating an infection in an individual, comprising
a) diagnosing the individual as having an infection by a pathogen according to the method of claim 23; and
b) subjecting the individual to a therapy for the pathogen.

27. A method of treating an autoimmune disease or disorder in an individual, comprising
a) diagnosing the individual as having an autoimmune disease or disorder according to the method of claim 23; and
b) subjecting the individual to a therapy for the disease or disorder.

28. A display moiety comprising a particle associated with a plurality of MHC-peptide complexes, wherein the plurality of MHC-peptide complexes comprises no less than or more than 2, 3, 4, 5, 6, 8, 9, 10, or 12 MHC-peptide complexes, wherein each of the MHC-peptide complex comprises at least one MHC molecule complexed with a peptide, wherein at least two or more of the MHC-peptide complexes are different.

29. The display moiety of claim 28, wherein one or more of the plurality of MHC-peptide complexes is an MHC-peptide monomer, an MHC-peptide dimer, an MHC-peptide trimer, an MHC-peptide tetramer, an MHC-peptide pentamer, an MHC-peptide octamer, an MHC-peptide dextramer, or an MHC-peptide dodecamer.

30. The display moiety of claim 28 or claim 29, wherein at least one MHC-peptide complex in the plurality of MHC-peptide complexes has two distinct MHC-peptide monomers, optionally the at least one MHC complex is an MHC-peptide dimer, an MHC-peptide trimer, or an MHC-peptide tetramer.

31. The display moiety of any one of claims 28-30, wherein the plurality of MHC-peptide complexes comprises at least 20 MHC-peptide complexes, optionally wherein the plurality of MHC-peptide complexes comprises at least 50 or 100 MHC-peptide complexes.

32. The display moiety of any one of claims 28-31, wherein the MHC-peptide complex is attached to the particle via a binding pair comprising a first binding component attached to the MHC-peptide complex or a unit thereof and a second binding component bound to the particle, optionally wherein the first binding component comprises a biotin, and the second binding component comprises streptavidin.

33. The display moiety of any one of claims 28-32, wherein the particle
a) is solid;
b) has a diameter of about 0.1 μm to about 50 μm (e.g., at least 0.1 μm, 0.2 μm, 0.25 μm, 0.5 μm, 0.75 μm, or 1 μm, optionally wherein the particle has a diameter of about 0.5 μm to about 20 μm, optionally wherein the particle has a diameter of about 0.5 μm to about 10 μm, further optionally wherein the particle has a diameter of about 1 μm;
c) has a surface area of about 1 μm² to about 20 μm²;
d) has a number of about 10 to about 10000 second binding component associated with it; e) the particle is spherical, optionally wherein the particle is a bead; and/or
f) is magnetic.

34. The display moiety of any one of claims 28-33, wherein display moiety comprises at least two different MHC molecules, optionally wherein
a) the at least two different MHC molecules comprises two different MHC class I molecules;
b) the at least two different MHC molecules comprises two different MHC class II molecules; and/or
c) the at least two different MHC molecules comprises both an MHC class I molecule and an MHC class II molecule.

35. The display moiety of any one of claims 28-34, wherein:
a) the display moiety comprises an HLA-A molecule, further optionally wherein the HLA-A molecule comprises a mutation that reduces its binding to CD8, further optionally where the HLA-A molecule comprises an HLA A2 heavy chain with a A245V mutation;
b) the display moiety comprises at least two MHC class I molecules selected from the group consisting of a) HLA-A*24: 02, HLA-A*11: 01, HLA-A*02: 01, and HLA-A*03: 01, or b) HLA-A*69: 01, HLA-A*31: 01, HLA-A*29: 01HLA-A*33: 02, HLA-A*02: 06, HLA-A*02: 07, HLA-A*30: 01, HLA-A*01: 01, HLA-A*02: 03, HLA-A*33: 03; and/or
c) the display moiety comprises an MHC class II molecule selected from the group consisting of HLA-DQ and HLA-DR.

36. The display moiety of any one of claims 28-35, wherein:
a) the peptide is associated with a cancer or tumor antigen, optionally wherein the peptide is associated with a neoantigen;
b) the peptide is associated with an antigen of a pathogen, optionally wherein the pathogen is a virus, bacteria of fungus;
c) the peptide is associated with an autoantigen;
d) the display moiety comprises at least two distinct peptides;
optionally wherein the display moiety comprises at least five distinct peptides, optionally wherein the display moiety comprises at least 2, 5, or 10 distinct peptides; and/or
e) the display moiety does not comprise a fluorochrome or fluorophore.

37. A bait composition comprising the display moiety of any one of claims 28-36.

38. The bait composition of claim 37, wherein:
a) the bait composition comprises at least two different display moieties;
b) the bait composition comprises an HLA-A molecule, an HLA-B molecule, and an HLA-C molecule;
c) the bait composition comprises HLA-A*24: 02, HLA-A*11: 01, HLA-A*02: 01, and HLA-A*03: 01;
d) the bait composition comprises an HLA-DP molecule, an HLA-DQ molecule, and an HLA-DR molecule;
e) the bait composition comprises at least 25, 50, 100, 150, 200, or 250 different peptides;
f) the peptides in the bait composition are associated with at least 25, 50, 75, 100, 125, 150, 175, 200, 225, or 250 different antigens;
g) the bait composition comprises at least 2, 3, 4, 5, 6, or 7 different overlapping peptides associated a mutation in a gene, optionally wherein the gene is an oncogene;
h) the peptides in the bait composition are associated with mutations in at least two, three, four, five or six genes, optionally wherein the genes are oncogenes; and/or
i) the bait composition does not comprise a fluorochrome or fluorophore.

39. A method of analyzing immune cells in a sample from an individual, comprising:
a) contacting a plurality of immune cells with the display moiety of any one of claims 28-36 or a bait composition of claim 37 or claim 38,
b) detecting or analyzing the one or more immune cells that bind to one or more of the display moieties.

40. The method of claim 39, wherein:
a) the immune cells comprise T cells;
b) the immune cells are selected from the group consisting of: cytotoxic T cells, memory T cells, and tumor infiltrating T cells, optionally wherein the T cells are CD4+ T cells and/or CD8+ T cells;
c) the immune cells comprise B cells; and/or
d) the immune cells comprise a mixture of immune cells comprising T cells, B cells, macrophage cells, or dendritic cells, or combinations thereof.

41. The method of claim 39 or claim 40, wherein:
a) the sample is selected from the group consisting of: blood, plasma, and a peripheral blood mononuclear cell (PMBC) sample; and/or
b) the sample is obtained from a diseased tissue, optionally wherein the diseased tissue is a cancer tissue or tumor tissue.

42. The method of any one of claims 39-41, wherein a) the sample is obtained from a single individual, or b) the sample is a mixture a plurality of individual samples, each obtained from a different individual.

43. The method of any one of claims 39-42, wherein the individual a) is a human, and/or b) has no indication of a disease, optionally wherein the disease is a cancer.

44. The method of any one of claims 39-43, wherein detecting or analyzing the immune cells that bind to one or more display moieties comprises observing the agglutination of immune cells and the one or more display moieties.

45. The method of any one of claims 39-44, wherein detecting or analyzing the immune cells that bind to one or more display moieties comprises 1) immobilizing immune cells on a solid substrate prior to or after step a), 2) contacting the immobilized immune cells with a detection agent recognizing the display moieties under a condition sufficient for the agent to bind to the display moieties; and 3) detecting the detection agent, thereby detecting the immune cells bound to the display moieties.


SEQUENCE LISTING (107 sequences)

SEQ ID NO: 1 - protein, 9 aa, synthetic construct
VYCEEYYLF

SEQ ID NO: 2 - protein, 9 aa, synthetic construct
YQANVVWKV

SEQ ID NO: 3 - protein, 9 aa, synthetic construct
ALYFNSQWK

SEQ ID NO: 4 - protein, 8 aa, synthetic construct
KLLSFHSV

SEQ ID NO: 5 - protein, 10 aa, synthetic construct
YLNEAVFNFV

SEQ ID NO: 6 - protein, 10 aa, synthetic construct
FYMHEYPEGW

SEQ ID NO: 7 - protein, 10 aa, synthetic construct
EIDLPRELEY

SEQ ID NO: 8 - protein, 8 aa, synthetic construct
KQDGYDSV

SEQ ID NO: 9 - protein, 9 aa, synthetic construct
LLQHYLLYR

SEQ ID NO: 10 - protein, 9 aa, synthetic construct
TTARMRTMR

SEQ ID NO: 11 - protein, 9 aa, synthetic construct
HTFHLQDHH

SEQ ID NO: 12 - protein, 9 aa, synthetic construct
STTGATDLK

SEQ ID NO: 13 - protein, 9 aa, synthetic construct
MTFAETYPA

SEQ ID NO: 14 - protein, 9 aa, synthetic construct
NLLLIRGFK

SEQ ID NO: 15 - protein, 9 aa, synthetic construct
HGYFWFMGR

SEQ ID NO: 16 - protein, 9 aa, synthetic construct
FVLAALMEY

SEQ ID NO: 17 - protein, 9 aa, synthetic construct
RLRRLPVPR

SEQ ID NO: 18 - protein, 9 aa, synthetic construct
QSLVPAHPK

SEQ ID NO: 19 - protein, 9 aa, synthetic construct
RSFLSWDSR

SEQ ID NO: 20 - protein, 9 aa, synthetic construct
RIQGYIIEK

SEQ ID NO: 21 - protein, 9 aa, synthetic construct
ALNGMPLLK

SEQ ID NO: 22 - protein, 9 aa, synthetic construct
GTSESRETR

SEQ ID NO: 23 - protein, 9 aa, synthetic construct
IFSHPLYNI

SEQ ID NO: 24 - protein, 9 aa, synthetic construct
AAYLLFYQR

SEQ ID NO: 25 - protein, 9 aa, synthetic construct
SYVNILRAI

SEQ ID NO: 26 - protein, 9 aa, synthetic construct
RVEPVNYPK

SEQ ID NO: 27 - protein, 9 aa, synthetic construct
GYMGQQNEL

SEQ ID NO: 28 - protein, 9 aa, synthetic construct
RTQEARPPR

SEQ ID NO: 29 - protein, 9 aa, synthetic construct
RTASEDHPR

SEQ ID NO: 30 - protein, 9 aa, synthetic construct
VTIFVYDVK

SEQ ID NO: 31 - protein, 9 aa, synthetic construct
VVCIDAFLK

SEQ ID NO: 32 - protein, 9 aa, synthetic construct
STDQPVIPK

SEQ ID NO: 33 - protein, 9 aa, synthetic construct
SVAWPQDRR

SEQ ID NO: 34 - protein, 9 aa, synthetic construct
PTFGLSILK

SEQ ID NO: 35 - protein, 9 aa, synthetic construct
SYGLILLAF

SEQ ID NO: 36 - protein, 9 aa, synthetic construct
VFPLLFGTF

SEQ ID NO: 37 - protein, 9 aa, synthetic construct
YYLCLRHRL

SEQ ID NO: 38 - protein, 9 aa, synthetic construct
TSFPLDANK

SEQ ID NO: 39 - protein, 9 aa, synthetic construct
SLMVCNHDK

SEQ ID NO: 40 - protein, 9 aa, synthetic construct
SYIYILIII

SEQ ID NO: 41 - protein, 9 aa, synthetic construct
GVFRRCWEK

SEQ ID NO: 42 - protein, 9 aa, synthetic construct
RSAAIASEK

SEQ ID NO: 43 - protein, 9 aa, synthetic construct
SSTHPHFVR

SEQ ID NO: 44 - protein, 9 aa, synthetic construct
NVLEINFIK

SEQ ID NO: 45 - protein, 9 aa, synthetic construct
SMVPVMYQK

SEQ ID NO: 46 - protein, 9 aa, synthetic construct
SVYCIGQRR

SEQ ID NO: 47 - protein, 9 aa, synthetic construct
YQMLSFVHK

SEQ ID NO: 48 - protein, 9 aa, synthetic construct
LYSFISRFI

SEQ ID NO: 49 - protein, 9 aa, synthetic construct
FSFRSCNFK

SEQ ID NO: 50 - protein, 9 aa, synthetic construct
ISYAKYFPK

SEQ ID NO: 51 - protein, 9 aa, synthetic construct
AEYQDMHSY

SEQ ID NO: 52 - protein, 10 aa, synthetic construct
VEHINISQDW

SEQ ID NO: 53 - protein, 9 aa, synthetic construct
RGRMQTASL

SEQ ID NO: 54 - protein, 9 aa, synthetic construct
VRINTARPV

SEQ ID NO: 55 - protein, 9 aa, synthetic construct
RRSMLFARH

SEQ ID NO: 56 - protein, 10 aa, synthetic construct
KTDTGVHATL

SEQ ID NO: 57 - protein, 8 aa, synthetic construct
  VARIANT 8: note = V, D, R, C, I, or A
KLVVVGAX

SEQ ID NO: 58 - protein, 9 aa, synthetic construct
  VARIANT 8: note = V, D, R, C, I, or A
KLVVVGAXG

SEQ ID NO: 59 - protein, 10 aa, synthetic construct
  VARIANT 8: note = V, D, R, C, I, or A
KLVVVGAXGV

SEQ ID NO: 60 - protein, 11 aa, synthetic construct
  VARIANT 8: note = V, D, R, C, I, or A
KLVVVGAXGVG

SEQ ID NO: 61 - protein, 8 aa, synthetic construct
  VARIANT 7: note = V, D, R, C, I, or A
LVVVGAXG

SEQ ID NO: 62 - protein, 9 aa, synthetic construct
  VARIANT 7: note = V, D, R, C, I, or A
LVVVGAXGV

SEQ ID NO: 63 - protein, 10 aa, synthetic construct
  VARIANT 7: note = V, D, R, C, I, or A
LVVVGAXGVG

SEQ ID NO: 64 - protein, 11 aa, synthetic construct
  VARIANT 7: note = V, D, R, C, I, or A
LVVVGAXGVGK

SEQ ID NO: 65 - protein, 8 aa, synthetic construct
  VARIANT 6: note = V, D, R, C, I, or A
VVVGAXGV

SEQ ID NO: 66 - protein, 9 aa, synthetic construct
  VARIANT 6: note = V, D, R, C, I, or A
VVVGAXGVG

SEQ ID NO: 67 - protein, 10 aa, synthetic construct
  VARIANT 6: note = V, D, R, C, I, or A
VVVGAXGVGK

SEQ ID NO: 68 - protein, 11 aa, synthetic construct
  VARIANT 6: note = V, D, R, C, I, or A
VVVGAXGVGKS

SEQ ID NO: 69 - protein, 8 aa, synthetic construct
  VARIANT 5: note = V, D, R, C, I, or A
VVGAXGVG

SEQ ID NO: 70 - protein, 9 aa, synthetic construct
  VARIANT 5: note = V, D, R, C, I, or A
VVGAXGVGK

SEQ ID NO: 71 - protein, 10 aa, synthetic construct
  VARIANT 5: note = V, D, R, C, I, or A
VVGAXGVGKS

SEQ ID NO: 72 - protein, 11 aa, synthetic construct
  VARIANT 5: note = V, D, R, C, I, or A
VVGAXGVGKSA

SEQ ID NO: 73 - protein, 8 aa, synthetic construct
  VARIANT 4: note = V, D, R, C, I, or A
VGAXGVGK

SEQ ID NO: 74 - protein, 9 aa, synthetic construct
  VARIANT 4: note = V, D, R, C, I, or A
VGAXGVGKS

SEQ ID NO: 75 - protein, 10 aa, synthetic construct
  VARIANT 4: note = V, D, R, C, I, or A
VGAXGVGKSA

SEQ ID NO: 76 - protein, 11 aa, synthetic construct
  VARIANT 4: note = V, D, R, C, I, or A
VGAXGVGKSAL

SEQ ID NO: 77 - protein, 9 aa, synthetic construct
NLVPMVATV

SEQ ID NO: 78 - protein, 10 aa, synthetic construct
ELAGIGILTV

SEQ ID NO: 79 - protein, 9 aa, synthetic construct
RMFPNAPYL

SEQ ID NO: 80 - protein, 10 aa, synthetic construct
KLVVVGADGV

SEQ ID NO: 81 - protein, 9 aa, synthetic construct
LVVVGADGV

SEQ ID NO: 82 - protein, 11 aa, synthetic construct
VVGADGVGKSA

SEQ ID NO: 83 - protein, 11 aa, synthetic construct
VGADGVGKSAL

SEQ ID NO: 84 - protein, 10 aa, synthetic construct
VGADGVGKSA

SEQ ID NO: 85 - protein, 8 aa, synthetic construct
VVVGADGV

SEQ ID NO: 86 - protein, 10 aa, synthetic construct
VVVGADGVGK

SEQ ID NO: 87 - protein, 9 aa, synthetic construct
VVGADGVGK

SEQ ID NO: 88 - protein, 9 aa, synthetic construct
KLVVVGADG

SEQ ID NO: 89 - protein, 10 aa, synthetic construct
VVGADGVGKS

SEQ ID NO: 90 - protein, 9 aa, synthetic construct
KLVVVGAVG

SEQ ID NO: 91 - protein, 10 aa, synthetic construct
KLVVVGAVGV

SEQ ID NO: 92 - protein, 11 aa, synthetic construct
KLVVVGAVGVG

SEQ ID NO: 93 - protein, 9 aa, synthetic construct
LVVVGAVGV

SEQ ID NO: 94 - protein, 11 aa, synthetic construct
LVVVGAVGVGK

SEQ ID NO: 95 - protein, 8 aa, synthetic construct
VVVGAVGV

SEQ ID NO: 96 - protein, 9 aa, synthetic construct
VVVGAVGVG

SEQ ID NO: 97 - protein, 10 aa, synthetic construct
VVVGAVGVGK

SEQ ID NO: 98 - protein, 11 aa, synthetic construct
VVVGAVGVGKS

SEQ ID NO: 99 - protein, 8 aa, synthetic construct
VVGAVGVG

SEQ ID NO: 100 - protein, 9 aa, synthetic construct
VVGAVGVGK

SEQ ID NO: 101 - protein, 10 aa, synthetic construct
VVGAVGVGKS

SEQ ID NO: 102 - protein, 11 aa, synthetic construct
VVGAVGVGKSA

SEQ ID NO: 103 - protein, 8 aa, synthetic construct
KLVVVGAV

SEQ ID NO: 104 - protein, 8 aa, synthetic construct
LVVVGAVG

SEQ ID NO: 105 - protein, 8 aa, synthetic construct
VGAVGVGK

SEQ ID NO: 106 - protein, 9 aa, synthetic construct
VGAVGVGKS

SEQ ID NO: 107 - protein, 10 aa, synthetic construct
VGAVGVGKSA